(19) United States
(12) Patent Application Publication
(10) Pub. No.: US 2015/0064172 A1
(43) Pub. Date: Mar. 5, 2015

(54) METHODS OF TREATING A DISEASE OR DISORDER ASSOCIATED WITH BRUTON'S TYROSINE KINASE

(71) Applicant: Celgene Avilomics Research, Inc., Bedford, MA (US)

(72) Inventors: Tom Daniel, LaJolla, CA (US); Kenichi Takeshita, New York, NY (US); Kenneth Foon, Summit, NJ (US); Jay Mei, North Wales, PA (US)

(73) Assignee: Celgene Avilomics Research, Inc., Bedford, MA (US)

(21) Appl. No.: 14/084,174

(22) Filed: Nov. 19, 2013

Related U.S. Application Data
(60) Provisional application No. 61/870,718, filed on Aug. 27, 2013.
(60) Provisional application No. 61/870,720, filed on Aug. 27, 2013.

(51) Int. Cl. A61K 31/505; A61K 31/454; A61K 39/395

(52) U.S. Cl. CPC A61K 31/505; A61K 39/3955; A61K 31/454
USPC 424/133.1; 514/275

(57) ABSTRACT

The present invention provides methods of treating, stabilizing or lessening the severity or progression of a disease or disorder associated with BTK.

### CROSS REFERENCE TO RELATED APPLICATIONS

The present application claims priority to U.S. provisional application Nos. 61/870,718, filed Aug. 27, 2013, and 61/870,720, filed Aug. 27, 2013, the entirety of each of which is hereby incorporated by reference.

### FIELD OF THE INVENTION

The present invention provides methods of treating, stabilizing or lessening the severity or progression of a disease or disorder associated with Bruton's Tyrosine Kinase (“BTK”).

### BACKGROUND OF THE INVENTION

The search for new therapeutic agents has been greatly aided in recent years by a better understanding of the structure of enzymes and other biomolecules associated with diseases. One important class of enzymes that has been the subject of extensive study is protein kinases.

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxin, and H₂O₂), cytokines (e.g., interleukin-1 (IL-1) and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, glucose metabolism, control of protein synthesis, and regulation of the cell cycle.

Many diseases are associated with abnormal cellular responses triggered by protein kinase-mediated events as described above. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases. Accordingly, there remains a need to find protein kinase inhibitors useful as therapeutic agents.

### SUMMARY OF THE INVENTION

Chronic lymphocytic leukemia (CLL) is a lymphoproliferative malignancy characterized by progressive accumulation of morphologically mature but functionally incompetent lymphocytes in the blood, bone marrow, and lymphoid tissues. It affects mainly elderly individuals with the median age at presentation of 65 to 70 years. Small lymphocytic lymphoma (SLL) and CLL are generally considered a different manifestation of the same disease. While CLL is found in the blood and bone marrow, SLL presents primarily in the lymph nodes. The clinical course of CLL/SLL ranges from indolent disease with long-term survival over 12 years to aggressive disease with median survival of 2 years. The average age of diagnosis with CLL/SLL is approximately 60 years.

Despite newly approved therapeutic agents and combination therapies, CLL/SLL remains an incurable disease and most patients eventually relapse and/or die. Improved and novel combination treatments for subjects with CLL/SLL requiring treatment remain an unmet medical need.

Bruton's tyrosine kinase (Btk) is a non-receptor tyrosine kinase with restricted cellular expression largely limited to B-lymphocytes, monocytes, and mast cells or basophils. Btk is a critical component of the B-cell receptor (BCR) signaling network and is crucial for B-cell development. Investigation has revealed that some B-cell malignancies, including diseases such as CLL/SLL, depend on BCR signaling, suggesting that interruption of such signaling could be a promising therapeutic opportunity. Recently, clinical anti-tumor responses in various B-cell non-Hodgkin's Lymphoma (NHL) and CLL/SLL have been reported with agents that inhibit spleen tyrosine kinase (Syk) and Btk, both components of the BCR signaling pathway.

United States published patent application number US 2010/0029610, published Feb. 4, 2010 (“the '610 publication,” the entirety of which is hereby incorporated herein by reference), describes certain 2,4-disubstituted pyrimidine compounds which covalently and irreversibly inhibit activity of one or more protein kinases, including BTK, a member of TEC-kinases. Such compounds include N-(3-(5-fluoro-2-(4-(2-methoxyethoxyl)phenylamino)pyrimidin-4-ylamino)phenyl)acrylamide, hereinafter referred to as Compound 1, which is designated as compound number 1-182 in the '610 publication. The synthesis of Compound 1 is described in detail at Example 20 of the '610 publication Compound 1 is active in a variety of assays and therapeutic models demonstrating covalent, irreversible inhibition of BTK (in enzymatic and cellular assays). Notably, Compound 1 is a potent, selective, orally available, small molecule which was found to inhibit B-cell proliferation and activation. Compound 1 is therefore useful for treating one or more disorders associated with activity of BTK.

Accordingly, among other things, the present invention provides methods of treating, stabilizing or lessening the severity or progression of one or more diseases and conditions associated with BTK. In some aspects, the present invention provides methods of treating, stabilizing or lessening the severity or progression of one or more diseases and conditions associated with BTK comprising administering to a patient in need thereof a pharmaceutically acceptable composition comprising N-(3-(5-fluoro-2-(4-(2-methoxyethoxyl)phenylamino)pyrimidin-4-ylamino)phenyl)acrylamide (1):

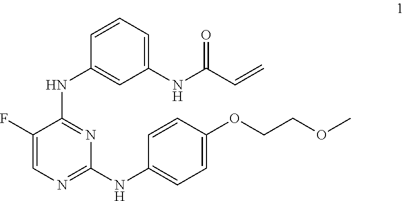 [SMILES: C=CC(=O)Nc1cccc(Nc2nc(Nc3ccc(OCCOC)cc3)ncc2F)c1]

In some embodiments, the present invention provides a method of treating, stabilizing or lessening the severity or progression of one or more diseases and conditions associated with BTK comprising administering to a patient in need thereof Compound 1 in combination with lenalidomide.

In some embodiments, the present invention provides a method of treating, stabilizing or lessening the severity or progression of one or more diseases and conditions associated with BTK comprising administering to a patient in need thereof a composition comprising Compound 1 in combination with a composition comprising lenalidomide.

In some embodiments, provided methods comprise administering to a patient in need thereof Compound 1 in combination with lenalidomide, wherein Compound 1 is administered once a day. In some embodiments, provided methods comprise administering to a patient in need thereof Compound 1 in combination with lenalidomide, wherein Compound 1 is administered twice a day. In some such embodiments, lenalidomide is administered once a day. Accordingly, in some embodiments, provided methods comprise administering to a patient in need thereof Compound 1 in combination with lenalidomide, wherein Compound 1 is administered twice a day and lenalidomide is administered once a day.

In some embodiments, the provided methods comprise administering to a patient in need thereof a composition comprising Compound 1 and lenalidomide.

In some embodiments, the disease or condition associated with BTK is selected from chronic lymphocytic leukemia and small lymphocytic lymphoma.

In some embodiments, the present invention provides a method of treating, stabilizing or lessening the severity or progression of chronic lymphocytic leukemia (CLL), the method comprising administering to a patient in need thereof Compound 1 in combination with lenalidomide.

In some embodiments, the present invention provides a method of treating, stabilizing or lessening the severity or progression of small lymphocytic lymphoma (SLL), the method comprising administering to a patient in need thereof Compound 1 in combination with lenalidomide.

In some embodiments, provided therapies comprise orally administering to a patient Compound 1 in combination with lenalidomide. In some embodiments, each of Compound 1 and lenalidomide is administered in the form of a pharmaceutical formulation. In some such embodiments, such formulations are capsule formulations.

In some embodiments, the present invention also provides dosing regimens and protocols for administering to patients in need thereof Compound 1 in combination with lenalidomide.

In some embodiments, the present invention provides a method of treating, stabilizing or lessening the severity or progression of one or more diseases and conditions associated with BTK comprising administering to a patient in need thereof each of Compound 1, lenalidomide and an anti-CD20 antibody (e.g., rituximab). In some such embodiments, the disease or condition associated with BTK is selected from chronic lymphocytic leukemia and small lymphocytic lymphoma.

Such methods, dosing regimens and protocols for the administration of said combination are described in further detail, below.

### BRIEF DESCRIPTION OF THE DRAWINGS

FIG. 1 presents a particular dose response curve for Compound 1 besylate in combination with lenalidomide (3000 nM) in the OCI-LY-10 cell line. =Compound 1 besylate; □=lenalidomide; Δ=calculated or expected activity of combination; ⋄=observed activity of the combination. Compound 1 besylate and lenalidomide appear to show an additive effect in the OCI-LY-10 cell line.

FIG. 2 presents a “volcano” plot of Compound 1 besylate (0.2 nM-1500 nM) in combination with lenalidomide (300 nM, 1000 nM and 3000 nM) in the OCI-LY-10 cell line. Compound 1 besylate and lenalidomide appear to show an additive effect in the OCI-LY-10 cell line.

FIG. 3 presents a particular dose response curve for Compound 1 besylate in combination with lenalidomide (3333 nM) in the WSU-DLCL2 cell line. =Compound 1 besylate; □=lenalidomide; Δ=calculated or expected activity of combination; ⋄=observed activity of the combination. Compound 1 besylate and lenalidomide appear to show an additive effect in the WSU-DLCL2 cell line.

FIG. 4 presents a “volcano” plot of Compound 1 besylate (0.5 nM-3333 nM) in combination with lenalidomide (123 nM, 3333 nM and 10000 nM) in the WSU-DLCL2 cell line. Compound 1 besylate and lenalidomide appear to show an additive effect in the WSU-DLCL2 cell line.

FIG. 5 presents a particular dose response curve for Compound 1 besylate in combination with lenalidomide (3333 nM) in the Riva cell line. =Compound 1 besylate; □=lenalidomide; Δ=calculated or expected activity of combination; ⋄=observed activity of the combination. Apparent synergistic effect is indicated by the arrow.

FIG. 6 presents a “volcano” plot of Compound 1 besylate (0.2 nM-1000 nM) in combination with lenalidomide (41 nM, 1111 nM and 3333 nM) in the Riva cell line. Apparent synergistic effect is indicated by the arrow.

FIG. 7 presents a particular dose response curve for Compound 1 besylate in combination with lenalidomide (333 nM) in the Riva cell line. =Compound 1 besylate; □=lenalidomide; Δ=calculated or expected activity of combination; ⋄=observed activity of the combination. Apparent synergy is indicated by the arrow.

FIG. 8 presents a “volcano” plot of Compound 1 besylate (0.2 nM-1000 nM) in combination with lenalidomide (41 nM, 123 nM and 370 nM) in the Riva cell line. Apparent synergy is indicated by the arrow.

FIG. 9 presents a particular dose response curve for Compound 1 besylate in combination with lenalidomide (333 nM) in the Riva cell line. =Compound 1 besylate; □=lenalidomide; Δ=calculated or expected activity of combination; ⋄=observed activity of the combination.

FIG. 10 presents a “volcano” plot of Compound 1 besylate (0.2 nM-1000 nM) in combination with lenalidomide (41 nM, 123 nM and 370 nM) in the Riva cell line. Compound 1 besylate and lenalidomide appear to show an additive effect in the Riva cell line.

FIG. 11 presents a particular dose response curve for Compound 1 besylate in combination with lenalidomide (3000 nM) in the TMD-8 cell line. =Compound 1 besylate; □=lenalidomide; Δ=calculated or expected activity of combination; ⋄=observed activity of the combination. Apparent synergy is indicated by the arrow.

FIG. 12 presents a “volcano” plot of Compound 1 besylate (0.2 nM-1500 nM) in combination with lenalidomide (300 nM, 1000 nM and 3000 nM) in the TMD-8 cell line. Apparent synergy is indicated by the arrow.

FIG. 13 presents the Response Assessments of patients enrolled in cohort 1 as of Oct. 16, 2013.

FIG. 14 presents the Response Assessments of patients enrolled in cohort 2 as of Oct. 16, 2013.

### DETAILED DESCRIPTION OF THE INVENTION

### Definitions

As used herein, the terms “combination”, “in combination with” or “combination therapy” refer to those situations in which two or more different pharmaceutical agents are administered in overlapping regimens so that the subject is simultaneously exposed to both agents. In some embodiments, such combinations refer to simultaneously administering to a subject separate dosage forms of Compound 1 and lenalidomide. In some embodiments, such combinations refer to contemporaneously administering to a subject separate dosage forms of Compound 1 and lenalidomide, wherein Compound 1 is administered before, during or after administration of lenalidomide. In some embodiments, simultaneous or contemporaneous exposure of Compound 1 and lenalidomide is effected via different dosage regimens appropriate for each therapeutic agent. For example, in some embodiments, Compound 1 is administered twice daily, or BID, whereas lenalidomide is administered once daily, or QD. Alternatively and/or additionally, Compound 1 may be administered once or twice daily for one or more 28-day cycles, whereas lenalidomide may be administered once daily for days 1 through 21 of one or more 28-day cycles. In some embodiments, Compound 1 is administered twice daily on days 8 through 28 of one or more 28-day cycles and lenalidomide is administered once daily on days 1 through 28 of one or more 28-day cycles. In some embodiments, Compound 1 is administered twice daily on days 1 through 28 of one or more 28-day cycles and lenalidomide is administered once daily on days 1 through 28 of one or more 28-day cycles.

The term “percent inhibition” as used herein refers to the percent decrease of target activity in the presence of a test compound (e.g., an irreversible BTK inhibitor) relative to control target activity. It will be appreciated that percent inhibition of a target (e.g., a kinase) can be determined in numerous ways, one of which is described in Example 3, infra. In some embodiments, percent inhibition is expressed as % inhibition (e.g., 50% inhibition). In some embodiments, the percent inhibition of a kinase is an average percent inhibition.

As used herein, the term “comparable”, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable. As used herein, the terms “comparable percent inhibition” or “comparable average percent inhibition” refer to a percent inhibition or an average percent inhibition, respectively, of a kinase that is within 10% of that observed or determined for a reference kinase inhibitor. For example, if a reference kinase inhibitor has 50% inhibition of a kinase relative to a control, another inhibitor will be considered to show comparable inhibition if it has about 40% to about 60% inhibition of the same kinase relative to the control. In some embodiments, an irreversible BTK inhibitor has comparable percent inhibition to a reference kinase inhibitor wherein the percent inhibition of the irreversible BTK inhibitor is within 9%, 8%, 7%, 6%, 5%, 4%, 3%, 2% or 1% inhibition of that observed or determined for a reference kinase inhibitor.

As used herein, a “disease or disorder associated with BTK” or a “BTK-mediated disorder” means any disease or other deleterious condition in which BTK, or a mutant thereof, is known or suspected to play a role. Accordingly, another embodiment of the present invention relates to preventing, treating, stabilizing or lessening the severity or progression of one or more diseases in which BTK, or a mutant thereof, is known or suspected to play a role. Specifically, the present invention relates to a method of treating or lessening the severity of a proliferative disorder, wherein said method comprises administering to a patient in need thereof Compound 1 in combination with lenalidomide.

As used herein, the term “irreversible” or “irreversible inhibitor” refers to an inhibitor (i.e. a compound) that is able to be covalently bonded to a target protein kinase in a substantially non-reversible manner. That is, whereas a reversible inhibitor is able to bind to (but is generally unable to form a covalent bond to) the target protein kinase, and therefore can become dissociated from the target protein kinase, an irreversible inhibitor will remain substantially bound to the target protein kinase once covalent bond formation has occurred. Irreversible inhibitors usually display time dependency, whereby the degree of inhibition increases with the time with which the inhibitor is in contact with the enzyme. Methods for identifying if a compound is acting as an irreversible inhibitor are known to one of ordinary skill in the art. Such methods include, but are not limited to, enzyme kinetic analysis of the inhibition profile of the compound with the protein kinase target, the use of mass spectrometry of the protein drug target modified in the presence of the inhibitor compound, discontinuous exposure, also known as “washout,” experiments, and the use of labeling, such as radiolabelled inhibitor, to show covalent modification of the enzyme, as well as other methods known to one of skill in the art.

The term “refractory CLL/SLL” as used herein is defined as CLL/SLL which was treated with at least one line of prior therapy (i) without achieving at least a partial response to therapy or (ii) which progressed within 6 months of treatment.

The term “relapsed CLL/SLL” as used herein is defined as CLL/SLL which progressed after ≧6 months post-treatment after achieving partial response or complete response to therapy.

The term “subject”, as used herein, means a mammal and includes human and animal subjects, such as domestic animals (e.g., horses, dogs, cats, etc.).

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a “therapeutically effective amount” is at least a minimal amount of a compound, or composition containing a compound, which is sufficient for treating one or more symptoms of a disorder or condition associated with Bruton's tyrosine kinase.

The terms “treat” or “treating,” as used herein, refers to partially or completely alleviating, inhibiting, delaying onset of, preventing, ameliorating and/or relieving a disorder or condition, or one or more symptoms of the disorder or condition. As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

The expression “unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

As used herein, the term “antibody”, or grammatical variations thereof (i.e., antibodies), refers to polypeptide(s) capable of binding to an epitope. In some embodiments, an antibody is a full-length antibody. In some embodiments, an antibody is less than full length (i.e., an antibody fragment) but includes at least one binding site. In some such embodiments, the binding site comprises at least one, and preferably at least two sequences with structure of antibody variable regions. In some embodiments, the term “antibody” encompasses any protein having a binding domain which is homologous or largely homologous to an immunoglobulin-binding domain. In particular embodiments, the term “antibody” encompasses polypeptides having a binding domain that shows at least 99% identity with an immunoglobulin-binding domain. In some embodiments, the antibody is any protein having a binding domain that shows at least 70%, at least 80%, at least 85%, at least 90% or at least 95% identity with an immunoglobulin-binding domain. Antibody polypeptides in accordance with the present invention may be prepared by any available means, including, for example, isolation from a natural source or antibody library, recombinant production in or with a host system, chemical synthesis, etc., or combinations thereof. In some embodiments, an antibody is monoclonal or polyclonal. In some embodiments, an antibody may be a member of any immunoglobulin class, including any of the human classes IgG, IgM, IgA, IgD and IgE. In certain embodiments, an antibody is a member of the IgG immunoglobulin class. In some embodiments, the term “antibody” refers to any derivative of an antibody that possesses the ability to bind to an epitope of interest. In some embodiments, an antibody fragment comprises multiple chains that are linked together, for example, by disulfide linkages. In some embodiments, an antibody is a human antibody. In some embodiments, an antibody is a humanized antibody. In some embodiments, humanized antibodies include chimeric immunoglobulins, immunoglobulin chains or antibody fragments (Fv, Fab, Fab′, F(ab′)₂ or other antigen binding subsequences of antibodies) that contain minimal sequence derived from non-human immunoglobulin. In some embodiments, humanized antibodies are human immunoglobulin (recipient antibody) in which residues from a complementary-determining region (CDR) of the recipient are replaced by residues from a CDR of a non-human species (donor antibody) such as mouse, rat or rabbit having the desired specificity, affinity and capacity. In particular embodiments, antibodies for use in the present invention bind to particular epitopes of CD20. In some embodiments, epitopes of CD20 to which anti-CD20 antibodies bind include, for example, ¹⁷⁰ANPS¹⁷³ (Binder et al., Blood 2006, 108(6): 1975-1978), FMC7 (Deans et al., Blood 2008, 111(4): 2492), Rp5-L and Rp15-C (mimotopes of CD20) (Perosa et al., J. Immunol. 2009, 182:416-423), ¹⁸²YCYSI¹⁸⁵ (Binder et al., Blood 2006, 108(6): 1975-1978) and WEWTI (a mimic of ¹⁸²YCYSI¹⁸⁵) (Binder et al., Blood 2006, 108(6): 1975-1978). In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

As used herein, the term “biosimilar” (for example, of an approved reference product/biological drug, such as a protein therapeutic, antibody, etc.) refers to a biologic product that is similar to the reference product based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is approved and intended to be used and for which approval is sought (e.g., that there are no clinically meaningful differences between the biological product and the reference product in terms of the safety, purity, and potency of the product).

In some embodiments, the biosimilar biological product and reference product utilizes the same mechanism or mechanisms of action for the condition or conditions of use prescribed, recommended, or suggested in the proposed labeling, but only to the extent the mechanism or mechanisms of action are known for the reference product. In some embodiments, the condition or conditions of use prescribed, recommended, or suggested in the labeling proposed for the biological product have been previously approved for the reference product. In some embodiments, the route of administration, the dosage form, and/or the strength of the biological product are the same as those of the reference product. In some embodiments, the facility in which the biological product is manufactured, processed, packed, or held meets standards designed to assure that the biological product continues to be safe, pure, and potent. The reference product may be approved in at least one of the U.S., Europe, or Japan. A biosimilar can be for example, a presently known antibody having the same primary amino acid sequence as a marketed antibody, but may be made in different cell types or by different production, purification or formulation methods.

### General Methods of Treating a BTK-Mediated Disease or Disorder

In some embodiments, the present invention provides a method of treating, stabilizing or lessening the severity or progression of one or more diseases and conditions associated with BTK comprising administering to a patient in need thereof an irreversible BTK inhibitor and lenalidomide.

It is understood that although the methods described herein refer to formulations, doses and dosing regimens/schedules of Compound 1 and salts thereof, such formulations, doses and/or dosing regimens/schedules are equally applicable to any irreversible BTK inhibitor, such as those described below. Accordingly, in some embodiments, a dose or dosing regimen of an irreversible BTK inhibitor is selected from any of the doses or dosing regimens for Compound 1 as described herein. In some embodiments, provided methods comprise administering an irreversible BTK inhibitor in an amount selected from any of the doses for Compound 1 as described herein. In some such embodiments, a dose of an irreversible BTK inhibitor is administered according to a dosing schedule selected from any of the dosing schedules described herein for Compound 1. In some embodiments, a composition comprising an irreversible BTK inhibitor is any of the formulations as described herein.

In some embodiments, the present invention provides a method of treating, stabilizing or lessening the severity or progression of one or more diseases and conditions associated with BTK comprising administering to a patient in need thereof an irreversible BTK inhibitor, lenalidomide and an anti-CD20 antibody (e.g., rituximab).

In some embodiments, the irreversible BTK inhibitor covalently binds to Cys 481 of BTK.

In some embodiments, an irreversible BTK inhibitor has activity against one or more kinases selected from the kinases recited in Table 7, infra.

In some embodiments, an irreversible BTK inhibitor has a percent inhibition comparable to that of a reference kinase inhibitor with respect to a kinase selected from Table 7, or combinations thereof. In some such embodiments, the reference kinase inhibitor is Compound 2:

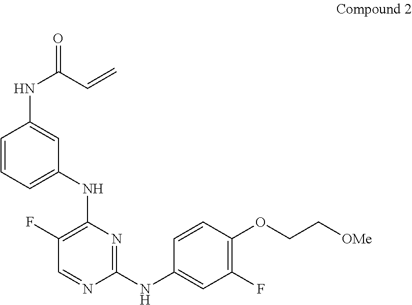 [SMILES: C=CC(=O)Nc1cccc(Nc2nc(Nc3ccc(OCCOC)c(F)c3)ncc2F)c1]

In some embodiments, the percent inhibition of the reference kinase inhibitor is that shown for Compound 2 in Example 3.

In some embodiments, an irreversible BTK inhibitor has a percent inhibition comparable to that of Compound 2 with respect to one or more kinases selected from Table 7, or combinations thereof, in that the irreversible kinase inhibitor has a percent inhibition within approximately 10% of that observed for Compound 2. In some embodiments, an irreversible BTK inhibitor has a percent inhibition comparable to that of Compound 2 with respect to one or more kinases selected from Table 7, or combinations thereof, in that the irreversible kinase inhibitor has a percent inhibition that is within about 9%, or about 8%, or about 7%, or about 6%, or about 5%, or about 4%, or about 3%, or about 2% or about 1% inhibition of that observed for Compound 2.

In some embodiments, an irreversible BTK inhibitor has a percent inhibition that is greater than that observed for Compound 2 with respect to one or more kinases selected from Table 7. In some embodiments, an irreversible BTK inhibitor has a percent inhibition that is less than that observed for Compound 2 with respect to one or more kinases selected from Table 7.

In some embodiments, the irreversible BTK inhibitor has a percent inhibition of one or more additional kinases, wherein the percent inhibition of the kinase or kinases is at least about 50%, at least about 55%, at least about 60%, at least about 65%, at least about 70%, at least about 75%, at least about 80%, at least about 85%, at least about 90% or at least about 95%.

In some embodiments, the irreversible BTK inhibitor has a percent inhibition comparable to that of a reference kinase inhibitor with respect to one or more kinases selected from the group consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YESNES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YES/YES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor inhibits a kinase selected from the group consisting of TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YESNES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof, wherein the inhibition of the kinase or kinases is at least the percent inhibition observed for a reference kinase inhibitor. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least 65%, at least about 70%, at least about 75%, at least about 80%, at least about 85%, at least about 90% or at least about 95% inhibition of a kinase selected from TXK, BMX/ETK, FLT3, BLK, TEC, ERBB4/HER4, Aurora B, TRKC, RET, LOK/STK10, Aurora C, FLT4/VEGFR3, ROS/ROS1, ARK5/NUAK1, EGFR, DDR1, JAK3, LRRK2, ABL2/ARG, ITK, Aurora A, YES/YES1, FGFR3, TNK1, BRK, FGFR2, PDGFRb, c-SRC, ACK1, FGFR1, STK16, ABL1, AXL, TYK2, ERBB2/HER2, FGR, CHK2, SIK1, MLK1/MAP3K9, MLK2/MAP3K10, MLK3/MAP3K11, PKCb2 and CLK2, or combinations thereof.

In some embodiments, the irreversible BTK inhibitor has a percent inhibition comparable to that of a reference kinase inhibitor with respect to one or more kinases selected from the group consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is at least the percent inhibition observed for a reference kinase inhibitor with respect to one or more kinases selected from the group consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least about 65%, at least about 70%, at least about 75%, at least about 80%, at least about 85%, at least about 90% or at least about 95% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof.

In some embodiments, the present invention provides a method of treating, stabilizing or lessening the severity or progression of CLL/SLL comprising administering to a patient in need thereof an irreversible BTK inhibitor and lenalidomide, wherein the irreversible BTK inhibitor has not more than about 50% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2, or combinations thereof.

In some embodiments, the irreversible BTK inhibitor has at least about 50% inhibition of a kinase selected from Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11 and PKCb2, or combinations thereof. In some embodiments, the irreversible BTK inhibitor has has at least about 50% inhibition of the group of kinases consisting of Aurora A, Aurora B, Aurora C, TRKC, ROS/ROS1, ARK5/NUAK1, LRRK2, ABL2/ARG, TNK1, STK16, ABL1, AXL, TYK2, CHK2, SIK1, MLK1/MAP3K9, MLK2/MAP3K10, MLK3/MAP3K11, PKCb2 and CLK2.

In some embodiments, the irreversible BTK inhibitor has at least about 55% inhibition of a kinase selected from Aurora A, Aurora B, Aurora C, TRKC, ROS/ROS1, ARK5/NUAK1, LRRK2, ABL2/ARG, TNK1, STK16, ABL1, CHK2, MLK1/MAP3K9, MLK2/MAP3K10 and MLK3/MAP3K11, or combinations thereof. In some embodiments, the irreversible BTK inhibitor has at least about 55% inhibition of the group of kinases consisting of Aurora A, Aurora B, Aurora C, TRKC, ROS/ROS1, ARK5/NUAK1, LRRK2, ABL2/ARG, TNK1, STK16, ABL1, CHK2, MLK1/MAP3K9, MLK2/MAP3K10 and MLK3/MAP3K11.

In some embodiments, the irreversible BTK inhibitor has at least about 60% inhibition of a kinase selected from Aurora A, Aurora B, Aurora C, TRKC, ROS/ROS1, ARK5/NUAK1, LRRK2, ABL2/ARG, TNK1, STK16, CHK2, MLK1/MAP3K9 and MLK3/MAP3K11, or combinations thereof. In some embodiments, the irreversible BTK inhibitor has at least about 60% inhibition of the group of kinases consisting of Aurora A, Aurora B, Aurora C, TRKC, ROS/ROS1, ARK5/NUAK1, LRRK2, ABL2/ARG, TNK1, STK16, CHK2, MLK1/MAP3K9 and MLK3/MAP3K11.

In some embodiments, the irreversible BTK inhibitor has at least about 65% inhibition of a kinase selected from Aurora A, Aurora B, Aurora C, TRKC, ROS/ROS1, ARK5/NUAK1, LRRK2, TNK1, STK16, CHK2, MLK1/MAP3K9 and MLK3/MAP3K11, or combinations thereof. In some embodiments, the irreversible BTK inhibitor sh at least about 65% inhibition of the group of kinases consisting of Aurora A, Aurora B, Aurora C, TRKC, ROS/ROS1, ARK5/NUAK1, LRRK2, TNK1, STK16, CHK2, MLK1/MAP3K9 and MLK3/MAP3K11.

In some embodiments, the irreversible BTK inhibitor has at least about 70% inhibition of a kinase selected from Aurora A, Aurora B, Aurora C, ROS/ROS1, ARK5/NUAK1, TNK1, STK16, CHK2, MLK1/MAP3K9 and MLK3/MAP3K11, or combinations thereof. In some embodiments, the irreversible BTK inhibitor at least about 70% inhibition of the group of kinases consisting of Aurora A, Aurora B, Aurora C, ROS/ROS1, ARK5/NUAK1, TNK1, STK16, CHK2, MLK1/MAP3K9 and MLK3/MAP3K11.

In some embodiments, the irreversible BTK inhibitor has at least about 75% inhibition of a kinase selected from Aurora A, Aurora B, ROS/ROS1, ARK5/NUAK1, TNK1, STK16 and MLK1/MAP3K9, or combinations thereof. In some embodiments, the irreversible BTK inhibitor has at least about 75% inhibition of the group of kinases consisting of Aurora A, Aurora B, ROS/ROS1, ARK5/NUAK1, TNK1, STK16 and MLK1/MAP3K9.

In some embodiments, the irreversible BTK inhibitor has at least about 80% inhibition of a kinase selected from Aurora A, Aurora B, ROS/ROS1, ARK5/NUAK1, TNK1 and MLK1/MAP3K9, or combinations thereof. In some embodiments, the irreversible BTK inhibitor has at least about 80% inhibition of the group of kinases consisting of Aurora A, Aurora B, ROS/ROS1, ARK5/NUAK1, TNK1 and MLK1/MAP3K9.

In some embodiments, the irreversible BTK inhibitor has at least about 85% inhibition of a kinase selected from Aurora A, Aurora B, ROS/ROS1, ARK5/NUAK1 and MLK1/MAP3K9, or combinations thereof. In some embodiments, the irreversible BTK inhibitor has at least about 85% inhibition of the group of kinases consisting of Aurora A, Aurora B, ROS/ROS1, ARK5/NUAK1 and MLK1/MAP3K9.

In some embodiments, the irreversible BTK inhibitor has a percent inhibition comparable to that of a reference kinase inhibitor with respect to one or more kinases selected from the group consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is at least the percent inhibition observed for a reference kinase inhibitor with respect to one or more kinases selected from the group consisting of TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has at least about 50%, at least about 55%, at least about 60%, at least about 65%, at least about 70%, at least about 75%, at least about 80%, at least about 85%, at least about 90% or at least about 95% inhibition of a kinase selected from TNK1, STK16, ABL1, AXL, TYK2, CHK2, MLK1/MAP3K9, MLK2/MAP3K10, MLK3/MAP3K11, SIK1, PKCb2 and CLK2, or combinations thereof.

In some embodiments, the irreversible BTK inhibitor has at least about 50% inhibition of a kinase selected from TNK1, STK16, ABL1, AXL, TYK2, CHK2, MLK1/MAP3K9, MLK2/MAP3K10, MLK3/MAP3K11, SIK1, PKCb2 and CLK2, or combinations thereof. In some embodiments, the irreversible BTK inhibitor at least about 50% inhibition of the group of kinases consisting of TNK1, STK16, ABL1, AXL, TYK2, CHK2, MLK1/MAP3K9, MLK2/MAP3K10, MLK3/MAP3K11, SIK1, PKCb2 and CLK2.

In some embodiments, the irreversible BTK inhibitor has at least about 55% inhibition of a kinase selected from TNK1, STK16, ABL1, CHK2, MLK1/MAP3K9 and MLK3/MAP3K11, or combinations thereof. In some embodiments, the irreversible BTK inhibitor has at least about 55% inhibition of the group of kinases consisting of TNK1, STK16, ABL1, CHK2, MLK1/MAP3K9 and MLK3/MAP3K11, or combinations thereof.

In some embodiments, the irreversible BTK inhibitor has at least about 60%, at least about 65% or at least about 70% inhibition of a kinase selected from TNK1, STK16, CHK2, MLK1/MAP3K9 and MLK3/MAP3K11, or combinations thereof. In some embodiments, the irreversible BTK inhibitor has at least about 60%, at least about 65% or at least about 70% inhibition of the group of kinases consisting of CHK2, MLK1/MAP3K9 and MLK3/MAP3K11.

In some embodiments, the irreversible BTK inhibitor has at least about 75% inhibition of a kinase selected from TNK1, STK16 and MLK1/MAP3K9, or combinations thereof. In some embodiments, the irreversible BTK inhibitor has at least about 75% inhibition of the the group of kinases consisting of TNK1, STK16 and MLK1/MAP3K9.

In some embodiments, an irreversible BTK inhibitor for use in the present invention has a percent inhibition comparable to that of a reference kinase inhibitor with respect to one or more kinases selected from the group consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition comparable to that of a reference kinase inhibitor with respect to the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition that is not more than the percent inhibition observed for a reference kinase inhibitor with respect to one or more kinases selected from the group consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from RIPK2, HCK, LYN, CSK, LCK, LYN B and FYN, or combinations thereof.

In some embodiments, the irreversible BTK inhibitor has not more than about 50%, not more than about 45%, not more than about 40%, not more than about 35%, not more than about 30%, not more than about 25%, not more than about 20%, not more than about 15%, not more than about 10% or not more than about 5% inhibition of a kinase selected from EPHA6, LYN B, FRK/PTK5, RIPK3, BRAF, ARAF and SRMS, or combinations thereof.

In some embodiments, the present invention provides a method of treating, stabilizing or lessening the severity or progression of CLL/SLL comprising administering to a patient in need thereof an irreversible BTK inhibitor and lenalidomide, wherein the irreversible BTK inhibitor has at least about 50% inhibition of a kinase selected from c-Kit, PDGFRa, RIPK2, HCK, EPHA6, LYN, CSK, LCK, ZAK/MLTK, LYN B, FRK/PTK5, FYN, BRAF, RIPK3, ARAF and SRMS, or combinations thereof.

In some embodiments, the irreversible BTK inhibitor has not more than about 50% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 50% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 45% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 45% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 40% inhibition of a kinase selected from c-Kit, PDGFRa, RIPK2, HCK, EPHA6, LYN, CSK, LCK, ZAK/MLTK, LYN B, FRK/PTK5, FYN, RIPK3, BRAF, ARAF and SRMS, or combinations thereof. In some embodiments, the irreversible BTK inhibitor has not more than about 40% inhibition of the group of kinases consisting of c-Kit, PDGFRa, RIPK2, HCK, EPHA6, LYN, CSK, LCK, ZAK/MLTK, LYN B, FRK/PTK5, FYN, RIPK3, BRAF, ARAF and SRMS.

In some embodiments, the irreversible BTK inhibitor has not more than about 35% inhibition of a kinase selected from c-Kit, RIPK2, HCK, EPHA6, LYN, CSK, ZAK/MLTK, LYN B, FRK/PTK5, FYN, RIPK3, BRAF, ARAF and SRMS, or combinations thereof. In some embodiments, the irreversible BTK inhibitor has not more than about 35% inhibition of the group of kinases consisting of c-Kit, RIPK2, HCK, EPHA6, LYN, CSK, ZAK/MLTK, LYN B, FRK/PTK5, FYN, RIPK3, BRAF, ARAF and SRMS.

In some embodiments, the irreversible BTK inhibitor has not more than about 30% inhibition of a kinase selected from c-Kit, RIPK2, HCK, EPHA6, LYN, CSK, ZAK/MLTK, LYN B, FRK/PTK5, FYN, RIPK3, BRAF, ARAF and SRMS, or combinations thereof. In some embodiments, the irreversible BTK inhibitor has not more than about 30% inhibition of the group of kinases consisting of c-Kit, RIPK2, HCK, EPHA6, LYN, CSK, ZAK/MLTK, LYN B, FRK/PTK5, FYN, RIPK3, BRAF, ARAF and SRMS.

In some embodiments, the irreversible BTK inhibitor has not more than about 25% inhibition of a kinase selected from c-Kit, RIPK2, EPHA6, CSK, ZAK/MLTK, LYN B, FRK/PTK5, FYN, RIPK3, BRAF, ARAF and SRMS, or combinations thereof. In some embodiments, the irreversible BTK inhibitor has not more than about 25% inhibition of the group of kinases consisting of c-Kit, IPK2, EPHA6, CSK, ZAK/MLTK, LYN B, FRK/PTK5, FYN, RIPK3, BRAF, ARAF and SRMS.

In some embodiments, the irreversible BTK inhibitor has not more than about 20% inhibition of a kinase selected from EPHA6, CSK, ZAK/MLTK, LYN B, FRK/PTK5, FYN, RIPK3, BRAF, ARAF and SRMS, or combinations thereof. In some embodiments, the irreversible BTK inhibitor has not more than about 20% inhibition of the group of kinases consisting of EPHA6, CSK, ZAK/MLTK, LYN B, FRK/PTK5, FYN, RIPK3, BRAF, ARAF and SRMS.

In some embodiments, the irreversible BTK inhibitor has not more than about 15% inhibition of a kinase selected from EPHA6, LYN B, FRK/PTK5, RIPK3, BRAF, ARAF and SRMS, or combinations thereof. In some embodiments, the irreversible BTK inhibitor has not more than about 15% inhibition of the group of kinases consisting of EPHA6, LYN B, FRK/PTK5, RIPK3, BRAF, ARAF and SRMS.

In some embodiments, the irreversible BTK inhibitor has not more than about 10% inhibition of a kinase selected from EPHA6, LYN B, FRK/PTK5, RIPK3, BRAF, ARAF and SRMS, or combinations thereof. In some embodiments, the irreversible BTK inhibitor has not more than about 10% inhibition of the group of kinases consisting of EPHA6, LYN B, FRK/PTK5, RIPK3, BRAF, ARAF and SRMS.

In some embodiments, the irreversible BTK inhibitor has not more than about 5% inhibition of a kinase selected from EPHA6, FRK/PTK5, RIPK3, BRAF, ARAF and SRMS, or combinations thereof. In some embodiments, the irreversible BTK inhibitor has not more than about 5% inhibition of the group of kinases consisting of EPHA6, FRK/PTK5, RIPK3, BRAF, ARAF and SRMS.

In some embodiments, the irreversible BTK inhibitor has a percent inhibition of LYN comparable to that of a reference kinase inhibitor. In some embodiments, the irreversible BTK inhibitor has a percent inhibition of LYN that is not more than that observed for a reference kinase inhibitor. In some embodiments, the irreversible BTK inhibitor has a percent inhibition of LYN that is about 20-30%. In some embodiments, the irreversible BTK inhibitor has a percent inhibition of LYN that is about 25-30%. In some embodiments, the irreversible BTK inhibitor has a percent inhibition of LYN that is about 25-28%. In some embodiments, the irreversible BTK inhibitor has a percent inhibition of LYN that is not more than about 25%, not more than about 26%, not more than about 27%, not more than about 28%, not more than about 29%, not more than about 30%, not more than about 31%, not more than about 32% or not more than about 33%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of c-Kit comparable to that of a reference kinase inhibitor. In some embodiments, the irreversible BTK inhibitor has a percent inhibition of c-Kit that is not more than that observed for a reference kinase inhibitor. In some embodiments, the irreversible BTK inhibitor has a percent inhibition of c-Kit that is about 15-25%. In some embodiments, the irreversible BTK inhibitor has a percent inhibition of c-Kit that is about 20-25%. In some embodiments, the irreversible BTK inhibitor has a percent inhibition of c-Kit that is about 20-23%. In some embodiments, the irreversible BTK inhibitor has a percent inhibition of c-Kit that is not more than about 15%, not more than about 16%, not more than about 17%, not more than about 18%, not more than about 19%, not more than about 20%, not more than about 21%, not more than about 22%, not more than about 23%, not more than about 24% or not more than about 25%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of PDGFRa comparable to that of a reference kinase inhibitor. In some embodiments, the irreversible BTK inhibitor has a percent inhibition of PDGFRa that is not more than that observed for a reference kinase inhibitor. In some embodiments, the irreversible BTK inhibitor has a percent inhibition of PDGFRa that is about 30-40%. In some embodiments, the irreversible BTK inhibitor has a percent inhibition of PDGFRa that is about 35-40%. In some embodiments, the irreversible BTK inhibitor has a percent inhibition of PDGFRa that is about 35-38%. In some embodiments, the irreversible BTK inhibitor has a percent inhibition of PDGFRa that is not more than about 30%, not more than about 31%, not more than about 32%, not more than about 33%, not more than about 34%, not more than about 35%, not more than about 36%, not more than about 37%, not more than about 38%, not more than about 39% or not more than about 40%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of RIPK2 comparable to that of a reference kinase inhibitor. In some embodiments, the irreversible BTK inhibitor has a percent inhibition of RIPK2 that is not more than that observed for a reference kinase inhibitor. In some embodiments, the irreversible BTK inhibitor has a percent inhibition of RIPK2 that is about 20-30%. In some embodiments, the irreversible BTK inhibitor has a percent inhibition of RIPK2 that is about 20-25%. In some embodiments, the irreversible BTK inhibitor has a percent inhibition of RIPK2 that is about 22-25%. In some embodiments, the irreversible BTK inhibitor has a percent inhibition of RIPK2 that is not more than about 18%, not more than about 19%, not more than about 20%, not more than about 21%, not more than about 22%, not more than about 23%, not more than about 24%, not more than about 25%, not more than about 26% or not more than about 27%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of HCK comparable to that of a reference kinase inhibitor. In some embodiments, the irreversible BTK inhibitor has a percent inhibition of HCK that is not more than that observed for a reference kinase inhibitor. In some embodiments, the irreversible BTK inhibitor has a percent inhibition of HCK that is about 25-35%. In some embodiments, the irreversible BTK inhibitor has a percent inhibition of HCK that is about 27-32%. In some embodiments, the irreversible BTK inhibitor has a percent inhibition of HCK that is about 28-31%. In some embodiments, the irreversible BTK inhibitor has a percent inhibition of HCK that is not more than about 26%, not more than about 27%, not more than about 28%, not more than about 29%, not more than about 30%, not more than about 31%, not more than about 32%, not more than about 33% or not more than about 34%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of EPHA6 comparable to that of a reference kinase inhibitor. In some embodiments, the irreversible BTK inhibitor has a percent inhibition of EPHA6 that is not more than that observed for a reference kinase inhibitor. In some embodiments, the irreversible BTK inhibitor has a percent inhibition of EPHA6 that is about 0-10%. In some embodiments, the irreversible BTK inhibitor has a percent inhibition of EPHA6 that is about 0-5%. In some embodiments, the irreversible BTK inhibitor has a percent inhibition of EPHA6 that is about 0-3%. In some embodiments, the irreversible BTK inhibitor has a percent inhibition of EPHA6 that is not more than about 0.5%, not more than about 0.6%, not more than about 0.7%, not more than about 0.8%, not more than about 0.9%, not more than about 1%, not more than about 2%, not more than about 3% or not more than about 4%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of CSK comparable to that of a reference kinase inhibitor. In some embodiments, the irreversible BTK inhibitor has a percent inhibition of CSK that is not more than that observed for a reference kinase inhibitor. In some embodiments, the irreversible BTK inhibitor has a percent inhibition of CSK that is about 10-20%. In some embodiments, the irreversible BTK inhibitor has a percent inhibition of CSK that is about 15-20%. In some embodiments, the irreversible BTK inhibitor has a percent inhibition of CSK that is about 16-19%. In some embodiments, the irreversible BTK inhibitor has a percent inhibition of CSK that is not more than about 15%, not more than about 16%, not more than about 17%, not more than about 18%, not more than about 19%, not more than about 20%, not more than about 21%, not more than about 22% or not more than about 23%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of LCK comparable to that of a reference kinase inhibitor. In some embodiments, the irreversible BTK inhibitor has a percent inhibition of LCK that is not more than that observed for a reference kinase inhibitor. In some embodiments, the irreversible BTK inhibitor has a percent inhibition of LCK that is about 30-40%. In some embodiments, the irreversible BTK inhibitor has a percent inhibition of LCK that is about 32-37%. In some embodiments, the irreversible BTK inhibitor has a percent inhibition of LCK that is about 34-37%. In some embodiments, the irreversible BTK inhibitor has a percent inhibition of LCK that is not more than about 34%, not more than about 35%, not more than about 36%, not more than about 37%, not more than about 38%, not more than about 39%, not more than about 40%, not more than about 41% or not more than about 42%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of ZAK/MLTK comparable to that of a reference kinase inhibitor. In some embodiments, the irreversible BTK inhibitor has a percent inhibition of ZAK/MLTK that is not more than that observed for a reference kinase inhibitor. In some embodiments, the irreversible BTK inhibitor has a percent inhibition of ZAK/MLTK that is about 10-20%. In some embodiments, the irreversible BTK inhibitor has a percent inhibition of ZAK/MLTK that is about 12-17%. In some embodiments, the irreversible BTK inhibitor has a percent inhibition of ZAK/MLTK that is about 14-17%. In some embodiments, the irreversible BTK inhibitor has a percent inhibition of ZAK/MLTK that is not more than about 12%, not more than about 13%, not more than about 14%, not more than about 15%, not more than about 16%, not more than about 17%, not more than about 18%, not more than about 19% or not more than about 20%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of LYN B comparable to that of a reference kinase inhibitor. In some embodiments, the irreversible BTK inhibitor has a percent inhibition of LYN B that is not more than that observed for a reference kinase inhibitor. In some embodiments, the irreversible BTK inhibitor has a percent inhibition of LYN B that is about 0-10%. In some embodiments, the irreversible BTK inhibitor has a percent inhibition of LYN B that is about 3-8%. In some embodiments, the irreversible BTK inhibitor has a percent inhibition of LYN B that is about 4-7%. In some embodiments, the irreversible BTK inhibitor has a percent inhibition of LYN B that is not more than about 1%, not more than about 2%, not more than about 3%, not more than about 4%, not more than about 5%, not more than about 6%, not more than about 7%, not more than about 8%, not more than about 9% or not more than about 10%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of FRK/PTK5 comparable to that of a reference kinase inhibitor. In some embodiments, the irreversible BTK inhibitor has a percent inhibition of FRK/PTK5 that is not more than that observed for a reference kinase inhibitor. In some embodiments, the irreversible BTK inhibitor has a percent inhibition of FRK/PTK5 that is about 0-10%. In some embodiments, the irreversible BTK inhibitor has a percent inhibition of FRK/PTK5 that is about 0-5%. In some embodiments, the irreversible BTK inhibitor has a percent inhibition of FRK/PTK5 that is about 0-3%. In some embodiments, the irreversible BTK inhibitor has a percent inhibition of FRK/PTK5 that is not more than about 0.5%, not more than about 0.6%, not more than about 0.7%, not more than about 0.8%, not more than about 0.9%, not more than about 1%, not more than about 1.5%, not more than about 2%, not more than about 3% or not more than about 4%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of FYN comparable to that of a reference kinase inhibitor. In some embodiments, the irreversible BTK inhibitor has a percent inhibition of FYN that is not more than that observed for a reference kinase inhibitor. In some embodiments, the irreversible BTK inhibitor has a percent inhibition of FYN that is about 15-25%. In some embodiments, the irreversible BTK inhibitor has a percent inhibition of FYN that is about 15-20%. In some embodiments, the irreversible BTK inhibitor has a percent inhibition of FYN that is about 17-20%. In some embodiments, the irreversible BTK inhibitor has a percent inhibition of FYN that is not more than about 15%, not more than about 16%, not more than about 17%, not more than about 18%, not more than about 19%, not more than about 20%, not more than about 21%, not more than about 22% or not more than about 23%. In some embodiments, the reference kinase inhibitor is Compound 2. In some embodiments, the percent inhibition of the reference kinase inhibitor is that shown for Compound 2 in Example 3.

In some embodiments, the irreversible BTK inhibitor has a percent inhibition of BRAF comparable to that of a reference kinase inhibitor. In some embodiments, the irreversible BTK inhibitor has a percent inhibition of BRAF that is not more than that observed for a reference kinase inhibitor. In some embodiments, the irreversible BTK inhibitor has a percent inhibition of BRAF that is about 0-10%. In some embodiments, the irreversible BTK inhibitor has a percent inhibition of BRAF that is about 0.1-5%. In some embodiments, the irreversible BTK inhibitor has a percent inhibition of BRAF that is about 0.2-3%. In some embodiments, the irreversible BTK inhibitor has a percent inhibition of BRAF that is not more than about 0.1%, not more than about 0.2%, not more than about 0.3%, not more than about 0.4%, not more than about 0.5%, not more than about 0.6%, not more than about 0.7%, not more than about 0.8%, not more than about 0.9%, not more than about 1%, not more than about 2%, not more than about 3%, not more than about 4% or not more than about 5%. In some embodiments, the reference kinase inhibitor is Compound 2. In some embodiments, the percent inhibition of the reference kinase inhibitor is that shown for Compound 2 in Example 3.

Compound 1 is an Irreversible BTK Inhibitor

As described above, Bruton's tyrosine kinase (Btk) is a non-receptor tyrosine kinase with restricted cellular expression largely limited to B-lymphocytes, monocytes, and mast cells or basophils. Btk is a critical component of the B-cell receptor (BCR) signaling network and is crucial for B-cell development. Investigation has revealed that some B-cell malignancies, including CLL/SLL, depend on BCR signaling, suggesting that interruption of such signaling could be a promising therapeutic opportunity. Recently, clinical anti-tumor responses in various B-cell Non-Hodgkin's Lymphoma (NHL) and CLL/SLL have been reported with agents that inhibit spleen tyrosine kinase (Syk) and Btk, both components of the BCR signaling pathway.

Compound 1 is active in a variety of assays and therapeutic models demonstrating covalent, irreversible inhibition of BTK (in enzymatic and cellular assays). Compound 1 inhibits Btk activity by binding with high affinity to the adenosine triphosphate (ATP) binding site of Btk and forming a targeted covalent bond with the Btk protein, providing rapid, complete, and prolonged inhibition of Btk activity, both in vitro and in vivo.

Phosphorylation of the auto-phosphorylation site on Btk (Tyr223) and the Btk responsive site (Tyr1217) on PLCγ2 in Ramos cells, a human Burkitt lymphoma cell line, was inhibited by Compound 1 with an effective concentration required for 50% inhibition (EC₅₀) of 1 nM to 10 nM. Compound 1 demonstrates a high degree of selectivity in cellular assay systems against related kinases.

In single dose studies in healthy subjects, Compound 1 evidenced adequate safety, predictable pharmacokinetics (PK), and, at doses greater than 0.5 mg/kg, 80% to 100% occupancy of the Btk receptor target in normal human peripheral blood B-cells. A phase I dose escalation study of a single agent of Compound 1 is currently being conducted in different hematologic malignancies, including CLL/SLL.

Lenalidomide

Lenalidomide (REVLIMID®) belongs to the class of pharmaceutical compounds known as immunomodulatory drugs (IMiDsP®). The key to its therapeutic potential lies in the fact that it has multiple mechanisms of action, which act to produce both anti-inflammatory and antitumor effects. These effects depend on both the cell type and the triggering stimulus. To date, lenalidomide has been associated with tumor necrosis factor (TNF)-α inhibitory, T cell co-stimulatory, anti-proliferative and anti-angiogenic activities.

Lenalidomide has been approved by multiple global Health Authorities (including the United States Food and Drug Administration, but excluding the European Union) for the treatment of patients with transfusion dependent anemia due to Low- or Intermediate-1-risk myelodysplastic syndrome associated with a deletion 5q cytogenetic abnormality with or without other cytogenetic abnormalities. Lenalidomide has also been approved by multiple global Health Authorities (notably including the US and EU) in combination with dexamethasone, for patients with previously treated multiple myeloma (MM).

Lenalidomide is being investigated as treatment for various oncologic indications, including MM, NHL, and solid tumors. Studies have also been conducted in non-oncologic indications.

Tumor necrosis factor-α has been proposed to be an autocrine growth factor through the TNF-α receptor on CLL cells. High serum vascular endothelial growth factor (VEGF) levels and vascular endothelial growth factor receptor 1 (VEGFR-1) and vascular endothelial growth factor receptor 2 (VEGFR-2) expression on CLL cells have also been reported. Lenalidomide has been demonstrated to have the ability to modulate and regulate growth factors/cytokines, including VEGF, TNF-α, interleukin (IL)-10 and IL-6. Immunomodulatory drugs have been demonstrated to co-stimulate immune effector cells (T and natural killer cells). These observations and the known activity of thalidomide and lenalidomide in another B-cell malignancy, multiple myeloma, provide the initial rationale to study lenalidomide in CLL/SLL.

Single-agent lenalidomide has been studied in relapsed/refractory CLL. Subjects were treated with lenalidomide 25 mg for 21 days of every 28-day cycle. The overall response was 53% (18% Complete Response [CR], 36% Partial Response [PR]). Clinical responses (CR or PR) were observed in 47% of the subjects with del (11)(q23) and/or del (17)(p13.1) adverse cytogenetics and in 30% of subjects refractory to fludarabine. The median time to best response was 5.9 months (1.6-18.3 months) and the median progression-free survival (PFS) time was 19.4 months (1.2 to 31.8 months). Further, subjects with relapsed/refractory CLL were treated with lenalidomide administered daily in 28-day cycles up to disease progression.

Substantial clinical data indicate that lenalidomide has activity in relapsed/refractory aggressive B-cell malignancies. Furthermore, the B-cell receptor (BCR) pathway, of which Btk is a downstream component, has been implicated in the pathogenesis of B-cell proliferation, activation and survival. The role of Btk in CLL/SLL is supported by early reports of clinical studies involving Btk inhibitors.

Anti-CD20 Antibodies

CD20, the first B-cell specific antigen defined by the monoclonal antibody tositumomab, plays a critical role in B-cell development. Human CD20 is a 297 amino acid (30- to 35-kDa) phosphoprotein with four transmembrane domains encoded by the gene MS4A1 located on chromosome 11q12.2. CD20 plays a critical role in B-cell development and is a biomarker for immunotherapies targeting B-cell derived diseases. CD20 is an integral membrane protein expressed by B lymphocytes in early stages of differentiation and by most B cell lymphomas, but not by differentiated plasma cells. CD20 remains on the membrane of B cells without dissociation or internalization upon antibody binding. CD20 functions though binding to the Src family of tyrosine kinases, such as Lyn, Fyn and Lck, and believed to be involved as a result in the phosphorylation cascade of intracellular proteins. Anti-CD20 antibodies are broadly classified into type I and type II antibodies. Both types of anti-CD 20 antibodies exhibit equal ability in activating Fc-FcγR interactions such as antibody-dependent cellular cytotoxicity (ADCC) and phagocytosis. Type I anti-CD20 antibodies redistribute CD20 into membrane lipid rafts and potently activate complement-dependent cytotoxicity (CDC). Type II anti-CD20 antibodies weakly activate CDC but more potently induce direct programmed cell death.

In some embodiments, the present invention encompasses the recognition that a BTK inhibitor, e.g., Compound 1, together with lenalidomide and an anti-CD20 antibody, is useful in treating BTK-mediated diseases or disorders. Accordingly, in some embodiments, the present invention comprises a method of treating a BTK-mediated disease or disorder, the method comprising administering to a patient in need thereof each of Compound 1, lenalidomide and an anti-CD20 antibody. A person of ordinary skill in the art can readily identify and select additional anti-CD20 antibodies that are useful in the present invention. For example, in some embodiments, such antibodies are described, for example, in U.S. Pat. Nos. 8,153,125, 8,147,832, 8,101,179, 8,084,582, 8,057,793 and 7,879,984, and U.S. Patent Publication Nos. 2011/0129412, 2012/0183545, 2012/0134990 and 2012/0034185.

In some embodiments, an anti-CD20 antibody for use in the present invention is a type I antibody. In some embodiments, an anti-CD20 for use in the present invention is a type II antibody.

In some embodiments, an anti-CD20 antibody is an antibody that binds to a CD20 epitope selected from ¹⁷⁰ANPS¹⁷³ and ¹⁸²YCYSI¹⁸⁵.

In some embodiments, an anti-CD20 antibody has a binding affinity (K_{d}) for an epitope of CD20 of less than 12 nM, less than 11 nM, less than 10 nM, less than 9 nM, less than 8 nM, less than 7 nM, less than 6 nM, less than 5 nM, less than 4 nM, less than 3 nM, less than 2 nM or less than 1 nM.

Rituximab is but one example of an anti-CD20 antibody. In some embodiments, an anti-CD20 antibody for use in the present invention includes, for example, rituximab (Rituxan® or MabThera®), Gazyva® (i.e., obinutuzumab) and Arzerra® (ofatumumab). For ease of reference, provided methods and regimens detailed herein refer to an exemplary anti-CD20 antibody (i.e., rituximab); however, such reference is not intended to limit the present invention to a single anti-CD20 antibody. Indeed, all references to rituximab, or a biosimilar thereof, are to be read by a person skilled in the art to encompass the class of anti-CD20 antibodies. For example, it will be appreciated that the anti-CD20 antibodies ofatumumab (Arzerra®) or obinutuzumab (Gazyva®) can instead be administered in each instance where reference is made to rituximab. Thus, in some embodiments, provided methods comprise administering Compound 1, lenalidomide and/or and ofatumumab. In some such embodiments, ofatumumab is administered in 12 doses according to the following schedule: 300 mg initial dose, followed 1 week later by 2000 mg dose weekly for 7 doses, followed 4 weeks later by 2000 mg every 4 weeks for 4 doses. In some embodiments, provided methods comprise administering Compound 1, lenalidomide and/or and obinutuzumab. In some such embodiments, obinutuzumab is administered for six 28-day cycles as follows: 100 mg on day 1, cycle 1; 900 mg on day 2 cycle 1; 1000 mg on days 8 and 15 of cycle 1; and 1000 mg on day 1 of cycles 2-6. Accordingly, in some embodiments, the term “rituximab” encompasses all corresponding anti-CD20 antibodies that fulfill the requirements necessary for obtaining a marketing authorization as an identical or biosimilar product in a country or territory selected from the group of countries consisting of the USA, Europe and Japan.

In some embodiments, an anti-CD20 antibody has the same or similar activity as rituximab, or a biosimilar thereof. In some embodiments, an anti-CD20 antibody binds to the same or similar region or epitope as rituximab or a fragment thereof. In some embodiments, an anti-CD20 antibody competes with the binding of rituximab or a fragment thereof to CD20. In some embodiments, an anti-CD20 antibody is bioequivalent to rituximab or a fragment thereof. In some embodiments, an anti-CD20 antibody is a biosimilar of rituximab or a fragment thereof In some embodiments, an anti-CD20 antibody is a variant or derivative of rituximab, including functional fragments, derivatives, or antibody conjugates.

Rituximab

Rituximab (Rituxan® or MabThera®) is a genetically engineered cytolytic, chimeric murine/human monoclonal IgG₁ kappa antibody directed against the CD20 cell-surface molecule present in normal B lymphocytes and B-cell CLL and in most forms of non-Hodgkin's B-cell lymphomas. Rituximab has a binding affinity for the CD20 antigen of approximately 8.0 nM. Rituximab can induce complement-dependent cellular cytotoxicity (CDC) and anti-body-dependent cellular cytotoxicity (ADCC), leading to its clinical activity against lymphoma cells. Rituximab can also lead to apoptosis of B cells upon binding to CD20, thereby leading to direct inhibition of cellular growth.

Rituximab is produced by mammalian cell (Chinese Hamster Ovary) suspension culture in a nutrient medium containing the antibiotic gentamicin. Gentamicin is not detectable in the final product. Rituximab is a sterile, clear, colorless, preservative-free liquid concentrate for intravenous administration. Rituximab is supplied at a concentration of 10 mg/mL in either 100 mg/10 mL or 500 mg/50 mL single-use vials. Rituximab is formulated in polysorbate 80 (0.7 mg/mL), sodium citrate dihydrate (7.35 mg/mL), sodium chloride (9 mg/mL) and water for injection. The pH of Rituxan® (or MabThera®) is 6.5

Rituximab has been investigated in clinical studies and approved for treatment of patients with CLL along with fludarabine and cyclophosphamide, as well as patients with rheumatoid arthritis along with methotrexate. Rituximab is also approved for treatment of non-Hodgkin's lymphoma, Wegener's Granulomatosis and Microscopic Polyangiitis. Further, studies have shown that rituximab and bendamustine have also been effective for treatment of relapsed or refractory CLL and NHL, for example, indolent lymphoma and mantle-cell lymphoma. It will be understood and appreciated that when rituximab is administered along with Compound 1 and lenalidomide, rituximab can be used as a single agent or together with either (i) fludarabine and cyclophosphamide (“FCR”) or (ii) bendamustine (“BR”). Accordingly, in some embodiments, provided methods comprise administering each of Compound 1, lenalidomide, rituximab, fludarabine and cyclophosphamide. In some embodiments, provided methods comprise administering each of Compound 1, lenalidomide, rituximab and bendamustine.

### I. General Dosing Protocol

As described herein, provided methods comprise administering two or more therapeutic agents (i.e., Compound 1, lenalidomide and/or an anti-CD20 antibody). It will be appreciated that each of the therapeutic agents can be administered simultaneously or sequentially (e.g., Compound 1 can be administered before, during or after lenalidomide and/or an anti-CD20 antibody and vice versa) as part of a dosing regimen. For example, Compound 1 may be administered one or more hours, days or weeks before administration of either lenalidomide or an anti-CD20 antibody. In other embodiments, each of Compound 1 and lenalidomide may be administered one or more hours, days or weeks before administration of an anti-CD20 antibody. In still other embodiments, each of Compound 1 and an anti-CD20 antibody may be administered one or more hours, days or weeks before administration of lenalidomide.

In some embodiments, the present invention provides methods for treating, stabilizing or lessening the severity or progression of one or more diseases or conditions associated with BTK. In some embodiments, the present invention provides methods for preventing the progression of a disease or disorder associated with BTK. In some embodiments, the disease or disorder associated with BTK is selected from chronic lymphocytic leukemia (CLL) and small lymphocytic lymphoma (SLL).

In some embodiments, the disease or disorder associated with BTK is refractory CLL. In some embodiments, the disease or disorder associated with BTK is relapsed CLL. In some embodiments, the disease or disorder associated with BTK is refractory SLL. In some embodiments, the disease or disorder associated with BTK is relapsed SLL.

In some embodiments, provided methods comprise administering to a patient in need thereof Compound 1 in combination with lenalidomide. In some such embodiments, each of Compound 1 and lenalidomide is administered as a composition further comprising one or more pharmaceutically acceptable excipients. In some embodiments, provided methods further comprise administering an anti-CD20 antibody, e.g., rituximab. In some embodiments, provided methods comprise administering each of Compound 1, lenalidomide and an anti-CD20 antibody, e.g., rituximab.

In some embodiments, provided methods comprise administering to a patient in need thereof a therapeutically effective amount of Compound 1 in combination with a therapeutically effective amount of lenalidomide. Accordingly, in some embodiments, the present invention provides a method of treating, stabilizing or lessening the severity or progression of one or more diseases associated with BTK, the method comprising administering to a patient in need thereof a therapeutically effective amount of Compound 1 in combination with a therapeutically effective amount of lenalidomide. In some embodiments, provided methods further comprise administering an anti-CD20 antibody, e.g., rituximab. In some embodiments, provided methods comprise administering therapeutically effective amounts of each of Compound 1, lenalidomide and an anti-CD20 antibody, e.g., rituximab.

In some embodiments, provided methods comprise administering Compound 1 in combination with lenalidomide, wherein each of Compound 1 and lenalidomide is administered once daily (“QD”). In some embodiments, provided methods comprise administering Compound 1 in combination with lenalidomide, wherein Compound 1 is administered twice daily (“BID”). In some embodiments, provided methods further comprise administering an anti-CD20 antibody, e.g., rituximab. In some embodiments, provided methods comprise administering each of Compound 1, lenalidomide and an anti-CD20 antibody, e.g., rituximab, wherein Compound 1 is administered BID. In some such embodiments, lenalidomide is administered QD. For purposes of clarity, administration of a 375 mg dose of Compound 1 “BID” means that the patient is administered two separate doses of 375 mg in one day.

In some embodiments, provided methods comprise administering Compound 1 in combination with lenalidomide, wherein lenalidomide is administered once daily. In some embodiments, provided methods comprise administering Compound 1 in combination with lenalidomide, wherein Compound 1 is administered twice daily and lenalidomide is administered once daily. In some embodiments, provided methods further comprise administering an anti-CD20 antibody, e.g., rituximab, wherein the anti-CD20 antibody is administered once during a 28-day cycle. In some such embodiments, an anti-CD20 antibody is administered on day 1 or 2 of cycle 1. In some such embodiments, the anti-CD20 antibody is administered on day 1 of a 28-day cycle. In some embodiments, the anti-CD20 antibody is administered on day 1 of cycles 2-6. In some embodiments, provided methods comprise administering each of Compound 1, lenalidomide and an anti-CD20 antibody, wherein the anti-CD20 antibody is administered once during a 28-day cycle. In some such embodiments, the anti-CD20 antibody is administered on day 1 or day 2 of a 28-day cycle. It is understood that although the methods described herein refer to administering Compound 1, such methods are equally applicable to methods of administering a salt form of Compound 1, e.g., a besylate salt of Compound 1.

In some embodiments, each of Compound 1 and lenalidomide is administered as pharmaceutically acceptable compositions. In some such embodiments, each pharmaceutically acceptable composition is formulated as an oral dosage form. In some embodiments, such oral dosage forms are capsules. In some embodiments, provided methods further comprise administering a pharmaceutically acceptable composition of an anti-CD20 antibody, e.g., rituximab. In some embodiments, provided methods comprise administering pharmaceutically acceptable compositions of each of Compound 1, lenalidomide and an anti-CD20 antibody, e.g., rituximab. In some such embodiments, the pharmaceutically acceptable composition of an anti-CD20 antibody is formulated as an intravenous composition.

In some embodiments, a pharmaceutically acceptable composition comprising Compound 1 comprises from about 5% to about 60% of Compound 1, or a pharmaceutically acceptable salt thereof, based upon total weight of the composition. In some embodiments, a pharmaceutically acceptable composition comprising Compound 1 comprises from about 5% to about 15% or about 7% to about 15% or about 7% to about 10% or about 9% to about 12% of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of Compound 1, based upon total weight of the formulation. In certain embodiments, provided regimens comprise administering to a patient in need thereof a pharmaceutically acceptable composition comprising from about 6%, about 7%, about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of Compound 1, based upon total weight of given composition or formulation.

In some embodiments, a pharmaceutically acceptable composition comprising lenalidomide comprises from about 2.5% to about 6.25% of lenalidomide, based on the total weight of the composition. In some embodiments, a pharmaceutically acceptable composition comprises from about 2.5%, about 3.75% or about 6.25% of lenalidomide, or a pharmaceutically acceptable salt thereof, based upon total weight of the composition. In some embodiments, the composition of lenalidomide is the composition that is commercially available from Celgene Corporation. In some embodiments, a pharmaceutically acceptable composition of lenalidomide is the composition described in Table 2 or Table 3.

Rituximab is commercially available as a 10 mg/mL solution comprising sodium citrate, polysorbate 80, sodium chloride, sodium hydroxide, hydrochloric acid and water. Commercially available vials comprise either 100 mg/10 mL or 500 mg/50 mL.

In some embodiments, a pharmaceutically acceptable composition comprises from about 1 mg/mL to about 4 mg/mL rituximab. In some embodiments, a pharmaceutically acceptable composition comprises from about 1 mg/mL, about 2 mg/mL, about 3 mg/mL or about 4 mg/mL rituximab. In some embodiments, a pharmaceutically acceptable composition comprises 10 mg/mL.

In some embodiments, provided methods comprise administering Compound 1 in combination with lenalidomide daily for a period of 1, 2, 3, 4, 5, 6, 7, 8, 9, 10, 11, 12, 13, 14, 15, 16, 17, 18, 19, 20, 21, 22, 23, 24, 25, 26, 27 or 28 days. In some embodiments, a treatment regimen comprises at least one 28-day cycle. As used herein, the term “28-day cycle” means that the combination of Compound 1 and lenalidomide is administered to a patient in need thereof for 28 consecutive days. In some embodiments, the combination of Compound 1 and lenalidomide is administered once daily for at least one 28-day cycle. In some embodiments, the combination of Compound 1 and lenalidomide is administered for at least two, at least three, at least four, at least five or at least six 28-day cycles. In some embodiments, the combination of Compound 1 and lenalidomide is administered once daily for at least seven, at least eight, at least nine, at least ten, at least eleven or at least twelve 28-day cycles. In some embodiments, the combination of Compound 1 and lenalidomide is administered once daily for at least thirteen, at least fourteen, at least fifteen, at least sixteen, at least seventeen, at least eighteen, at least nineteen or at least twenty 28-day cycles. In some embodiments, the combination of Compound 1 and lenalidomide is administered to a patient for the duration of the patient's life. In some embodiments, Compound 1 is administered on days 1 through 28 (for example, one dose each day or two doses each day) of one or more 28-day cycles and lenalidomide is administered on days 1 through 21 (for example, one dose each day) of one or more 28-day cycles. In some embodiments, Compound 1 is administered on days 8 through 28 of one or more 28-day cycles and lenalidomide is administered on days 1 through 28 of one or more 28-day cycles. In some embodiments, Compound 1 is administered on days 1 through 28 of one or more 28-day cycles and lenalidomide is administered on days 1 through 28 of one or more 28-day cycles.

In some embodiments, each of Compound 1 and lenalidomide is administered to a patient for one, two or three 28-day cycles, whereafter the dose of lenalidomide is reduced. In some embodiments, each of Compound 1 and lenalidomide is administered to a patient for one, two or three 28-day cycles, whereafter the dose of lenalidomide is reduced to the previous cohort dose. In some embodiments, each of Compound 1 and lenalidomide is administered to a patient until a desired response is reached, e.g., a complete response, whereafter the dose of lenalidomide is reduced.

In some embodiments, each of Compound 1, lenalidomide and rituximab is administered for at least six 28-day cycles, and each of Compound 1 and lenalidomide is further administered for at least one additional 28-day cycle. In some embodiments, each of Compound 1, lenalidomide and rituximab is administered for at least six 28-day cycles, and each of Compound 1 and lenalidomide is further administered for an additional two, three, four, five, six, seven, eight, nine, ten, eleven, twelve, thirteen or fourteen 28-day cycles. In some embodiments, each of Compound 1, lenalidomide and rituximab is administered for at least six 28-day cycles, and each of Compound 1 and lenalidomide is further administered for the duration of the patient's life. In some embodiments, each of Compound 1 and lenalidomide is administered on days 1 through 28 (for example, one dose each day or two doses each day) of one or more 28-day cycles and rituximab is administered on day 1 of a 28-day cycle. In some embodiments, each of Compound 1 and lenalidomide is administered on days 1 through 28 of one or more 28-day cycles and rituximab is administered on day 1 or 2 of a 28-day cycle.

In some embodiments, each of Compound 1 and rituximab is administered to a patient during one or more cycles, whereafter each of Compound 1, rituximab and lenalidomide is administered to a patient during subsequent cycles. In some embodiments, each of Compound 1 and rituximab is administered to a patient during cycle 1, whereafter each of Compound 1, rituximab and lenalidomide is administered to a patient during cycles 2-6. In some embodiments, provided methods comprise (i) administering each of Compound 1 and rituximab to a patient in need thereof during a first 28-day cycle; (ii) administering each of Compound 1, rituximab and lenalidomide to the patient during cycles 2-6; and (iii) administering Compound 1 and/or lenalidomide to the patient during one or more 28-day cycles thereafter.

In some embodiments, two adjacent 28-day cycles may be separated by a rest period. Such a rest period may be one, two, three, four, five, six, seven or more days during which the patient is not administered either or both Compound 1 and lenalidomide. In a preferred embodiment, two adjacent 28-day cycles are continuous.

In some embodiments, provided methods comprise administering to a patient in need thereof Compound 1 in combination with lenalidomide, wherein the patient has failed at least one prior therapy. In some embodiments, provided methods comprise administering to a patient in need thereof each of Compound 1, lenalidomide and rituximab, wherein the patient has failed at least one prior therapy.

Unit Dosage Forms

Pharmaceutical compositions for use in the present invention may be prepared as a unit dosage form. A person of ordinary skill will appreciate that the unit dosage forms described herein refer to an amount of a component in its free base form. A person skilled in the art will further appreciate that, when a pharmaceutical composition comprises a salt form of one component, for example, a besylate salt form of Compound 1, the amount of the salt form present in the composition is an amount that is equivalent to a unit dose of the free base of the component (i.e., of Compound 1). For example, a pharmaceutical composition comprising a besylate salt of Compound 1 would contain 34.97 mg of the besylate salt form necessary to deliver an equivalent 25 mg unit dose of the free base of Compound 1.

In some embodiments, provided methods comprise administering to a patient in need thereof a therapeutically effective amount of Compound 1, wherein the therapeutically effective amount of Compound 1 is about 250 mg to about 1250 mg. In some embodiments, the therapeutically effective amount of Compound 1 is administered as one or more discreet doses. For example, in some embodiments, a therapeutically effective amount of Compound 1 is 250 mg, wherein the therapeutically effective amount is administered as 125 mg twice daily (BID). In some embodiments, a therapeutically effective amount of Compound 1 is 500 mg, wherein the therapeutically effective amount is administered as 250 mg twice daily (BID). In some embodiments, a therapeutically effective amount of Compound 1 is 750 mg, wherein the therapeutically effective amount is administered as 375 mg twice daily (BID). In some embodiments, a therapeutically effective amount of Compound 1 is 1000 mg, wherein the therapeutically effective amount is administered as 500 mg twice daily (BID).

In some embodiments, provided methods comprise administering to a patient in need thereof a therapeutically effective amount of Compound 1, wherein the therapeutically effective amount of Compound 1 is about 125 mg to about 1250 mg, or about 125 mg to about 1125 mg, or about 125 mg to about 1000 mg, or about 125 mg to about 875 mg, or about 125 mg to about 750 mg, or about 125 mg to about 625 mg, or about 125 mg to about 500 mg, or about 125 mg to about 375 mg, or about 125 mg to about 250 mg, or about 250 mg to about 1250 mg, or about 250 mg to about 1125 mg, or about 250 mg to about 1000 mg, or about 250 mg to about 875 mg, or about 250 mg to about 750 mg, or about 250 mg to about 625 mg, or about 250 mg to about 500 mg, or about 250 mg to about 375 mg, or about 375 mg to about 1250 mg, or about 375 mg to about 1125 mg, or about 375 mg to about 1000 mg, or about 375 mg to about 875 mg, or about 375 mg to about 750 mg, or about 375 mg to about 625 mg, or about 375 mg to about 500 mg, or about 500 mg to about 1250 mg, or about 500 mg to about 1125 mg, or about 500 mg to about 1000 mg, or about 500 mg to about 875 mg, or about 500 mg to about 750 mg, or about 500 mg to about 625 mg, or about 625 mg to about 1250 mg, or about 625 mg to about 1125 mg, or about 625 mg to about 1000 mg, or about 625 mg to about 875 mg, or about 625 mg to about 750 mg, or about 750 mg to about 1250 mg, or about 750 mg to about 1125 mg, or about 750 mg to about 1000 mg, or about 875 mg to about 1250 mg, or about 875 mg to about 1125 mg, or about 875 mg to about 1000 mg.

In some embodiments, provided methods comprise administering to a patient in need thereof a therapeutically effective amount of Compound 1, wherein the therapeutically effective amount of Compound 1 is about 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, 1005 mg, 1010 mg, 1015 mg, 1020 mg, 1025 mg, 1030 mg, 1035 mg, 1040 mg, 1045 mg, 1050 mg, 1055 mg, 1060 mg, 1065 mg, 1070 mg, 1075 mg, 1080 mg, 1085 mg, 1090 mg, 1095 mg, 1100 mg, 1105 mg, 1110 mg, 1115 mg, 1120 mg, 1125 mg, 1130 mg, 1135 mg, 1140 mg, 1145 mg, 1150 mg, 1155 mg, 1160 mg, 1165 mg, 1170 mg, 1175 mg, 1180 mg, 1185 mg, 1190 mg, 1195 mg, 1200 mg, 1205 mg, 1210 mg, 1215 mg, 1220 mg, 1225 mg, 1230 mg, 1235 mg, 1240 mg, 1245 mg or 1250 mg.

In some embodiments, provided methods comprise administering to a patient in need thereof a therapeutically effective amount of lenalidomide, wherein the therapeutically effective amount of lenalidomide is about 2.5 mg to about 25 mg.

In some embodiments, provided methods comprise administering to a patient in need thereof a therapeutically effective amount of lenalidomide, wherein the therapeutically effective amount of lenalidomide is about 2.5 mg to about 25 mg, or about 2.5 mg to about 20 mg, or about 2.5 mg to about 15 mg, or about 2.5 mg to about 10 mg, or about 2.5 mg to about 7.5 mg, or about 2.5 mg to about 5 mg, or about 5 mg to about 25 mg, or about 5 mg to about 20 mg, or about 5 mg to about 15 mg, or about 5 mg to about 10 mg, or about 5 mg to about 7.5 mg, or about 7.5 mg to about 25 mg, or about 7.5 mg to about 20 mg, or about 7.5 mg to about 15 mg, or about 7.5 mg to about 10 mg, or about 10 mg to about 25 mg, or about 10 mg to about 20 mg, or about 10 mg to about 15 mg, or about 15 mg to about 25 mg, or about 15 mg to about 20 mg, or about 20 mg to about 25 mg.

In some embodiments, provided methods comprise administering to a patient in need thereof about 2.5 mg, 5 mg, about 7.5 mg, 10 mg, 15 mg, 20 mg, 25 mg of lenalidomide.

In some embodiments, provided methods comprise administering to a patient in need thereof a pharmaceutical composition comprising a unit dose of Compound 1 in combination with a unit dose of lenalidomide. In some such embodiments, the unit dose of Compound 1 is about 25 mg, about 50 mg, about 75 mg, about 100 mg, about 125 mg, about 150 mg, about 175 mg, about 200 mg, about 225 mg or about 250 mg.

In some embodiments, the unit dose of lenalidomide is about 2.5 mg, about 5 mg, about 7.5 mg, about 10 mg, about 15 mg or about 25 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

### II. Uses of Compounds and Pharmaceutically Acceptable Compositions

Compound 1 and compositions described herein are generally useful for the inhibition of protein kinase activity of one or more enzymes. Examples of kinases that are inhibited by Compound 1 and compositions described herein and against which the methods described herein are useful include BTK and other TEC-kinases, including ITK, TEC, BMX and RLK, or a mutant thereof.

Bruton's tyrosine kinase (“BTK”), a member of TEC-kinases, is a key signaling enzyme expressed in B-lymphocytes, monocytes, and mast cells or basophils. BTK plays an essential role in the B-cell signaling pathway linking cell surface B-cell receptor (BCR) stimulation to downstream intracellular responses.

BTK is a key regulator of B-cell development, activation, signaling, and survival (Kurosaki, Curr. Op. Imm., 2000, 276-281; Schaeffer and Schwartzberg, Curr. Op. Imm. 2000, 282-288). In addition, BTK plays a role in a number of other hematopoietic cell signaling pathways, e.g., Toll like receptor (TLR) and cytokine receptor-mediated TNF-α production in macrophages, IgE receptor (Fc_epsilon_RI) signaling in mast cells, inhibition of Fas/APO-1 apoptotic signaling in B-lineage lymphoid cells, and collagen-stimulated platelet aggregation. See, e.g., C. A. Jeffries, et al., (2003), Journal of Biological Chemistry 278:26258-26264; N. J. Horwood, et al., (2003), The Journal of Experimental Medicine 197: 1603-1611; Iwaki et al. (2005), Journal of Biological Chemistry 280(48):40261-40270; Vassilev et al. (1999), Journal of Biological Chemistry 274(3): 1646-1656, and Quek et al. (1998), Current Biology 8(20): 1137-1140.

Patients with inherited inactivating mutations in BTK have a profound block in B-cell development, resulting in the almost complete absence of mature B lymphocytes and plasma cells, severely reduced Ig levels and a profound inhibition of humoral response to recall antigens (reviewed in Vihinen et al Frontiers in Bioscience 5: d917-928). Mice deficient in BTK also have a reduced number of peripheral B-cells and greatly decreased serum levels of IgM and IgG3. BTK deletion in mice has a profound effect on B-cell proliferation induced by anti-IgM, and inhibits immune responses to thymus-independent type II antigens (Ellmeier et al, J Exp Med 192: 1611-1623 (2000)). BTK also plays a crucial role in mast cell activation through the high-affinity IgE receptor (Fc_epsilon_RI). BTK deficient murine mast cells have reduced degranulation and decreased production of proinflammatory cytokines following Fc_epsilon_RI cross-linking (Kawakami et al. Journal of Leukocyte Biology 65: 286-290).

Compound 1 is an inhibitor of BTK and therefore useful for treating one or more disorders associated with activity of BTK. Thus, in some embodiments, the present invention provides a method of treating, stabilizing or lessening the severity or progression of a BTK-mediated disorder comprising the step of administering to a patient in need thereof Compound 1 in combination with lenalidomide.

Chronic Lymphocytic Leukemia and Small Lymphocytic Lymphoma

The B-cell disorders chronic lymphocytic leukemia (CLL) and small lymphocytic lymphoma (SLL) represent two ends of a spectrum of the same disease process differing in the degree of blood/marrow involvement (CLL) versus lymph node involvement (SLL). CLL is a lymphoproliferative malignancy characterized by progressive accumulation of morphologically mature but functionally incompetent lymphocytes in the blood, bone marrow, and lymphoid tissues. It affects mainly elderly individuals with the median age at presentation of 65 to 70 years. The clinical course of CLL ranges from indolent disease with long-term survival over 12 years to aggressive disease with median survival of 2 years.

Chronic lymphocytic leukemia is the most common leukemia in the U.S. and is typically characterized immunophenotypically as CD5+, CD23+, CD10−, CD19+, CD20 dim, sIg dim, and cyclin D1− (the latter point a distinguishing feature from mantle cell lymphoma). Chronic lymphocytic leukemia must also be distinguished from monoclonal B lymphocytosis (absolute monoclonal B-cell count<5000/μL and absence of adenopathy or other clinical features of lymphoproliferative disorder). The understanding of CLL/SLL biology and prognostic factors, and advances in formulating a risk-stratified approach to treatment of CLL/SLL have been recently reviewed by Lanasa, Furman, and the National Comprehensive Cancer Network NHL panel.

The cellular expression of Btk is restricted and largely limited to B-lymphocytes, monocytes, and mast cells or basophils. Investigation has revealed that some B-cell lymphomas and CLL/SLL depend on BCR signaling, suggesting that interruption of such signaling could be a promising therapeutic opportunity Recently it has been reported that half of all CLL retain BCR signaling in vitro and that immunoglobulin heavy gene somatic mutation (IgVH) is an important determinant of BCR responsiveness. Indeed, the mutational status of the BCR in CLL is one of the strongest predictors of disease progression, as aggressive disease typically displays BCR encoded by unmutated immunoglobulin variable heavy chains.

Two groups have reported that mutated and unmutated CLL cells respond differentially to IgM ligation of the BCR, with unmutated, but not mutated, CLL cells responding to BCR stimulation with increased global tyrosine phosphorylation and by up-regulating several genes associated with cell cycle regulation and allowing cell growth and expansion. These data highlight the differential role that BCR signaling plays in CLL physiology depending on IgVH mutational status and may suggest a possible differential responsiveness of CLL to inhibitors of BCR signaling. Other in vitro studies have reported that specific Btk inhibition with the investigational agent PCI-32765 produced substantially more apoptosis and cytotoxicity in CLL cells relative to normal B-cells; as well as inducing apoptosis in the face of anti-apoptotic micro-environmental signals, reduction of secretion of chemokines CCL3 and CCL4, and reduction of chemotaxis towards the chemokines CXCL12 and CXCL13. Detailed studies of the pathophysiologic role of Btk in the origin and/or maintenance of WM have not yet been reported. However, a recent report investigating transgenic mouse models demonstrated that constitutively active Btk expression resulted in selective expansion or survival of B-1 cells that were driven into germinal center independent plasma cell differentiation, as evidenced by increased numbers of IgM+ plasma cells in spleen and bone marrow and significantly elevated serum IgM. Anti-nucleosome autoantibodies and glomerular IgM deposition were also observed. However, one study of sequence analysis in 19 WM patients with hypogammaglobulinemia G and/or A failed to find any novel variants in the promoter, flanking introns, or exons of Btk.

Allogeneic stem cell transplant is the only potentially curative treatment for CLL, but 70% of affected patients are ≧65 years of age at the time of diagnosis, have co-morbid conditions limiting eligibility for such therapy, and may exhibit a prolonged natural history with or without specific treatment. The actual prognosis of CLL is variable and dependent principally on clinical stage and certain genetic and molecular features. Both the Rai and Binet clinical staging systems are able to distinguish patient prognostic groups with median OSs ranging from 19 months in the most advanced stage (thrombocytopenia) to >150 months in the earliest stage (blood and marrow lymphocytosis without adenopathy, organomegaly, or defined anemia/thrombocytopenia). Classification by the presence or absence of IgVH and by interphase fluorescent in situ hybridization (iFISH) analysis for probed-for acquired chromosomal abnormalities adds additional prognostic discrimination to clinical staging, with unmutated IgVH and del(11q) and del(17p) cytogenetics predicting poorer outcome.

The CLL treatment algorithm is complex and requires first the decision to treat (e.g., presence of symptoms such as fatigue or night sweats; bulky adenopathy/organomegaly; progressive anemia/thrombocytopenia); and second, choice of the treatment regimen, usually involving one or more: purine nucleosides (fludarabine), alkylating agents (cyclophosphamide, chlorambucil, bendamustine), corticosteroids, anti-CD20 monoclonal antibodies (rituximab/ofatumumab), or anti-CD52 monoclonal antibodies (alemtuzumab). The choice of specific therapies depends on the patient's age, disease pattern (eg, primarily nodal versus non-nodal), anticipated drug tolerance and contraindications, and presence or absence of adverse prognostic features such as del(11q) or del(17p). Despite numerous therapies, treatment options are eventually limited by drug toxicities and resistance, and patients who do not succumb to other maladies endure progressive complications relating to cytopenias, the effects of lymphadenopathy and organomegaly, systemic symptoms, and infectious complications. Given the often elderly character of the patient population, an orally available, well tolerated treatment that exploits a novel weakness of CLL would be welcome.

Rationale for Targeting Btk and Combinations with Lenalidomide in CLL and SLL

Strategies specifically targeting B-cells, for example the B-cell depleting anti-CD20 monoclonal antibodies rituximab and ofatumumab, have demonstrated clinical efficacy in B-cell lymphoma and CLL. Spleen tyrosine kinase (Syk) is a kinase in the BCR signaling pathway proximal to Btk. Inhibition of Syk with the orally available Syk inhibitor fostamatinib disodium produced clinical responses in DLBCL, CLL, and mantle cell lymphoma. Most tellingly, clinical proof of concept for Btk inhibition has been demonstrated by clinical investigations of the orally available Btk inhibitor PCI-32765, which have reported objective anti-tumor responses in patients with DLBCL; mantle cell, marginal zone/mucosa-associated lymphoid tissue (MALT), and follicular lymphoma (FL), Waldenstrom's macroglobulinemia (WM), and CLL/SLL, with good tolerability.

Thus, based on the critical importance of BCR signaling mediated through Btk for the survival and proliferation of various malignant B-cells; Btk's limited cellular expression in B-cells, macrophages, and monocytes; and demonstrated pre-clinical and early clinical proofs of concept that Btk inhibition produces salutary anti-lymphoma, CLL, and WM effects with acceptable clinical tolerability, targeting Btk with a selective Btk inhibitor is a promising and appropriate therapeutic strategy to investigate further in the clinic. Compound 1, as its besylate salt, has been shown in recent studies to be safe and effective against CLL as a single agent therapeutic. As of Sep. 11, 2012, 35 out of 43 patients with CLL have experienced stable disease and continue to on treatment with N-(3-(5-fluoro-2-(4-(2-methoxyethoxyl)phenylamino)pyrimidin-4-ylamino)phenyl)acrylamide besylate monotherapy. 15 of 23 patients have experienced reductions in lymph node size, and 28 of 33 patients have experienced early increases in absolute lymphocyte counts (ALC). See U.S. patent application Ser. No. 13/661,678 and International Patent Application No. PCT/US2012/062133, both filed on Oct. 26, 2012, each of which is hereby incorporated by reference in its entirety. Such data strongly support the use of a BTK inhibitor, and Compound 1 in particular, for treating CLL. Compound 1 is generally well tolerated as a single agent at up to 750 mg PO QD and the maximum tolerated dose (MTD) has not yet been reached. Studies are ongoing and additional dose levels currently being investigated include: 1000 mg QD, 1250 mg QD, 375 mg BID and 500 mg BID. Lenalidomide has also been shown to exhibit good activity against relapsed/refractory CLL patients.

Rituximab has also been shown to exhibit good activity against relapsed/refractory CLL patients. In one study, rituximab, fludarabine and cyclophosphamide was evaluated in 408 patients with CLL and showed an 86% response rate, as compared to the 73% response rate observed for fludarabine/cyclophosphamide alone. The median progression-free survival was 39.8 months, as compared to 31.5 months observed for fludarabine/cyclophosphamide alone.

Accordingly, in some embodiments, the present invention encompasses the recognition that a BTK inhibitor such as Compound 1 in combination with lenalidomide is useful in the treatment of CLL and SLL. In some embodiments, the present invention encompasses the recognition that a BTK inhibitor such as Compound 1, together with lenalidomide and rituximab, is useful in the treatment of CLL and SLL. Compound 1 either as a single agent or in combination, may be found to be efficacious in CLL patients, including but not limited to those who had expressed one or more of the following prognostic/genetic markers and cytogenetic risk factors: deletions of chromosome 11q, 17p or 13q, or Trisomy 12 and 14q, zeta-chain-associated protein kinase 70 (ZAP 70) or immunoglobulin heavy chain variable region (IgVH) un-mutated.

In some embodiments, the present invention encompasses the recognition that lenalidomide and Btk inhibition are synergistic in diffuse large B-cell lymphoma (DLBCL) models, perhaps through concomitant inhibition of IkappaB kinase (IKK) and downstream nuclear factor kappa B (NFkB) signaling by both agents. Accordingly, the present invention provides methods of treating, stabilizing or lessening the severity or progression of one or more diseases and conditions associated with BTK comprising administering to a patient in need thereof Compound 1 in combination with lenalidomide. In some embodiments, provided methods further comprise administering to a patient in need thereof rituximab. In some embodiments, provided methods comprise administering to a patient in need thereof each of Compound 1, lenalidomide and rituximab.

III. Methods of Treating Diseases or Disorders Associated with BTK

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with lenalidomide. In some embodiments, provided methods further comprise administering to a patient in need thereof rituximab. In some embodiments, provided methods comprise administering to a patient in need thereof each of Compound 1, lenalidomide and rituximab.

In some embodiments, the present invention provides a combination of Compound 1 and lenalidomide, wherein the combination demonstrates synergistic effects. In some embodiments, provided methods comprise administering to a patient in need thereof Compound 1 in combination with lenalidomide, wherein the amount(s) of Compound 1 and/or lenalidomide administered are less than the amount administered for one or both compounds when dosed in separate therapeutic regimens. In some embodiments, provided methods further comprise administering to a patient in need thereof rituximab. In some embodiments, provided methods comprise administering to a patient in need thereof each of the therapeutic agents Compound 1, lenalidomide and rituximab wherein such therapeutic agents demonstrate synergistic effects.

In some embodiments, provided methods comprise administering to a patient in need thereof a composition comprising Compound 1 in combination with a composition comprising lenalidomide. In some embodiments, provided methods further comprise administering to a patient in need thereof a composition comprising rituximab. In some embodiments, provided methods comprise administering to a patient in need thereof compositions comprising each of Compound 1, lenalidomide and rituximab.

In some embodiments, the composition comprising Compound 1 further comprises one or more pharmaceutically acceptable excipients. In some such embodiments, the composition comprising Compound 1 is formulated as an oral dosage form. In some embodiments, the oral dosage form is a capsule.

In some embodiments, the composition comprising lenalidomide further comprises one or more pharmaceutically acceptable excipients. In some such embodiments, the composition comprising lenalidomide is formulated as an oral dosage form. In some embodiments, the oral dosage form is a capsule.

In some embodiments, the combination of Compound 1 and lenalidomide is administered as a single pharmaceutically acceptable composition.

In some embodiments, provided methods comprise administering to a patient in need thereof a unit dose of Compound 1 in combination with a unit dose of lenalidomide. In some embodiments, provided methods further comprise administering to a patient in need thereof a unit dose of rituximab. In some embodiments, provided methods comprise administering to a patient in need thereof a unit dose of Compound 1, a unit dose of lenalidomide and a unit dose of rituximab. In some embodiments, provided methods further comprise administering to a patient in need thereof a unit dose of rituximab. In some embodiments, the unit dose of Compound 1 is about 25 mg, about 50 mg, about 75 mg, about 100 mg, about 125 mg, about 150 mg, about 175 mg, about 200 mg, about 225 mg or about 250 mg. In some embodiments, a unit dose of lenalidomide is about 2.5 mg, about 5 mg, about 7.5 mg, about 10 mg, about 15 mg or about 25 mg.

In some embodiments, provided methods comprise administering to a patient in need thereof a pharmaceutical composition comprising rituximab, wherein rituximab is administered as an infusion at a rate of 50 mg/hr. In some embodiments, the infusion rate of rituximab is increased by 50 mg/hr every 30 minutes, to a maximum of 400 mg/hr. In some embodiments, the infusion rate of rituximab is increased by 100 mg/hr every 30 minutes, to a maximum of 400 mg/hr. Accordingly, in some embodiments, the infusion rate of rituximab is 100 mg/hr. In some embodiments, the infusion rate of rituximab is 150 mg/hr. In some embodiments, the infusion rate of rituximab is 200 mg/hr. In some embodiments, the infusion rate of rituximab is 250 mg/hr. In some embodiments, the infusion rate of rituximab is 300 mg/hr. In some embodiments, the infusion rate of rituximab is 350 mg/hr. In some embodiments, the infusion rate of rituximab is 400 mg/hr.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with lenalidomide, wherein the patient has failed at least one prior therapy. In some embodiments, provided methods comprise administering to a patient in need thereof each of Compound 1, lenalidomide and rituximab, wherein the patient has failed at least one prior therapy.

In some embodiments, provided methods comprise administering to a patient in need thereof about 250 mg to about 1250 mg Compound 1 in combination with about 2.5 mg to about 25 mg lenalidomide. In some embodiments, provided methods comprise administering to a patient in need thereof about 500 mg to about 1250 mg Compound 1 in combination with about 2.5 mg to about 25 mg lenalidomide. In some embodiments, provided methods comprise administering to a patient in need thereof about 250 mg to about 1250 mg Compound 1, about 2.5 mg to about 25 mg lenalidomide and about 375 mg/m² to about 500 mg/m² rituximab.

In some embodiments, provided methods comprise administering to a patient in need thereof about 750 mg to about 1000 mg Compound 1 and about 2.5 mg to about 10 mg lenalidomide. In some embodiments, provided methods comprise administering to a patient in need thereof about 750 mg to about 1000 mg Compound 1 and about 2.5 mg to about 15 mg lenalidomide. In some embodiments, provided methods comprise administering to a patient in need thereof about 750 mg to about 1000 mg Compound 1 and about 5 mg to about 10 mg lenalidomide. In some embodiments, provided methods comprise administering to a patient in need thereof about 750 mg to about 1000 mg Compound 1 and about 5 mg to about 15 mg lenalidomide. In some embodiments, provided methods comprise administering to a patient in need thereof about 750 mg to about 1000 mg Compound 1 and about 7.5 mg to about 10 mg lenalidomide. In some embodiments, provided methods comprise administering to a patient in need thereof about 750 mg to about 1000 mg Compound 1 and about 7.5 mg to about 15 mg lenalidomide. In some embodiments, provided methods comprise administering to a patient in need thereof about 750 mg to about 1000 mg Compound 1 and about 10 mg to about 15 mg lenalidomide. In some embodiments, provided methods further comprise administering to a patient in need thereof about 375 mg/m² to about 500 mg/m² rituximab.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof about 125 mg BID to about 500 mg BID Compound 1 in combination with about 2.5 mg QD to about 15 mg QD lenalidomide. In some embodiments, provided methods further comprise administering to a patient in need thereof about 375 mg/m² to about 500 mg/m² rituximab. In some embodiments, provided methods comprise administering to a patient in need thereof about 125 mg BID to about 500 mg BID Compound 1, about 2.5 mg QD to about 15 mg QD lenalidomide and about 375 mg/m² to about 500 mg/m² rituximab.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof about 125 mg BID to about 500 mg BID Compound 1 in combination with about 5 mg QD to about 15 mg QD lenalidomide. In some embodiments, provided methods further comprise administering to a patient in need thereof about 375 mg/m² to about 500 mg/m² rituximab. In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof about 125 mg BID to about 500 mg BID Compound 1, about 2.5 mg QD to about 15 mg QD lenalidomide and about 375 mg/m² to about 500 mg/m² rituximab.

In some embodiments, provided methods comprise administering to a patient in need thereof about 125 mg BID to about 500 mg BID Compound 1 and about 2.5 mg QD to about 15 mg QD lenalidomide. In some embodiments, provided methods comprise administering to a patient in need thereof about 125 mg BID to about 500 mg BID Compound 1 and about 2.5 mg QD to about 10 mg QD lenalidomide. In some embodiments, provided methods comprise administering to a patient in need thereof about 125 mg BID to about 500 mg BID Compound 1 and about 2.5 mg QD to about 7.5 mg QD lenalidomide. In some embodiments, provided methods comprise administering to a patient in need thereof about 125 mg BID to about 500 mg BID Compound 1 and about 2.5 mg QD to about 5 mg QD lenalidomide. In some embodiments, provided methods comprise administering to a patient in need thereof about 125 mg BID to about 500 mg BID Compound 1 and about 5 mg QD to about 15 mg QD lenalidomide. In some embodiments, provided methods comprise administering to a patient in need thereof about 125 mg BID to about 500 mg BID Compound 1 and about 5 mg QD to about 10 mg QD lenalidomide. In some embodiments, provided methods comprise administering to a patient in need thereof about 125 mg BID to about 500 mg BID Compound 1 and about 5 mg QD to about 7.5 mg QD lenalidomide. In some embodiments, provided methods comprise administering to a patient in need thereof about 125 mg BID to about 500 mg BID Compound 1 and about 7.5 mg QD to about 15 mg QD lenalidomide. In some embodiments, provided methods comprise administering to a patient in need thereof about 125 mg BID to about 500 mg BID Compound 1 and about 7.5 mg QD to about 10 mg QD lenalidomide. In some embodiments, provided methods comprise administering to a patient in need thereof about 125 mg BID to about 500 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID to about 375 mg BID Compound 1 and about 2.5 mg QD to about 15 mg QD lenalidomide. In some embodiments, provided methods comprise administering to a patient in need thereof about 125 mg BID to about 375 mg BID Compound 1 and about 2.5 mg QD to about 10 mg QD lenalidomide. In some embodiments, provided methods comprise administering to a patient in need thereof about 125 mg BID to about 375 mg BID Compound 1 and about 2.5 mg QD to about 7.5 mg QD lenalidomide. In some embodiments, provided methods comprise administering to a patient in need thereof about 125 mg BID to about 375 mg BID Compound 1 and about 2.5 mg QD to about 5 mg QD lenalidomide. In some embodiments, provided methods comprise administering to a patient in need thereof about 125 mg BID to about 375 mg BID Compound 1 and about 5 mg QD to about 15 mg QD lenalidomide. In some embodiments, provided methods comprise administering to a patient in need thereof about 125 mg BID to about 375 mg BID Compound 1 and about 5 mg QD to about 10 mg QD lenalidomide. In some embodiments, provided methods comprise administering to a patient in need thereof about 125 mg BID to about 375 mg BID Compound 1 and about 5 mg QD to about 7.5 mg QD lenalidomide. In some embodiments, provided methods comprise administering to a patient in need thereof about 125 mg BID to about 375 mg BID Compound 1 and about 7.5 mg QD to about 15 mg QD lenalidomide. In some embodiments, provided methods comprise administering to a patient in need thereof about 125 mg BID to about 375 mg BID Compound 1 and about 7.5 mg QD to about 10 mg QD lenalidomide. In some embodiments, provided methods comprise administering to a patient in need thereof about 125 mg BID to about 375 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID to about 250 mg BID Compound 1 and about 2.5 mg QD to about 15 mg QD lenalidomide. In some embodiments, provided methods comprise administering to a patient in need thereof about 125 mg BID to about 250 mg BID Compound 1 and about 2.5 mg QD to about 10 mg QD lenalidomide. In some embodiments, provided methods comprise administering to a patient in need thereof about 125 mg BID to about 250 mg BID Compound 1 and about 2.5 mg QD to about 7.5 mg QD lenalidomide. In some embodiments, provided methods comprise administering to a patient in need thereof about 125 mg BID to about 250 mg BID Compound 1 and about 2.5 mg QD to about 5 mg QD lenalidomide. In some embodiments, provided methods comprise administering to a patient in need thereof about 125 mg BID to about 250 mg BID Compound 1 and about 5 mg QD to about 15 mg QD lenalidomide. In some embodiments, provided methods comprise administering to a patient in need thereof about 125 mg BID to about 250 mg BID Compound 1 and about 5 mg QD to about 10 mg QD lenalidomide. In some embodiments, provided methods comprise administering to a patient in need thereof about 125 mg BID to about 250 mg BID Compound 1 and about 5 mg QD to about 7.5 mg QD lenalidomide. In some embodiments, provided methods comprise administering to a patient in need thereof about 125 mg BID to about 250 mg BID Compound 1 and about 7.5 mg QD to about 15 mg QD lenalidomide. In some embodiments, provided methods comprise administering to a patient in need thereof about 125 mg BID to about 250 mg BID Compound 1 and about 7.5 mg QD to about 10 mg QD lenalidomide. In some embodiments, provided methods comprise administering to a patient in need thereof about 125 mg BID to about 250 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID to about 500 mg BID Compound 1 and about 2.5 mg QD to about 15 mg QD lenalidomide. In some embodiments, provided methods comprise administering to a patient in need thereof about 250 mg BID to about 500 mg BID Compound 1 and about 2.5 mg QD to about 10 mg QD lenalidomide. In some embodiments, provided methods comprise administering to a patient in need thereof about 250 mg BID to about 500 mg BID Compound 1 and about 2.5 mg QD to about 7.5 mg QD lenalidomide. In some embodiments, provided methods comprise administering to a patient in need thereof about 250 mg BID to about 500 mg BID Compound 1 and about 2.5 mg QD to about 5 mg QD lenalidomide. In some embodiments, provided methods comprise administering to a patient in need thereof about 250 mg BID to about 500 mg BID Compound 1 and about 5 mg QD to about 15 mg QD lenalidomide. In some embodiments, provided methods comprise administering to a patient in need thereof about 250 mg BID to about 500 mg BID Compound 1 and about 5 mg QD to about 10 mg QD lenalidomide. In some embodiments, provided methods comprise administering to a patient in need thereof about 250 mg BID to about 500 mg BID Compound 1 and about 5 mg QD to about 7.5 mg QD lenalidomide. In some embodiments, provided methods comprise administering to a patient in need thereof about 250 mg BID to about 500 mg BID Compound 1 and about 7.5 mg QD to about 15 mg QD lenalidomide. In some embodiments, provided methods comprise administering to a patient in need thereof about 250 mg BID to about 500 mg BID Compound 1 and about 7.5 mg QD to about 10 mg QD lenalidomide. In some embodiments, provided methods comprise administering to a patient in need thereof about 250 mg BID to about 500 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 250 mg BID to about 375 mg BID Compound 1 and about 2.5 mg QD to about 15 mg QD lenalidomide. In some embodiments, provided methods comprise administering to a patient in need thereof about 250 mg BID to about 375 mg BID Compound 1 and about 2.5 mg QD to about 10 mg QD lenalidomide. In some embodiments, provided methods comprise administering to a patient in need thereof about 250 mg BID to about 375 mg BID Compound 1 and about 2.5 mg QD to about 7.5 mg QD lenalidomide. In some embodiments, provided methods comprise administering to a patient in need thereof about 250 mg BID to about 375 mg BID Compound 1 and about 2.5 mg QD to about 5 mg QD lenalidomide. In some embodiments, provided methods comprise administering to a patient in need thereof about 250 mg BID to about 375 mg BID Compound 1 and about 5 mg QD to about 15 mg QD lenalidomide. In some embodiments, provided methods comprise administering to a patient in need thereof about 250 mg BID to about 375 mg BID Compound 1 and about 5 mg QD to about 10 mg QD lenalidomide. In some embodiments, provided methods comprise administering to a patient in need thereof about 250 mg BID to about 375 mg BID Compound 1 and about 5 mg QD to about 7.5 mg QD lenalidomide. In some embodiments, provided methods comprise administering to a patient in need thereof about 250 mg BID to about 375 mg BID Compound 1 and about 7.5 mg QD to about 15 mg QD lenalidomide. In some embodiments, provided methods comprise administering to a patient in need thereof about 250 mg BID to about 375 mg BID Compound 1 and about 7.5 mg QD to about 10 mg QD lenalidomide. In some embodiments, provided methods comprise administering to a patient in need thereof about 250 mg BID to about 375 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 375 mg BID to about 500 mg BID Compound 1 and about 2.5 mg QD to about 15 mg QD lenalidomide. In some embodiments, provided methods comprise administering to a patient in need thereof about 375 mg BID to about 500 mg BID Compound 1 and about 2.5 mg QD to about 10 mg QD lenalidomide. In some embodiments, provided methods comprise administering to a patient in need thereof about 375 mg BID to about 500 mg BID Compound 1 and about 2.5 mg QD to about 7.5 mg QD lenalidomide. In some embodiments, provided methods comprise administering to a patient in need thereof about 375 mg BID to about 500 mg BID Compound 1 and about 2.5 mg QD to about 5 mg QD lenalidomide. In some embodiments, provided methods comprise administering to a patient in need thereof about 375 mg BID to about 500 mg BID Compound 1 and about 5 mg QD to about 15 mg QD lenalidomide. In some embodiments, provided methods comprise administering to a patient in need thereof about 375 mg BID to about 500 mg Compound 1 and about 5 mg QD to about 10 mg QD lenalidomide. In some embodiments, provided methods comprise administering to a patient in need thereof about 375 mg BID to about 500 mg Compound 1 and about 5 mg QD to about 7.5 mg QD lenalidomide. In some embodiments, provided methods comprise administering to a patient in need thereof about 375 mg BID to about 500 mg BID Compound 1 and about 7.5 mg QD to about 15 mg QD lenalidomide. In some embodiments, provided methods comprise administering to a patient in need thereof about 375 mg BID to about 500 mg BID Compound 1 and about 7.5 mg QD to about 10 mg QD lenalidomide. In some embodiments, provided methods comprise administering to a patient in need thereof about 375 mg BID to about 500 mg BID Compound 1 and about 10 mg QD to about 15 mg QD lenalidomide. In some embodiments, provided methods further comprise administering to a patient in need thereof about 375 mg/m² to about 500 mg/m² rituximab.

In some embodiments, provided methods comprise administering to a patient in need thereof about 125 mg BID Compound 1 and about 2.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 125 mg BID Compound 1 and about 5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 125 mg BID Compound 1 and about 7.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 125 mg BID Compound 1 and about 10 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 125 mg BID Compound 1 and about 15 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 250 mg BID Compound 1 and about 2.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 250 mg BID Compound 1 and about 5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 250 mg BID Compound 1 and about 7.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 250 mg BID Compound 1 and about 10 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 250 mg BID Compound 1 and about 15 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 375 mg BID Compound 1 and about 2.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 375 mg BID Compound 1 and about 5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 375 mg BID Compound 1 and about 7.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 375 mg BID Compound 1 and about 10 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 375 mg BID Compound 1 and about 15 mg QD lenalidomide. In some embodiments, provided methods further comprise administering to a patient in need thereof about 375 m g/m² or about 500 mg/m² rituximab.

In some embodiments, provided methods comprise administering to a patient in need thereof about 500 mg BID Compound 1 and about 2.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 500 mg BID Compound 1 and about 5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 500 mg BID Compound 1 and about 7.5 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 500 mg BID Compound 1 and about 10 mg QD lenalidomide. In some embodiments, provided methods further comprise administering to a patient in need thereof about 375 mg/m² or about 500 mg/m² rituximab.

In some embodiments, provided methods comprise administering to a patient in need thereof about 500 mg BID Compound 1 and about 15 mg QD lenalidomide. In some embodiments, provided methods further comprise administering to a patient in need thereof about 375 mg/m² or about 500 mg/m² rituximab.

In some embodiments, the combination of Compound 1 and lenalidomide is administered over a period of 28 consecutive days (“a 28-day cycle”). In some embodiments, the combination of Compound 1 and lenalidomide is administered for two, three, four, five or six 28-day cycles. In some embodiments, the combination of Compound 1 and lenalidomide is administered for seven, eight, nine, ten, eleven, twelve or more 28-day cycles. In some embodiments, the combination of Compound 1 and lenalidomide is administered once daily for at least thirteen, at least fourteen, at least fifteen, at least sixteen, at least seventeen, at least eighteen, at least nineteen or at least twenty 28-day cycles. In some embodiments, the combination of Compound 1 and lenalidomide is administered to a patient for the duration of the patient's life. In some embodiments, the combination of Compound 1 and lenalidomide is administered to a patient for one or more 28-day cycles, and either of Compound 1 or lenalidomide is further administered to the patient for one or more additional 28-day cycles. In some embodiments, provided methods further comprise administering to a patient need thereof rituximab on day 1 or 2 of cycle 1. In some embodiments, provided methods further comprise administering to a patient need thereof rituximab on day 1 of cycles 2, 3, 4, 5 and 6. In some embodiments, provided methods comprise administering to a patient need thereof each of Compound 1, lenalidomide and rituximab, wherein each of Compound 1 and lenalidomide is administered to a patient for one or more 28-day cycles and rituximab is administered on day 1 or 2 of cycles 1, 2, 3, 4, 5 and/or 6.

In some embodiments, two adjacent 28-day cycles may be separated by a rest period. Such a rest period may be one, two, three, four, five, six, seven or more days during which the patient is not administered either or both Compound 1 and lenalidomide. In a preferred embodiment, two adjacent 28-day cycles are continuous.

In some embodiments, provided methods comprise administering to a patient in need thereof Compound 1 in combination with lenalidomide, wherein the patient has failed at least one prior therapy.

In some embodiments, the present invention provides a system for treating, stabilizing or lessening the severity of one or more diseases or conditions associated with BTK, the system comprising Compound 1 and lenalidomide. In some embodiments, the system is a kit. In some such embodiments, the kit comprises a pharmaceutical composition comprising Compound 1 and a pharmaceutical composition comprising lenalidomide. In some embodiments, the kit comprises one or more unit doses of Compound 1 in combination with one or more unit doses of lenalidomide. In some embodiments, the kit comprises a pharmaceutical composition comprising Compound 1, a pharmaceutical composition comprising lenalidomide and a pharmaceutical composition comprising rituximab. In some embodiments, the kit comprises one or more unit doses of Compound 1, one or more unit doses of lenalidomide and one or more unit doses of rituximab.

In some embodiments, the kit comprises two 125 mg doses of Compound 1 and one 2.5 mg dose of lenalidomide. In some embodiments, the kit comprises two 125 mg doses of Compound 1 and one 5 mg dose of lenalidomide. In some embodiments, the kit comprises two 125 mg doses of Compound 1 and one 7.5 mg dose of lenalidomide. In some embodiments, the kit comprises two 125 mg doses of Compound 1 and one 10 mg dose of lenalidomide. In some embodiments, the kit comprises two 125 mg doses of Compound 1 and one 15 mg dose of lenalidomide.

In some embodiments, the kit comprises two 250 mg doses of Compound 1 and one 2.5 mg dose of lenalidomide. In some embodiments, the kit comprises two 250 mg doses of Compound 1 and one 5 mg dose of lenalidomide. In some embodiments, the kit comprises two 250 mg doses of Compound 1 and one 7.5 mg dose of lenalidomide. In some embodiments, the kit comprises two 250 mg doses of Compound 1 and one 10 mg dose of lenalidomide. In some embodiments, the kit comprises two 250 mg doses of Compound 1 and one 15 mg dose of lenalidomide.

In some embodiments, the kit comprises two 375 mg doses of Compound 1 and one 2.5 mg dose of lenalidomide. In some embodiments, the kit comprises two 375 mg doses of Compound 1 and one 5 mg dose of lenalidomide. In some embodiments, the kit comprises two 375 mg doses of Compound 1 and one 7.5 mg dose of lenalidomide. In some embodiments, the kit comprises two 375 mg doses of Compound 1 and one 10 mg dose of lenalidomide. In some embodiments, the kit comprises two 375 mg doses of Compound 1 and one 15 mg dose of lenalidomide.

In some embodiments, the kit comprises two 500 mg doses of Compound 1 and one 2.5 mg dose of lenalidomide. In some embodiments, the kit comprises two 500 mg doses of Compound 1 and one 5 mg dose of lenalidomide. In some embodiments, the kit comprises two 500 mg doses of Compound 1 and one 7.5 mg dose of lenalidomide. In some embodiments, the kit comprises two 500 mg doses of Compound 1 and one 10 mg dose of lenalidomide. In some embodiments, the kit comprises two 500 mg doses of Compound 1 and one 15 mg dose of lenalidomide.

In some embodiments, a kit comprises seven (7) daily doses of Compound 1 and lenalidomide. In some such embodiments, a kit comprises fourteen (14) 125 mg doses of Compound 1 and seven (7) 2.5 mg doses of lenalidomide. In some such embodiments, a kit comprises fourteen (14) 125 mg doses of Compound 1 and seven (7) 5 mg doses of lenalidomide. In some such embodiments, a kit comprises fourteen (14) 125 mg doses of Compound 1 and seven (7) 7.5 mg doses of lenalidomide. In some such embodiments, a kit comprises fourteen (14) 125 mg doses of Compound 1 and seven (7) 10 mg doses of lenalidomide. In some such embodiments, a kit comprises fourteen (14) 125 mg doses of Compound 1 and seven (7) 15 mg doses of lenalidomide.

In some such embodiments, a kit comprises fourteen (14) 250 mg doses of Compound 1 and seven (7) 2.5 mg doses of lenalidomide. In some such embodiments, a kit comprises fourteen (14) 250 mg doses of Compound 1 and seven (7) 5 mg doses of lenalidomide. In some such embodiments, a kit comprises fourteen (14) 250 mg doses of Compound 1 and seven (7) 7.5 mg doses of lenalidomide. In some such embodiments, a kit comprises fourteen (14) 250 mg doses of Compound 1 and seven (7) 10 mg doses of lenalidomide. In some such embodiments, a kit comprises fourteen (14) 250 mg doses of Compound 1 and seven (7) 15 mg doses of lenalidomide.

In some such embodiments, a kit comprises fourteen (14) 375 mg doses of Compound 1 and seven (7) 2.5 mg doses of lenalidomide. In some such embodiments, a kit comprises fourteen (14) 375 mg doses of Compound 1 and seven (7) 5 mg doses of lenalidomide. In some such embodiments, a kit comprises fourteen (14) 375 mg doses of Compound 1 and seven (7) 7.5 mg doses of lenalidomide. In some such embodiments, a kit comprises fourteen (14) 375 mg doses of Compound 1 and seven (7) 10 mg doses of lenalidomide. In some such embodiments, a kit comprises fourteen (14) 375 mg doses of Compound 1 and seven (7) 15 mg doses of lenalidomide.

In some embodiments, a kit comprises fourteen (14) 500 mg doses of Compound 1 and seven (7) 2.5 mg doses of lenalidomide. In some embodiments, a kit comprises fourteen (14) 500 mg doses of Compound 1 and seven (7) 5 mg doses of lenalidomide. In some embodiments, a kit comprises fourteen (14) 500 mg doses of Compound 1 and seven (7) 7.5 mg doses of lenalidomide. In some embodiments, a kit comprises fourteen (14) 500 mg doses of Compound 1 and seven (7) 10 mg doses of lenalidomide. In some embodiments, a kit comprises fourteen (14) 500 mg doses of Compound 1 and seven (7) 15 mg doses of lenalidomide.

In some embodiments, a kit comprises 28 daily doses of Compound 1 and lenalidomide. In some embodiments, a kit comprises fifty-six (56) 125 mg doses of Compound 1 and twenty-eight (28) 2.5 mg doses of lenalidomide. In some embodiments, a kit comprises fifty-six (56) 125 mg doses of Compound 1 and twenty-eight (28) 5 mg doses of lenalidomide. In some embodiments, a kit comprises fifty-six (56) 125 mg doses of Compound 1 and twenty-eight (28) 7.5 mg doses of lenalidomide. In some embodiments, a kit comprises fifty-six (56) 125 mg doses of Compound 1 and twenty-eight (28) 10 mg doses of lenalidomide. In some embodiments, a kit comprises fifty-six (56) 125 mg doses of Compound 1 and twenty-eight (28) 15 mg doses of lenalidomide. In some embodiments, the kit further comprises one 100 mg/10 mL vial of rituximab. In some embodiments, the kit further comprises one 500 mg/50 mL vial of rituximab.

In some embodiments, a kit comprises fifty-six (56) 250 mg doses of Compound 1 and twenty-eight (28) 2.5 mg doses of lenalidomide. In some embodiments, a kit comprises fifty-six (56) 250 mg doses of Compound 1 and twenty-eight (28) 5 mg doses of lenalidomide. In some embodiments, a kit comprises fifty-six (56) 250 mg doses of Compound 1 and twenty-eight (28) 7.5 mg doses of lenalidomide. In some embodiments, a kit comprises fifty-six (56) 250 mg doses of Compound 1 and twenty-eight (28) 10 mg doses of lenalidomide. In some embodiments, a kit comprises fifty-six (56) 250 mg doses of Compound 1 and twenty-eight (28) 15 mg doses of lenalidomide. In some embodiments, the kit further comprises one 100 mg/10 mL vial of rituximab. In some embodiments, the kit further comprises one 500 mg/50 mL vial of rituximab.

In some embodiments, a kit comprises fifty-six (56) 375 mg doses of Compound 1 and twenty-eight (28) 2.5 mg doses of lenalidomide. In some embodiments, a kit comprises fifty-six (56) 375 mg doses of Compound 1 and twenty-eight (28) 5 mg doses of lenalidomide. In some embodiments, a kit comprises fifty-six (56) 375 mg doses of Compound 1 and twenty-eight (28) 7.5 mg doses of lenalidomide. In some embodiments, a kit comprises fifty-six (56) 375 mg doses of Compound 1 and twenty-eight (28) 10 mg doses of lenalidomide. In some embodiments, a kit comprises fifty-six (56) 375 mg doses of Compound 1 and twenty-eight (28) 15 mg doses of lenalidomide. In some embodiments, the kit further comprises one 100 mg/10 mL vial of rituximab. In some embodiments, the kit further comprises one 500 mg/50 mL vial of rituximab.

In some embodiments, a kit comprises fifty-six (56) 500 mg doses of Compound 1 and twenty-eight (28) 2.5 mg doses of lenalidomide. In some embodiments, a kit comprises fifty-six (56) 500 mg doses of Compound 1 and twenty-eight (28) 5 mg doses of lenalidomide. In some embodiments, a kit comprises fifty-six (56) 500 mg doses of Compound 1 and twenty-eight (28) 7.5 mg doses of lenalidomide. In some embodiments, a kit comprises fifty-six (56) 500 mg doses of Compound 1 and twenty-eight (28) 10 mg doses of lenalidomide. In some embodiments, a kit comprises fifty-six (56) 500 mg doses of Compound 1 and twenty-eight (28) 15 mg doses of lenalidomide. In some embodiments, the kit further comprises one 100 mg/10 mL vial of rituximab. In some embodiments, the kit further comprises one 500 mg/50 mL vial of rituximab.

### IV. Formulations Comprising Compound 1

As described above, provided methods comprise administering to a patient in need thereof a pharmaceutically acceptable composition comprising Compound 1, wherein the pharmaceutically acceptable composition is an oral dosage form. In some embodiments, the pharmaceutically acceptable composition is formulated as a capsule.

In certain embodiments, provided methods comprise administering to a patient in need thereof a pharmaceutically acceptable composition which comprises Compound 1, and one or more pharmaceutically acceptable excipients, such as, for example, binders, film coatings, diluents, disintegrants, surfactants (wetting agents), lubricants and glidants (adsorbents), or combinations thereof. One skilled in the art will readily appreciate that the category under which a particular component is listed is not intended to be limiting; in some cases a particular component might appropriately fit in more than one category. Also, as will be appreciated, the same component can sometimes perform different functions, or can perform more than one function, in the context of a particular formulation, for example depending upon the amount of the ingredient and/or the presence of other ingredients and/or active compound(s). In some embodiments, the pharmaceutically acceptable composition is a blended powder.

### i. Binders and Diluents

Pharmaceutical compositions for use in the present invention may comprise one or more binders. Binders are used in the formulation of solid oral dosage forms to hold the active pharmaceutical ingredient and inactive ingredients together in a cohesive mix. In some embodiments, pharmaceutical compositions of the present invention comprise about 5% to about 50% (w/w) of one or more binders and/or diluents. In some embodiments, pharmaceutical compositions of the present invention comprise about 20% (w/w) of one or more binders and/or diluents. Suitable binders and/or diluents (also referred to as “fillers”) are known in the art. Representative binders and/or diluents include, but are not limited to, starches such as celluloses (low molecular weight HPC (hydroxypropyl cellulose), microcrystalline cellulose (e.g., Avicel), low molecular weight HPMC (hydroxypropyl methylcellulose), low molecular weight carboxymethyl cellulose, ethylcellulose), sugars such as lactose (i.e. lactose monohydrate), sucrose, dextrose, fructose, maltose, glucose, and polyols such as sorbitol, mannitol, lactitol, malitol and xylitol, or a combination thereof. In some embodiments, a provided composition comprises a binder of microcrystalline cellulose and/or lactose monohydrate.

### ii. Disintegrants

Pharmaceutical compositions for use in the present invention may further comprise one or more disintegrants. Suitable disintegrants are known in the art and include, but are not limited to, agar, calcium carbonate, sodium carbonate, sodium bicarbonate, cross-linked sodium carboxymethyl cellulose (croscarmellose sodium), sodium carboxymethyl starch (sodium starch glycolate), microcrystalline cellulose, or a combination thereof. In some embodiments, provided formulations comprise from about 1%, to about 25% disintegrant, based upon total weight of the formulation.

### iii. Surfactants

Surfactants, also referred to as bioavailability enhancers, are well known in the art and typically facilitate drug release and absorption by enhancing the solubility of poorly-soluble drugs. Representative surfactants include, but are not limited to, poloxamers, polyoxyethylene ethers, polyoxyethylene fatty acid esters, polyethylene glycol fatty acid esters, polyoxyethylene hydrogenated castor oil, polyoxyethylene alkyl ether, polysorbates, and combinations thereof. In certain embodiments, the surfactant is a poloxamer. In some such embodiments, the poloxamer is poloxamer 407. In some embodiments, compositions for use in the present invention comprise from about 1% to about 30% by weight of surfactant, based upon total weight of the blended powder.

### iv. Lubricants

Pharmaceutical compositions of the present invention may further comprise one or more lubricants. Lubricants are agents added in small quantities to formulations to improve certain processing characteristics. Lubricants prevent the formulation mixture from sticking to the compression machinery and enhance product flow by reducing interparticulate friction. Representative lubricants include, but are not limited to, magnesium stearate, glyceryl behenate, sodium stearyl fumarate and fatty acids (i.e. palmitic and stearic acids). In certain embodiments, a lubricant is magnesium stearate. In some embodiments, provided formulations comprise from about 0.2% to about 3% lubricant, based upon total weight of given formulation.

### v. Glidants

Pharmaceutical compositions of the present invention may further comprise one or more glidants. Representative glidants include, but are not limited to, silicas (i.e. fumed silica), microcrystalline celluloses, starches (i.e. corn starch) and carbonates (i.e. calcium carbonate and magnesium carbonate). In some embodiments, provided formulations comprise from about 0.2% to about 3% glidant, based upon total weight of given formulation.

### vi. N-(3-(5-fluoro-2-(4-(2-methoxyethoxyl)phenylamino)pyrimidin-4-ylamino)phenyl)acrylamide besylate

As described above, the present invention provides a method of treating a disease or disorder selected from chronic lymphocytic leukemia and small lymphocytic lymphoma, the method comprising administering to a patient in need thereof Compound 1 in combination with lenalidomide. The besylate salt of Compound 1, N-(3-(5-fluoro-2-(4-(2-methoxyethoxyl)phenylamino)pyrimidin-4-ylamino)phenyl)acrylamide benzenesulfonic acid salt, has recently been identified and is currently in clinical trials as monotherapy in subjects with relapsed or refractory B-cell non-Hodgkin's lymphoma (B-NHL), chronic lymphocytic leukemia (CLL) and Waldenstrom's macroglobulinemia (WM). Thus, in some embodiments, provided methods comprise administering to a patient in need thereof a besylate salt of Compound 1.

In some embodiments, provided methods comprise administering to a patient in need thereof a pharmaceutically acceptable composition comprising from about 5% to about 60% of the besylate salt of Compound 1, based upon total weight of the formulation. In some embodiments, provided methods comprise administering to a patient in need thereof a pharmaceutically acceptable composition comprising from about 5% to about 15% or about 7% to about 15% or about 7% to about 10% or about 9% to about 12% of the besylate salt of Compound 1, based upon total weight of the composition. In some embodiments, provided methods comprise administering to a patient in need thereof a pharmaceutically acceptable composition comprising from about 25% to about 75% or about 30% to about 60% or about 40% to about 50% or about 40% to about 45% of the besylate salt of Compound 1, based upon total weight of the formulation. In certain embodiments, provided methods comprise administering to a patient in need thereof a pharmaceutically acceptable composition comprising from about 8%, about 9%, about 10%, about 11%, about 12%, about 13%, about 20%, about 30%, about 40%, about 41%, about 42%, about 43%, about 44%, about 45%, about 50%, about 60%, about 70%, or about 75% of the besylate salt of Compound 1, based upon total weight of given composition or formulation.

In some such embodiments, provided methods comprise administering to a patient in need thereof a pharmaceutical composition comprising a unit dose of Compound 1, wherein Compound 1 is in the form of a besylate salt. In some such embodiments, the unit dose is an amount sufficient to provide about 25 mg, about 50 mg, about 75 mg, about 100 mg, about 125 mg, about 150 mg, about 175 mg, about 200 mg, about 225 mg or about 250 mg of the free base of Compound 1. In some embodiments, the pharmaceutical composition comprising the besylate salt of Compound 1 is a solid oral dosage form.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof Compound 1 in combination with lenalidomide, wherein Compound 1 is administered as the besylate salt. In some such embodiments, the besylate salt of Compound 1 is administered in the form of a composition comprising one or more pharmaceutically acceptable excipients selected from binders, film coatings, diluents, disintegrants, surfactants, lubricants and glidants. In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof each of Compound 1, lenalidomide and rituximab, wherein Compound 1 is administered as the besylate salt. In some such embodiments, the besylate salt of Compound 1 is administered in the form of a composition comprising one or more pharmaceutically acceptable excipients selected from binders, film coatings, diluents, disintegrants, surfactants, lubricants and glidants.

In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof a pharmaceutical composition comprising the besylate salt of Compound 1 in combination with lenalidomide, wherein the amount of besylate salt of Compound 1 is sufficient to deliver about 125 mg, about 250 mg, about 325 mg, about 375 mg, about 400 mg, about 500 mg, about 625 mg, about 750 mg, about 1000 mg or about 1250 mg of the free base of Compound 1. In some such embodiments, the pharmaceutical composition further comprises one or more pharmaceutically acceptable excipients selected from binders, film coating, diluents, disintegrants, surfactants, lubricants and glidants. In some such embodiments, the pharmaceutical composition comprises one or more pharmaceutically acceptable excipients selected from microcrystalline cellulose, lactose monohydrate, sodium starch, poloxamer 407, fumed silica and magnesium stearate. In some embodiments, the present invention provides a method of treating, stabilizing or lessening the severity or progression of a disease or disorder selected from the group consisting of chronic lymphocytic leukemia and small lymphocytic lymphoma, the method comprising administering to a patient in need thereof a pharmaceutical compositions comprising each of the besylate salt of Compound 1 (i.e., Compound 1 besylate), lenalidomide and rituximab, wherein the amount of besylate salt of Compound 1 is sufficient to deliver about 125 mg, about 250 mg, about 325 mg, about 375 mg, about 400 mg, about 500 mg, about 625 mg, about 750 mg, about 1000 mg or about 1250 mg of the free base of Compound 1.

### V. Process for Preparing Pharmaceutical Compositions Comprising Compound 1 Dry Blend Process:

Milled N-(3-(5-fluoro-2-(4-(2-methoxyethoxyl)phenylamino)pyrimidin-4-ylamino)phenyl)acrylamide besylate, milled microcrystalline cellulose, milled sodium starch glycolate, milled lactose monohydrate, milled poloxamer 407, and sieved fumed silica are weighed and mechanically blended. An intragranular portion of sieved magnesium stearate (2.0%, per Table 1, below) is added to the blender and the formulation blended. This blended formulation is then roller compacted, milled, and then blended. The blended formulation is additionally roller compacted, milled and then blended. The remainder or extragranular portion of the magnesium stearate (0.5%, per Table 1, below) is added and the final formulation is blended. Capsules are either mechanically filled or manually filled via the flood fill method.

All features of each of the aspects of the invention apply to all other aspects mutatis mutandis. Each of the references referred to herein, including but not limited to patents, patent applications and journal articles, is incorporated by reference herein as though fully set forth in its entirety.

In order that the invention described herein may be more fully understood, the following examples are set forth. It should be understood that these examples are for illustrative purposes only and are not to be construed as limiting this invention in any manner.

### EXEMPLIFICATION

### Example 1

### Dose Escalation Study

N-(3-(5-fluoro-2-(4-(2-methoxyethoxyl)phenylamino)pyrimidin-4-ylamino)phenyl)acrylamide besylate is a chemically synthesized small molecule substituted pyrimidine developed as the benzenesulfonic acid salt and is a white to off-white crystalline powder. N-(3-(5-fluoro-2-(4-(2-methoxyethoxyl)phenylamino)pyrimidin-4-ylamino)phenyl)acrylamide besylate is an oral, potent (IC₅₀<0.5 nM) and selective small molecule inhibitor of Btk. N-(3-(5-fluoro-2-(4-(2-methoxyethoxyl)phenylamino)pyrimidin-4-ylamino)phenyl)acrylamide besylate exhibits solubility of approximately 0.16 mg/mL in water and a maximum aqueous solubility of 0.40 mg/mL at approximately pH 3.0. The solubility of N-(3-(5-fluoro-2-(4-(2-methoxyethoxyl)phenylamino)pyrimidin-4-ylamino)phenyl)acrylamide besylate in ethanol is approximately 10 mg/mL. N-(3-(5-fluoro-2-(4-(2-methoxyethoxyl)phenylamino)pyrimidin-4-ylamino)phenyl)acrylamide besylate exhibits no environmental instabilities (i.e. heat, acid, base) that require special handling.

N-(3-(5-fluoro-2-(4-(2-methoxyethoxyl)phenylamino)pyrimidin-4-ylamino)phenyl)acrylamide besylate was formulated into capsules containing the components and quantities listed in Table 1 to obtain the study drug. The capsules listed in Table 1 will be administered during the dose escalation and expansion cohort studies.

TABLE 1
Components of N-(3-(5-fluoro-2-(4-(2-methoxyethoxy)phenylamino)
pyrimidin-4-ylamino)phenyl)acrylamide besylate capsules

| Component | Amount per 125 mg Capsule |
|---|---|
| Capsule shell | 1, size 0 |
| | white capsule |
| N-(3-(5-fluoro-2-(4-(2- | 174.30 mg (125 mg free base) |
| methoxyethoxy)phenylamino) | |
| pyrimidin-4-ylamino)phenyl) | |
| acrylamide besylate | |
| Microcrystalline cellulose | 101.68 mg |
| Lactose monohydrate | 41.50 mg |
| Sodium starch glycolate | 41.50 mg |
| Poloxamer 407 | 41.50 mg |
| Fumed silica | 4.15 mg |
| Magnesium stearate | 10.38 mg‡ |

‡2.0% (8.30 mg) intragranular; 0.5% (2.08 mg) extragranular.

Lenalidomide, also known as 3-(4-amino-1-oxo-1,3-dihydro-2H-isoindol-2-yl)piperidine-2,6-dione, has the following structure:

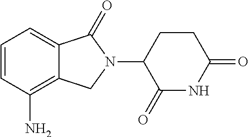 [SMILES: Nc1cccc2c1CN(C1CCC(=O)NC1=O)C2=O]

Lenalidomide is an off-white to pale yellow solid powder and is commercially available as 2.5 mg, 5 mg, 10 mg, 15 mg and 25 mg capsules from Celgene Corporation. Each capsule of lenalidomide contains lactose anhydrous, microcrystalline cellulose, croscarmellose sodium and magnesium stearate.

Lenalidomide will be formulated into capsules containing the components and quantities listed in Table 2 to obtain the study drug. Capsules listed in Table 2 will be administered during the dose escalation and expansion cohort studies according to the dose needed and represent the clinical supply image. As commercial image product may also be used, the components and quantities for commercial image product are listed in Table 3.

TABLE 2
Components of lenalidomide capsules—Clinical Supply Image

| | 2.5 mg | | | | |
|---|---|---|---|---|---|
| | (1%) | 5 mg | 10 mg | 15 mg | 25 mg |
| | Capsule | Capsule | Capsule | Capsule | Capsule |
| Ingredient | Theoretical Weight per Capsule (mg) | | | | |
| Lenalidomide^{a} | 2.5 | 5.0 | 10.0 | 15.0 | 25.0 |
| Lactose | 73.5 | 147.0 | 294.0 | 289.0 | 200.0 |
| Anhydrous^{b} | | | | | |
| Microcrystalline | 20.0 | 40.0 | 80.0 | 80.0 | 159.0 |
| Cellulose | | | | | |
| Croscarmellose | 3.0 | 6.0 | 12.0 | 12.0 | 12.0 |
| Sodium | | | | | |
| Magnesium | 1.0 | 2.0 | 4.0 | 4.0 | 4.0 |
| Stearate | | | | | |
| Total Fill Weight | 100.0 | 200.0 | 400.0 | 400.0 | 400.0 |
| White Capsule | 1 | — | — | — | — |
| Shells (Size 4) | Capsule | | | | |
| White Capsule | | 1 | — | — | — |
| Shells (Size 2) | | Capsule | | | |
| White Capsule | | — | 1 | 1 | 1 |
| Shells (Size 0) | | | Capsule | Capsule | Capsule |

^{a}The weight of lenalidomide is adjusted for assay and water content.
^{b}The weight of Lactose Anhydrous, NF/EP is adjusted to achieve the target total blend weight for the batch.

TABLE 3
Components of lenalidomide capsules - Commercial Supply Image

| | 2.5 mg | 5 mg | 10 mg | 15 mg | 20 mg | 25 mg |
|---|---|---|---|---|---|---|
| | Capsule | Capsule | Capsule | Capsule | Capsule | Capsule |
| Ingredient | Theoretical Weight per Capsule (mg) | | | | | |
| Lenalidomide^{a} | 2.5 | 5.0 | 10.0 | 15.0 | 20.0 | 244.5 |
| Lactose Anhydrous^{b} | 73.5 | 147.0 | 294.0 | 289.0 | 244.5 | 120.5 |
| Microcrystalline | 20.0 | 40.0 | 80.0 | 80.0 | 120.5 | 12.0 |
| Cellulose | | | | | | |
| Croscarmellose | 3.0 | 6.0 | 12.0 | 12.0 | 12.0 | 3.0 |
| Sodium | | | | | | |
| Magnesium Stearate | 1.0 | 2.0 | 4.0 | 4.0 | 3.0 | 400.0 |
| Total Fill Weight | 100.0 | 200.0 | 400.0 | 400.0 | 400.0 | — |
| White body/Blue | 1 Capsule | — | — | — | — | — |
| Green Cap Capsule | | | | | | |
| Shells (Size 4) | | | | | | |
| Imprinted with Black | | | | | | |
| Ink^{c} | | | | | | |
| White Capsule Shells | — | 1 Capsule | — | — | — | |
| (Size 2) Imprinted with | | | | | | |
| Black Ink^{c} | | | | | | |
| Pale Yellow | — | — | 1 Capsule | — | — | |
| Body/Blue Green Cap | | | | | | |
| Capsule Shells (Size 0) | | | | | | |
| Imprinted with Black | | | | | | |
| Ink^{c} | | | | | | |
| White Body/Powder | — | — | — | 1 Capsule | — | |
| Blue Cap Capsule | | | | | | |
| Shells (Size 0) | | | | | | |
| Imprinted with Black | | | | | | |
| Ink^{c} | | | | | | |
| Blue Body/Blue Green | — | — | — | — | 1 Capsule | |
| Cap Capsule Shells | | | | | | |
| (Size 0) Imprinted with | | | | | | |
| Black Ink^{c} | | | | | | |
| White Capsule Shells | — | — | — | — | — | 1 Capsule |
| (Size 0) Imprinted with | | | | | | |
| Black Ink^{c} | | | | | | |

^{a}The weight of lenalidomide is adjusted for assay and water content.
^{b}The weight of Lactose Anhydrous, NF/EP is adjusted to achieve the target total blend weight for the batch.
^{c}The capsule shells are supplied by Capsugel.

Study Design

Subjects with relapsed or refractory CLL or SLL who failed at least one prior treatment regimen were enrolled in a “3+3” dose escalation and expansion study to determine the Not Tolerated Dose (NTD), the Optimal Biologic Effect dose (OBE) and the Maximum Tolerated Dose (MTD) of the combination of Compound 1 and lenalidomide. Approximately 42 patients will be enrolled in the study.

Three (3) dose levels and three proposed dose levels were defined as outlined in Table 4.

TABLE 4
Study Dosing Schema for Escalating Dose Portion of Study

| | N-(3-(5-fluoro-2-(4-(2- | | |
|---|---|---|---|
| | methoxyethoxy)phenylamino) | | |
| | pyrimidin-4-ylamino)phenyl) | | |
| COHORT | acrylamide besylate | Lenalidomide | |
| 1a* | 375 mg BID | 5 | mg QD |
| 1 | 375 mg BID | 10 | mg QD |
| 2a | 500 mg BID | 5 | mg QD |
| 2 | 500 mg BID | 10 | mg QD |
| 3a* | 500 mg BID | 7.5 | mg QD |
| 3 | 500 mg BID | 15 | mg QD |

*Proposed additional dose levels.

Study treatment was administered in 28-day cycles at specified dose levels as scheduled until disease progression, unacceptable toxicity, or discontinuation for any other reason. Subjects will continue on the starting dose until the preliminary recommended Phase 2 dose (RP2D) is determined, at which point they can be switched to the preliminary RP2D.

Within each cohort, subjects were treated PO (oral) BID (twice daily) with N-(3-(5-fluoro-2-(4-(2-methoxyethoxyl)phenylamino)pyrimidin-4-ylamino)phenyl) acrylamide besylate in combination with lenalidomide during an initial 28-day treatment cycle and were assessed for safety, tolerability and DLT, as well as pharmacokinetic (“PK”), pharmacodynamic (“PD”), and disease response. In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

Within a defined cohort, an investigator can elect to reduce the dose of either, or both, of N-(3-(5-fluoro-2-(4-(2-methoxyethoxyl)phenylamino)pyrimidin-4-ylamino)phenyl)acrylamide besylate and lenalidomide. In each case, an investigator can elect to step down the dose level of N-(3-(5-fluoro-2-(4-(2-methoxyethoxyl)phenylamino)pyrimidin-4-ylamino)phenyl)acrylamide besylate as follows. A patient in cohort 1 can be stepped down from 375 mg BID to 250 mg BID, and then again from 250 mg BID to 125 mg BID N-(3-(5-fluoro-2-(4-(2-methoxyethoxyl)phenylamino)pyrimidin-4-ylamino)phenyl)acrylamide besylate. A patient in cohort 1a can be stepped down from 375 mg BID to 250 mg BID, and then again from 250 mg BID to 125 mg BID N-(3-(5-fluoro-2-(4-(2-methoxyethoxy)phenylamino)pyrimidin-4-ylamino)phenyl)acrylamide besylate. A patient in cohort 2 can be stepped down from 500 mg BID to 375 mg BID, and then again from 375 mg BID to 250 mg BID N-(3-(5-fluoro-2-(4-(2-methoxyethoxyl)phenylamino)pyrimidin-4-ylamino)phenyl)acrylamide besylate. A patient in cohort 2a can be stepped down from 500 mg BID to 375 mg BID, and then again from 375 mg BID to 250 mg BID N-(3-(5-fluoro-2-(4-(2-methoxyethoxy)phenylamino)pyrimidin-4-ylamino)phenyl)acrylamide besylate. A patient in cohort 3a can be stepped down from 500 mg BID to 375 mg BID, and then again from 375 mg BID to 250 mg BID N-(3-(5-fluoro-2-(4-(2-methoxyethoxyl)phenylamino)pyrimidin-4-ylamino)phenyl)acrylamide besylate. A patient in cohort 3 can be stepped down from 500 mg BID to 375 mg BID, and then again from 375 mg BID to 250 mg BID N-(3-(5-fluoro-2-(4-(2-methoxyethoxy)phenylamino)pyrimidin-4-ylamino)phenyl)acrylamide besylate.

An investigator can elect to step down the dose level of lenalidomide. As of Aug. 6, 2013, such dose reduction steps are defined in Table 5:

TABLE 5
Lenalidomide Dose Reduction Steps
Lenalidomide Dose

| | Starting Dose | |
|---|---|---|
| | 10 mg QD | 15 mg QD |
| Dose Reduction 1 | 7.5 mg QD | 10 mg QD |
| Dose Reduction 2 | 5 mg QD | 7.5 mg QD |
| Dose Reduction 3 | Discontinue drug | Discontinue drug |

In some cases, an investigator may elect to step down the dose level of lenalidomide according to Table 6:

TABLE 6
Alternative Lenalidomide Dose Reduction Steps
Lenalidomide Dose

| | Starting Dose | | | |
|---|---|---|---|---|
| | 5 mg QD | 7.5 mg QD | 10 mg QD | 15 mg QD |
| Dose Reduction 1 | 2.5 mg QD | 5 mg QD | 7.5 mg QD | 10 mg QD |
| Dose Reduction 2 | Discontinue | 2.5 mg QD | 5 mg QD | 7.5 mg QD |
| | drug | | | |
| Dose Reduction 3 | n/a | Discontinue | 2.5 mg QD | 5 mg QD |
| | | drug | | |
| Dose Reduction 4 | n/a | n/a | Discontinue | 2.5 mg QD |
| | | | drug | |
| Dose Reduction 5 | n/a | n/a | n/a | Discontinue |
| | | | | drug |

It will be understood that a dose reduction is not required and is provided only as a means of mitigating or alleviating one or more adverse events deemed to be associated with the study drug(s). Any such dose reduction does not change the enrollment of the patient; thus, any patient who has had one or more dose reductions is still enrolled in the cohort to which they were originally assigned.

The dose level at which a patient is enrolled will be based on which cohort is open at the time of enrollment. For the first cycle only, lenalidomide will be administered on days 1 to and N-(3-(5-fluoro-2-(4-(2-methoxyethoxyl)phenylamino)pyrimidin-4-ylamino)phenyl)acrylamide besylate will be administered on days 8-28 of the 28-day cycle. Thereafter, lenalidomide and N-(3-(5-fluoro-2-(4-(2-methoxyethoxyl)phenylamino)pyrimidin-4-ylamino)phenyl)acrylamide besylate will be administered each day of a given 28-day cycle according to Table 4.

A dose level is considered tolerable if zero (0) of three (3) subjects dosed experience a DLT. Dose escalation, via enrollment in the next higher dose, is allowed only if none (0) of the first three (3) subjects enrolled in any cohort experience dose limiting toxicity (DLT). If one (1) of the first three (3) subjects dosed in any cohort experiences a DLT in cycle 1, three (3) more subjects will be enrolled in that dose cohort. A dose will be considered a NTD when two (2) of six (6) DLT-evaluable subjects in that cohort experience a DLT. A MTD will be declared when at least six (6) subjects have been enrolled and safely complete cycle 1 at that dose level. The MTD is defined as the last dose below the NTD with zero (0) or one (1) DLT-evaluable subject experiencing DLT during the first 28-day cycle.

During the dose escalation phase, a decision to enroll the next higher dose cohort will be based on review of safety and DLT-evaluable patients. The OBE dose is defined as follows:
90% target occupancy in five (5) of six (6) subjects at any dose; and/or
no further increase in exposure with increasing doses; and/or
results in a ≧25% increase in absolute lymphocyte count in four (4) of six (6) subjects during the first three 28-day cycles (Cycles 1-3) not assessed as progressive disease (PD); and/or
results in a ≧50% reduction in total lymph node area in at least 50% of subjects at any dose or schedule level.

Subjects will remain on study until the subject discontinues due to disease progression, unacceptable toxicity, withdrawal of consent or any other reason determined by the physician. Preliminary evidence of efficacy will be evaluated.

Results.

“Complete Response” (CR) is defined per IWCLL criteria, 2008. (No LN >1.5 cm, no hepatomegaly, splenomegaly, ALC <4000/uL, normocellular marrow <30% lymphocytes, ANC >1500, Platelet Count >100,000 and Hgb >11.0 g/dL). “Partial Response” (PR) was assessed via IWCLL guidelines (at least 2 of the following criteria—lymph node (LN) decrease≧50%; hepatomegaly decrease≧50%; splenomegaly decrease≧50%; ALC decrease≧50%; and at least 1 of the following—platelet count >100,000; ANC >1500/uL or Hgb >11.0 g/dL). “Nodal Response” is defined as a reduction of total lymph node area by at least 50%. A status of PR is the investigator's assessment based on a physical exam evaluation of lymph nodes, spleen and liver and laboratory values of blood counts. Confirmed PR also includes imaging of tumor lesions by CT scan. Stable disease was determined by repeat CT scans following unconfirmed CR clinical responses.

Cohort 1.

Three subjects were enrolled in cohort 1 and treated with N-(3-(5-fluoro-2-(4-(2-methoxyethoxyl)phenylamino)pyrimidin-4-ylamino)phenyl) acrylamide besylate BID in combination with lenalidomide QD. Of the three subjects initially enrolled in cohort 1, one experienced a DLT (Grade 3 hallucinations). Because the study design does not permit dose escalation (via enrollment in the next higher dose) unless 0/3 or only 1/6 subjects at the current dose experience a DLT during the first 28-day cycle, three additional subjects were enrolled in cohort 1.

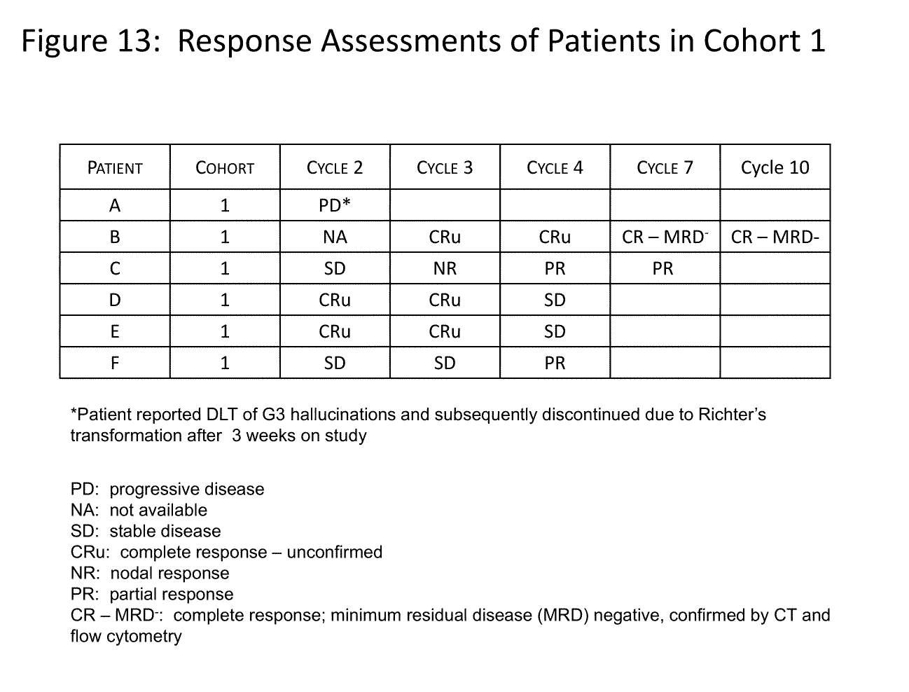

FIG. 13 summarizes the disease status of patients in cohort 1 as of Oct. 16, 2013. Disease status was assessed per the IWCLL Guidelines for the diagnosis and treatment of CLL (Hallek, 2008). Of the six subjects enrolled in cohort 1, subject A exhibited progressive disease during cycle 1 and treatment was discontinued. In cycle 6, subject B exhibited a complete response confirmed with CT scans and bone marrow biopsy. MRD negativity was confirmed via flow cytometry of the bone marrow. Subjects C and F exhibited partial response in cycle 4. Subjects D and E exhibited stable disease in cycle 4.

As of Oct. 16, 2013, Subject B is in cycle 10 with currently both drugs on hold due to an AE of diarrhea. Subject C is in cycle 8 and currently receiving 375 mg BID Compound 1 besylate. Subject D was receiving 375 mg BID Compound 1 besylate through cycle 5. Subject D withdrew consent from the study. Subject E is in cycle 7 and currently receiving the cohort 1 dosage (375 mg BID Compound 1 besylate and 10 mg QD lenalidomide). Subject F is in cycle 6 and receiving a dose of 375 mg BID Compound 1 besylate. Lenalidomide is on hold due to ongoing neutropenia

Cohort 2.

Three subjects were enrolled in cohort 2 and treated with N-(3-(5-fluoro-2-(4-(2-methoxyethoxyl)phenylamino)pyrimidin-4-ylamino)phenyl) acrylamide besylate BID in combination with lenalidomide QD. Of the three subjects initially enrolled in cohort 2, one experienced a DLT during the first cycle (Grade 3 fatigue and Grade 3 rash) and one subject was not DLT evaluable and a replacement was required. Four additional subjects were subsequently enrolled in cohort 2 and began treatment.

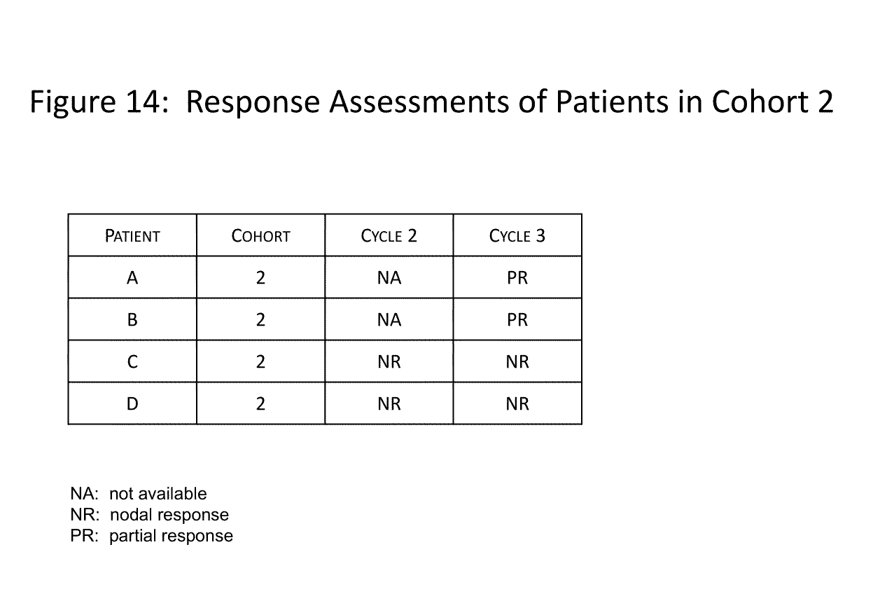

FIG. 14 summarizes the disease status of subjects in cohort 2 as of Oct. 16, 2013. Disease status was assessed per the IWCLL Guidelines for the diagnosis and treatment of CLL (Hallek, 2008). Of the seven subjects enrolled in cohort 2, response data is available for 4 subjects. Subjects A and B exhibited a partial response by Cycle 3. Subjects C and D exhibited a nodal response by Cycle 2. Subjects C and D have not yet met the criteria for partial response due to ongoing lymphocytosis. Efficacy data is unavailable for the remainder of patients enrolled into Cohort 2.

As of Oct. 16, 2013, two subjects have been discontinued during cycle 5 due to disease progression. One subject is in cycle 4 and receiving 500 mg BID Compound 1 besylate and a reduced dose of lenalidomide (5 mg QD) due to persistent thrombocytopenia that was present at baseline. One subject is in cycle 3 and receiving the reduced dose of 375 mg BID Compound 1 besylate; lenalidomide is on hold due to an adverse event of fatigue. One subject is in cycle 3 and receiving 500 mg BID Compound 1 besylate; lenalidomide is on hold due to an adverse event of neutropenia. One additional subject enrolled in cohort 2 was discontinued due to disease progression during cycle 2.

Cohort 2a.

As of Nov. 7, 2013, one patient was enrolled in cohort 2a and is currently in cycle 1 of treatment.

Expansion Cohorts.

After completion of observation for DLTs in the dose escalation study, the accumulated safety, PK, and PD data will be evaluated to select a preliminary RP2D. The preliminary RP2D will be evaluated in expanded cohorts of 24 subjects or a more complete safety profile and further preliminary evaluation of efficacy. If less than 9 of 24 subjects experience DLTs, then this dose level will be declared the RP2D to be used in further studies. If DLTs are experienced in greater than or equal to 9 of 24 subjects, this dose will be considered to have exceeded the MTD and the previous highest tolerated dose found in the dose escalation cohort of the study will be evaluated in 24 subjects. The dose level will continue to be reduced in a stepwise fashion until less than 9 of 24 subjects experience DLTs.

In certain instances, the physician-investigator may elect to rest a patient during the study, during which time the patient does not receive treatment. For example, the physician-investigator may elect to rest a patient due to occurrence or recurrence of adverse events. For purposes of clarity, a patient who has been rested is still enrolled in the study until the physician-investigator determines that the patient should not continue treatment, at which time such patients are discontinued from further treatment. In this context, treatment duration refers to the time a patient is enrolled in the study, inclusive of all rest periods, until treatment is discontinued.

Adverse Events.

For all cohorts, dose limiting toxicities (DLTs) are defined as specified adverse events (AEs) that are observed within the first 28-day cycle and deemed to be related to treatment. Hematologic DLTs include Grade 4 anemia or thrombocytopenia by CTCAE (v. 4.03) or by IWCLL criteria, whichever results in the lower blood threshold; Grade 4 neutropenia greater than 5 days despite granulocyte colony-stimulating factor (G-CSF) support; and Grade 3 or higher febrile neutropenia. Non-hematologic DLTs include Grade 4 or higher non-hematologic AEs of any duration during cycle 1; Grade 3 total bilirubin elevation, whether symptomatic or asymptomatic; and any Grade 3 non-hematologic toxicity except nausea, vomiting and diarrhea lasting less than 24 hours following medical therapy; tumor lysis syndrome which does not progress to Grade 4 and resolves in less than 7 days with medical management; and transient, and Grade 3 non-hematologic laboratory anomaly that is asymptomatic and rapidly reversible (returns to baseline or <Grade 1 within 7 days).

Subjects without disease progression at the end of the first 28-day cycle of treatment were eligible to continue receiving N-(3-(5-fluoro-2-(4-(2-methoxyethoxyl)phenylamino)pyrimidin-4-ylamino)phenyl)acrylamide besylate and lenalidomide for additional 28-day cycles until (i) the patient experiences unacceptable toxicity, (ii) the underlying malignancy progresses, (iii) the patient withdraws consent, or (iv) the treating physician-investigator otherwise determines that the patient should not continue treatment.

As of Oct. 16, 2013, subjects in cohort 1 reported incidences of hallucinations (1 patient; cycle 1), necessitating the expansion of cohort 1, sepsis (1 patient; cycle 4), fever (1 patient, cycle 5) and neutropenia (1 patient; cycle 3).

As of Oct. 16, 2013, subjects in cohort 2 reported incidences of sepsis (1 patient; cycle 2), thrombocytopenia (1 patient; cycle 1, present at baseline), febrile neutropenia (1 patient; cycle 2) and neutropenia (1 patient, cycle 1). One subject reported incidences of fatigue and rash (cycle 1), necessitating the expansion of cohort 2.

Of the first thirteen subjects enrolled in cohorts 1 and 2 (6 in cohort 1 and 7 in cohort 2), three reported mild tumor flare, which was resolved for all patients before the end of the second week of treatment.

Btk Occupancy.

The covalent mechanism of action of N-(3-(5-fluoro-2-(4-(2-methoxyethoxyl)phenylamino)pyrimidin-4-ylamino)phenyl)acrylamide besylate allows for development of a covalent probe to detect free, uninhibited Btk in lysates derived from tissue culture, animal tissues, or clinical samples. PBMC lysates were isolated from whole blood samples 30 minutes before dosing, 4 hours or 24 hours post-dose and incubated with the biotinylated covalent probe. Uninhibited Btk was captured by the covalent probe and quantitated by ELISA. Normalization to untreated control sample allowed for determination of the % Btk occupancy.

Btk Target Site Occupancy ELISA:

Cell lysates or spleen homogenates were incubated with 1 μM N¹-(3-(3-(4-(3-acrylamidophenylamino)-5-methylpyrimidin-2-ylamino)phenoxy)propyl)-N⁵-(15-oxo-19-((3 aS,4S,6aR)-2-oxohexahydro-1H-thieno[3,4-d]imidazol-4-yl)-4,7,10-trioxa-14-azanonadecyl)glutaramide (2) in a PBS, 0.05% Tween-20, 1% BSA solution for 1 h at room temperature. Compound 2 has the following structure:

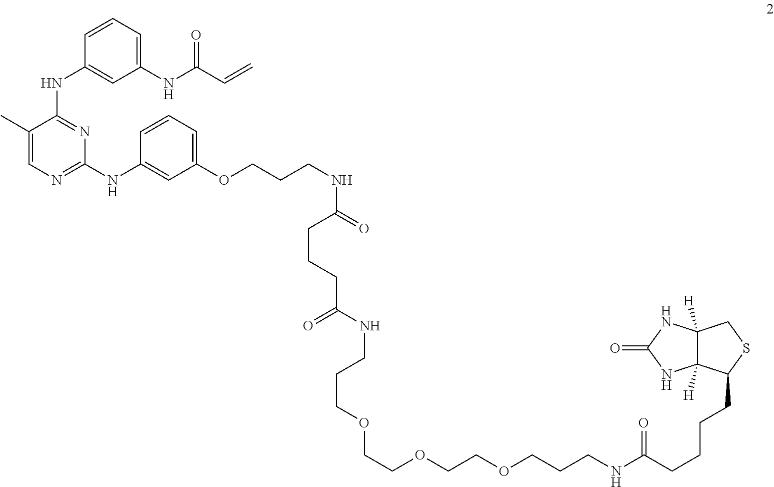 [SMILES: C=CC(=O)Nc1cccc(Nc2nc(Nc3cccc(OCCCNC(=O)CCCC(=O)NCCCOCCOCCOCCCNC(=O)CCCC[C@@H]4SC[C@@H]5NC(=O)N[C@@H]54)c3)ncc2C)c1]

Standards and samples were transferred to a streptavidin-coated 96-well ELISA plate and mixed while shaking for 1 h at room temperature. The a-Btk antibody (BD 611116, 1:1000 dilution in PBS+0.05% Tween-20+0.5% BSA) was then incubated for 1 h at room temperature. After wash, goat a-mouse-HRP (1:5000 dilution in PBS+0.05% Tween-20+0.5% BSA) was added and incubated for 1 h at room temperature. The ELISA was developed with addition of tetramethyl benzidine (TMB) followed by Stop Solution and read at OD 450 nm. The standard curve (11.7-3000 pg/μL) was generated with human full-length recombinant Btk protein and plotted using a 4 parameter curve fit in Gen5 software. Uninhibited Btk detected from samples was normalized to μg total protein as determined by BCA protein analysis (Pierce Cat. 23225).

### Example 2

### Cell Titer Glo Combination Assay

The combination of N-(3-(5-fluoro-2-(4-(2-methoxyethoxyl)phenylamino)pyrimidin-4-ylamino)phenyl)acrylamide besylate (Compound 1 besylate) and lenalidomide was assayed in four model B-cell cancer cell lines to ascertain whether the combination exhibited any synergistic effects.

DLBCL cell lines OCI-LY-10, WSU-DLCL2, Riva and TMD-8 were grown in RMPI+10% FBS medium and plated at 10000 cells/well in 96 well plates. Each cell line is plated in multiple 96 well plates at 90 μL/well.

Based on an initial compound viability assay for each cell line, the range of compound concentration used for each cell line was determined so that maximum and minimum cell viability were achieved with an even spread of cell viability. A stock solution of Compound 1 besylate in DMSO was prepared at 10 mM. The 10 mM stock solution was diluted 3×-10× to between 3333 and 1000 nM and further diluted 3-fold in a 10 point dilution series in DMSO. The dilution series was diluted 50× in RMPI+10% FBS media and 5 μL of Compound 1 besylate was added to 95 μL of cell culture, resulting in a 1:1000 dilution.

Lenalidomide was prepared as a 10 mM stock solution in DMSO and diluted in DMSO to 1000× of the desired final concentration. The dilution series was diluted 200× in RMPI+10% FBS media and 5 μL of lenalidomide was added to 95 μL of cell culture, resulting in a 1:1000 dilution. The final concentration of lenalidomide ranged from 41-10000 nM.

For the combination assays, one concentration of lenalidomide was added to a triplicate dilution series of Compound 1 besylate. At least three, and up to five, different concentrations of lenalidomide were assayed in combination with a triplicate dilution series of Compound 1 besylate. Compound 1 besylate and lenalidomide were applied concomitantly to the cells the day after plating.

72 hours after drug administration, the number of viable cells was determined using CellTiter-Glo according to manufacturer's instructions. Briefly, cells and CellTiter-Glo are equilibrated to room temperature. CellTiter-Glo is added to the cells 1:1 (v/v) and the assay was performed. Results were read on a Molecular Devices spectramax L luminescence detector.

Luminescence readings were normalized as a percent of the average control (DMSO treated) luminescence reading. The expected combination viability was calculated using the formula:

(Compound 1 besylate % survival fraction)×(lenalidomide % survival fraction)/100

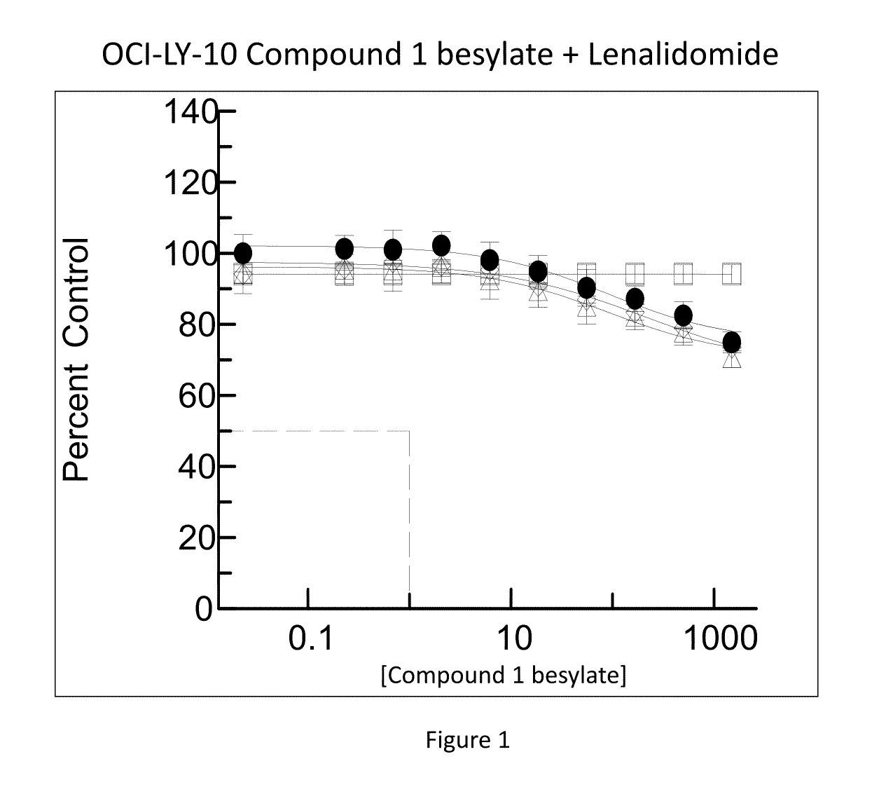

Microsoft Excel with the XLfit plugin was used to plot the viability curves. Synergy (“volcano”) plots were created by plotting the difference between observed and expected combination treatment values. Results are presented in FIGS. 1-12. The graphs depicted in the Figures are exemplary unless otherwise indicated.

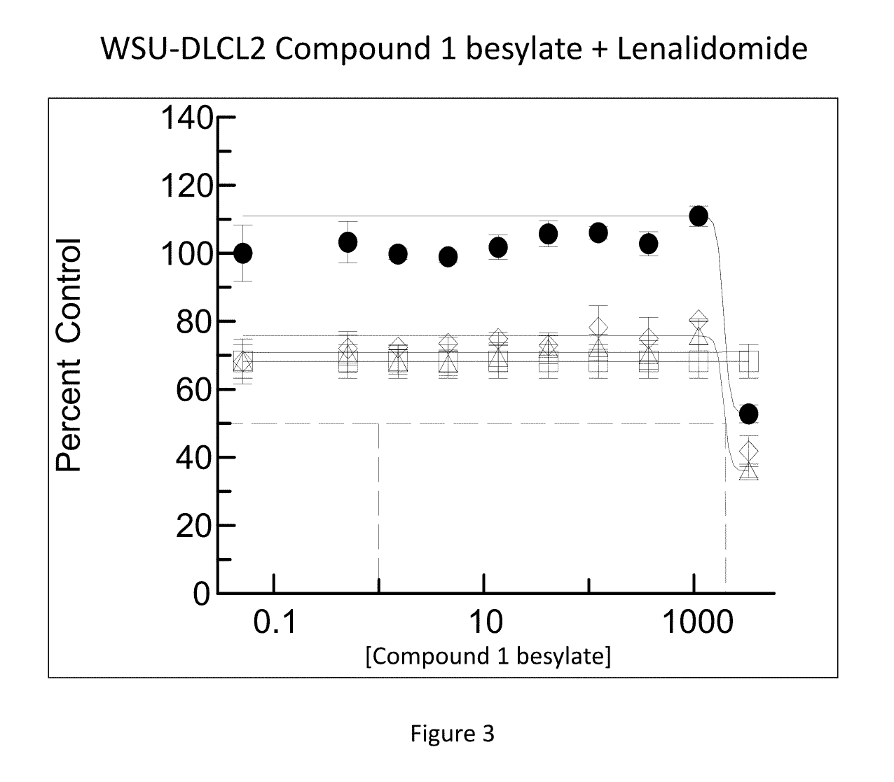

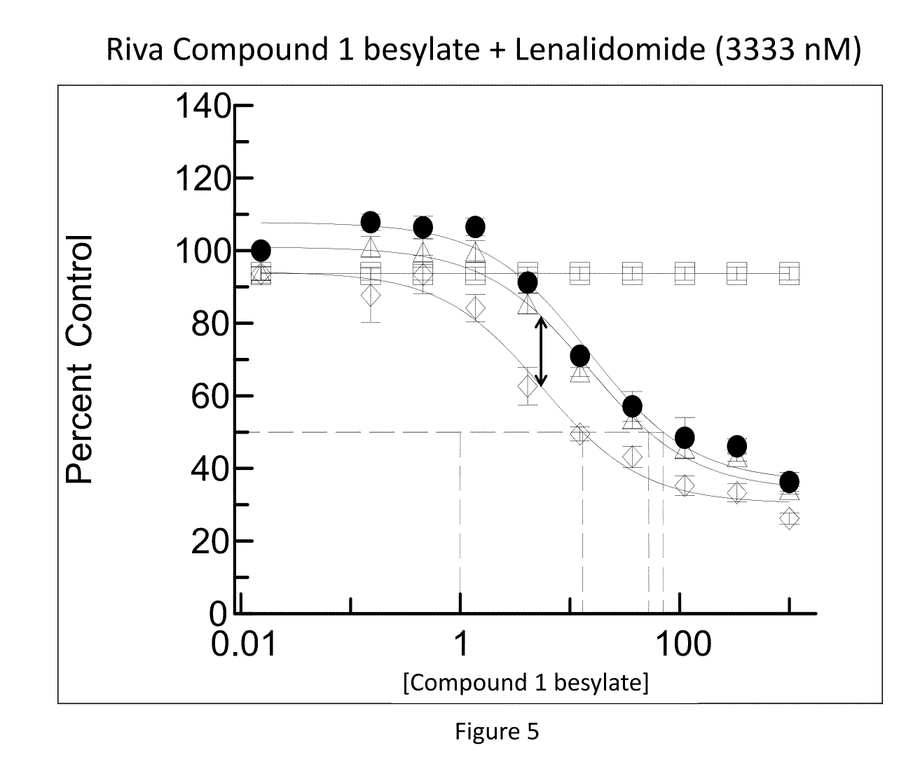

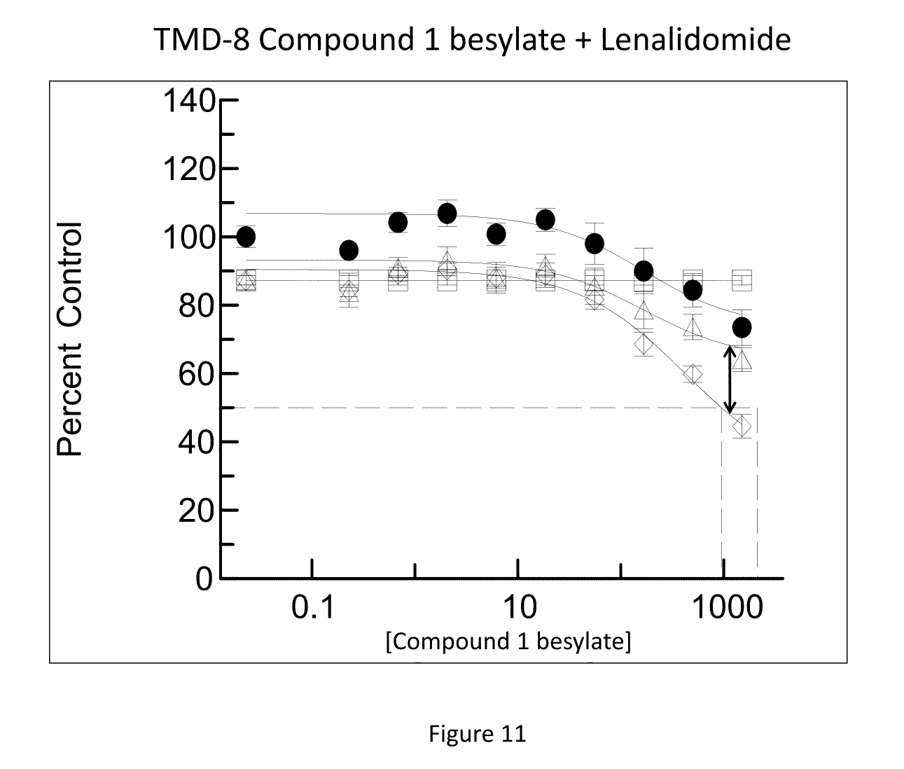

The dose response curves in FIGS. 1, 3, 5, 7, 9, and 11 depict the results of the combination of Compound 1 besylate and lenalidomide in cell lines OCI-LY-10 (FIG. 1), WSU-DLCL2 (FIG. 3), Riva (FIGS. 5, 7 and 9) and TMD-8 (FIG. 11). The response of cells to increasing concentrations of Compound 1 besylate is indicated by . The response of cells to a test concentration of lenalidomide is indicated by □. The expected (or calculated) activity of the combination is indicated by Δ. The observed activity of the combination is indicated by ⋄. The combination is additive when the expected or calculated activity of the combination is equal to the observed activity (i.e., when the lines represented by Δ and ⋄ overlap).

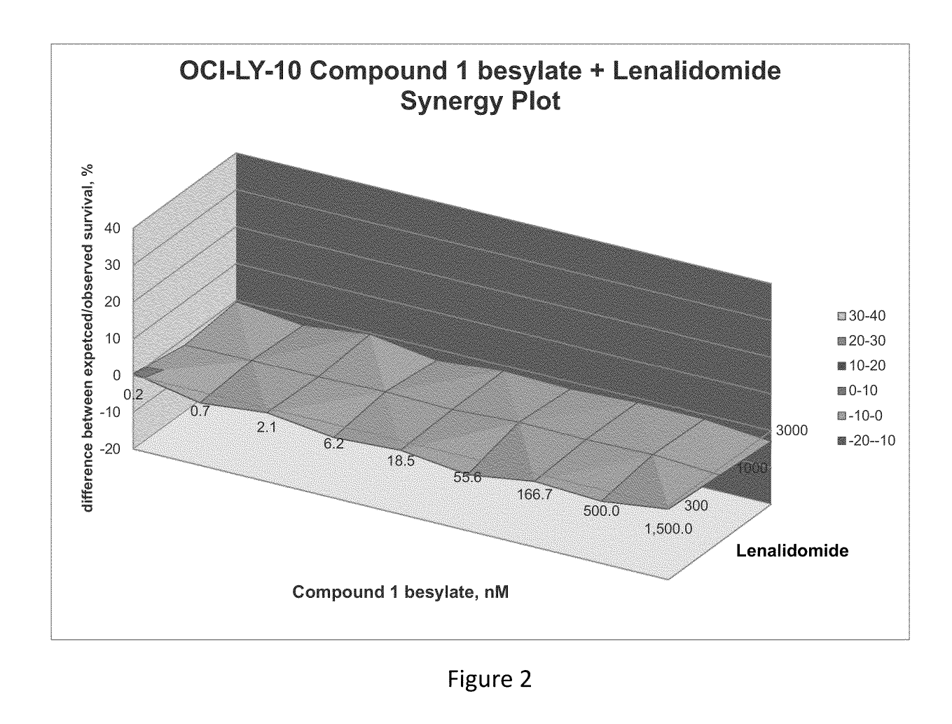

FIG. 1 presents a dose response curve that was observed with Compound 1 besylate and one particular concentration of lenalidomide (3000 nM) in the OCI-LY-10 cell line. The results presented in FIG. 1 appear to show an additive effect, and were confirmed by an additional experiment (data not shown). FIG. 1 therefore can be considered to be representative of data obtained for the combination of Compound 1 besylate and lenalidomide in the OCI-LY-10 cell line. FIG. 2 presents a “volcano” plot of Compound 1 besylate and lenalidomide in the OCI-LY-10 cell line. The volcano plot shown in FIG. 2 was generated using data from experiments run with different concentrations of lenalidomide and is representative of additional experiments in the OCI-LY-10 cell line. The results were confirmed by an additional experiment (data not shown). Compound 1 besylate and lenalidomide thus appear to be additive in the OCI-LY-10 cell line.

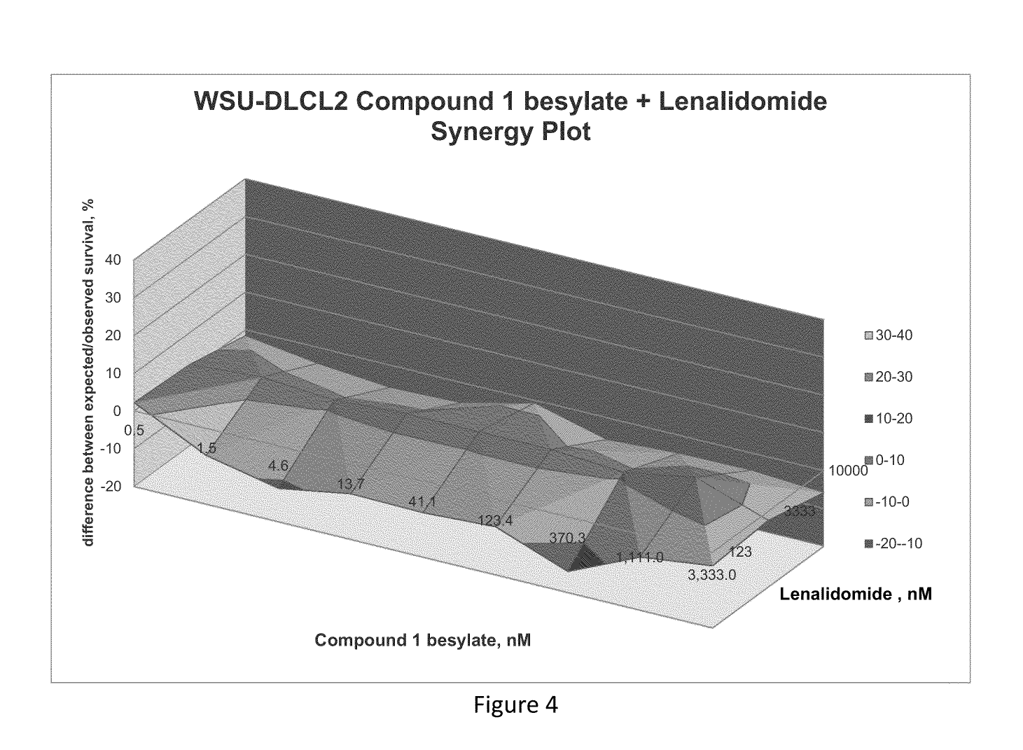

FIG. 3 depicts a particular dose response curve for Compound 1 besylate and one particular concentration of lenalidomide (3333 nM) in the WSU-DLCL2 cell line. The results depicted in FIG. 3 were confirmed by an additional experiment (data not shown). FIG. 3 can thus be considered to be representative of the data obtained for the combination of Compound 1 besylate and lenalidomide in the WSU-DLCL2 cell line. The results presented in FIG. 3 appear to show an additive effect. FIG. 4 presents a “volcano” plot of Compound 1 besylate and lenalidomide in the WSU-DLCL2 cell line. Lenalidomide was also tested in combination with Compound 1 besylate at 37 nM, 111 nM, 370 nM and 1111 nM. No synergy was apparent in this experiment at these concentrations (data not shown). Compound 1 besylate and lenalidomide thus appear to be additive in the WSU-DLCL2 cell line.

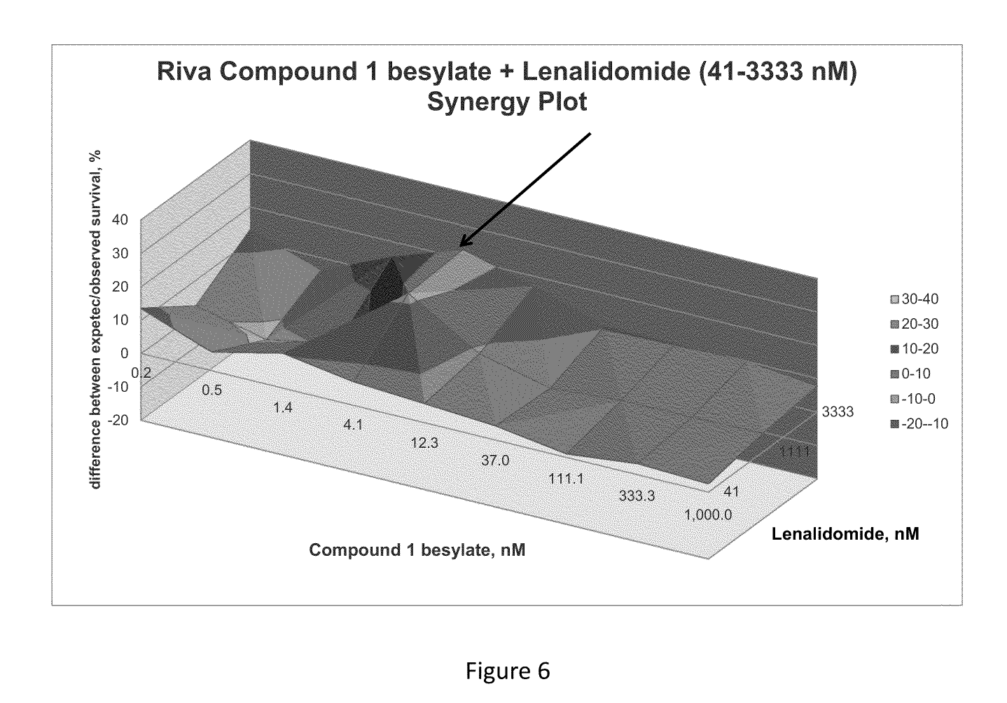

FIG. 5 depicts a particular dose response curve for Compound 1 besylate and lenalidomide (3333 nM) in the Riva cell line. At this concentration, Compound 1 besylate and lenalidomide appear synergistic (indicated by the arrow). FIG. 6 presents a “volcano” plot of Compound 1 besylate and lenalidomide in the Riva cell line at lenalidomide concentrations of 41 nM, 1111 nM and 3333 nM. At certain concentrations, Compound 1 besylate and lenalidomide appear synergistic (indicated by the arrow).

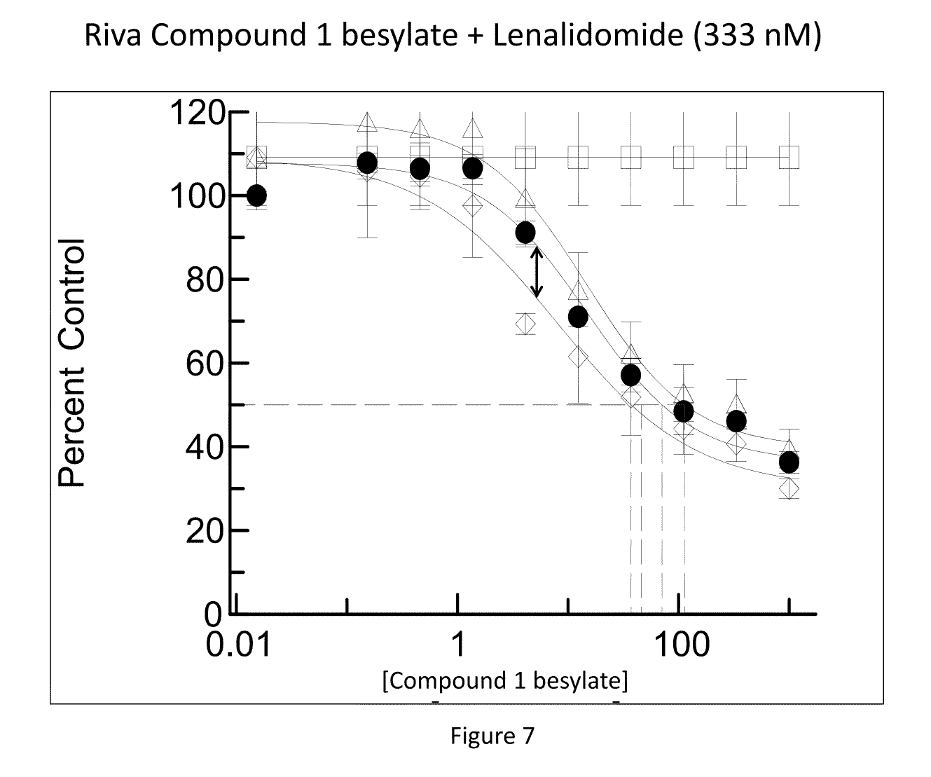

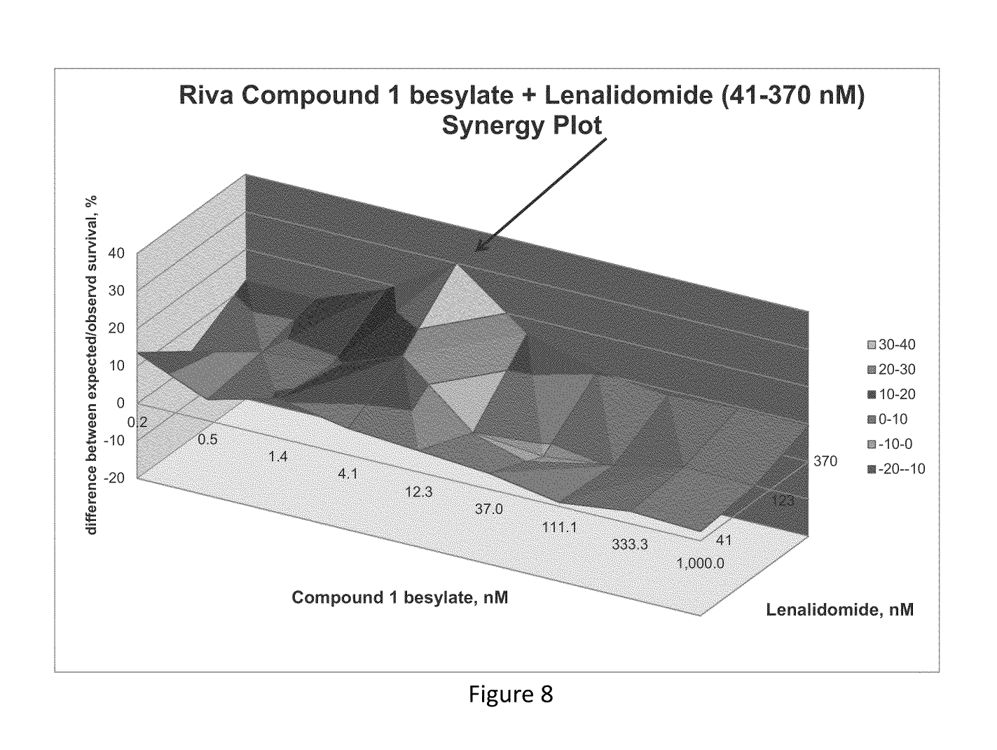

FIG. 7 presents a particular dose response curve for Compound 1 besylate and lenalidomide (333 nM) in the Riva cell line. Compound 1 besylate and lenalidomide in this Figure appear to be synergistic (indicated by the arrow). FIG. 8 presents a “volcano” plot of a particular experiment with Compound 1 besylate and lenalidomide in the Riva cell line at lenalidomide concentrations of 41 nM, 123 nM and 370 nM. Compound 1 besylate and lenalidomide in this Figure appear to be synergistic (indicated by the arrow).

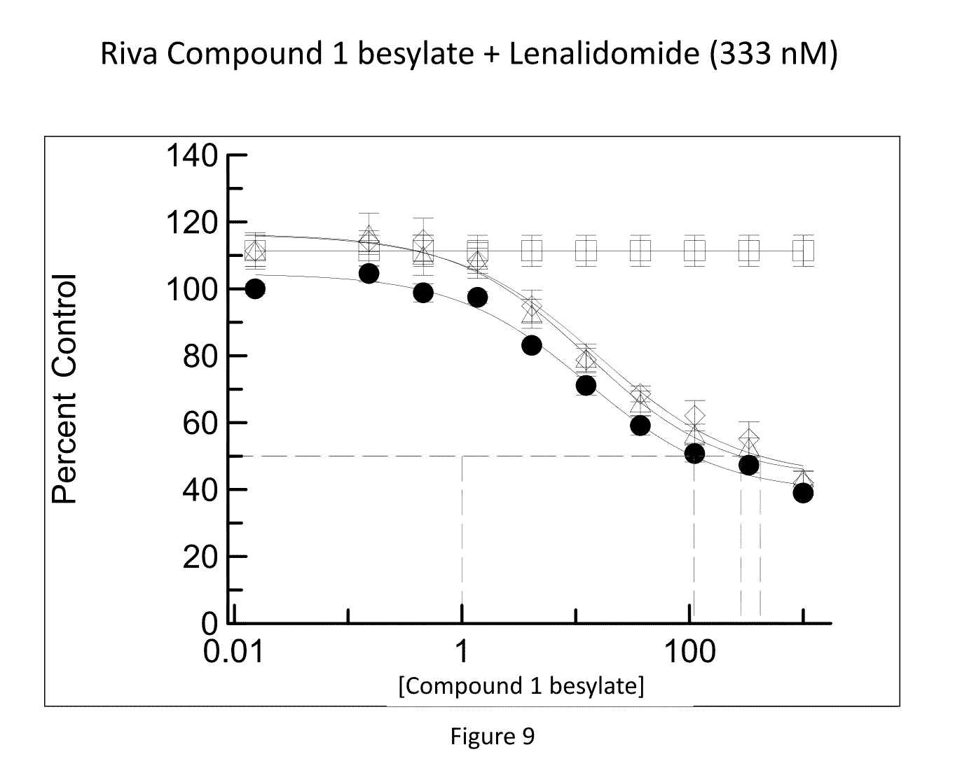

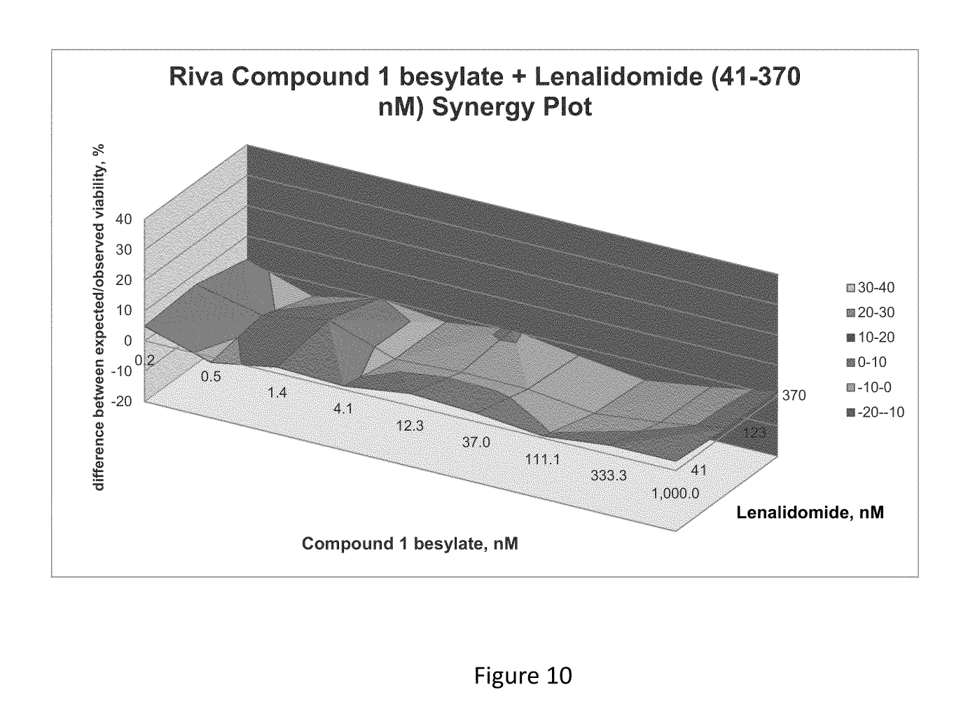

FIG. 9 presents another dose response curve for Compound 1 besylate and lenalidomide (333 nM) in the Riva cell line. Compound 1 besylate and lenalidomide in this Figure appear to be additive. FIG. 10 presents a “volcano” plot of another experiment of Compound 1 besylate and lenalidomide in the Riva cell line at lenalidomide concentrations of 41 nM, 123 nM and 370 nM. Compound 1 besylate and lenalidomide appear additive in FIG. 10. The data in FIGS. 7-10 appear to show that the combination of Compound 1 besylate and lenalidomide is not antagonistic, and may be synergistic.

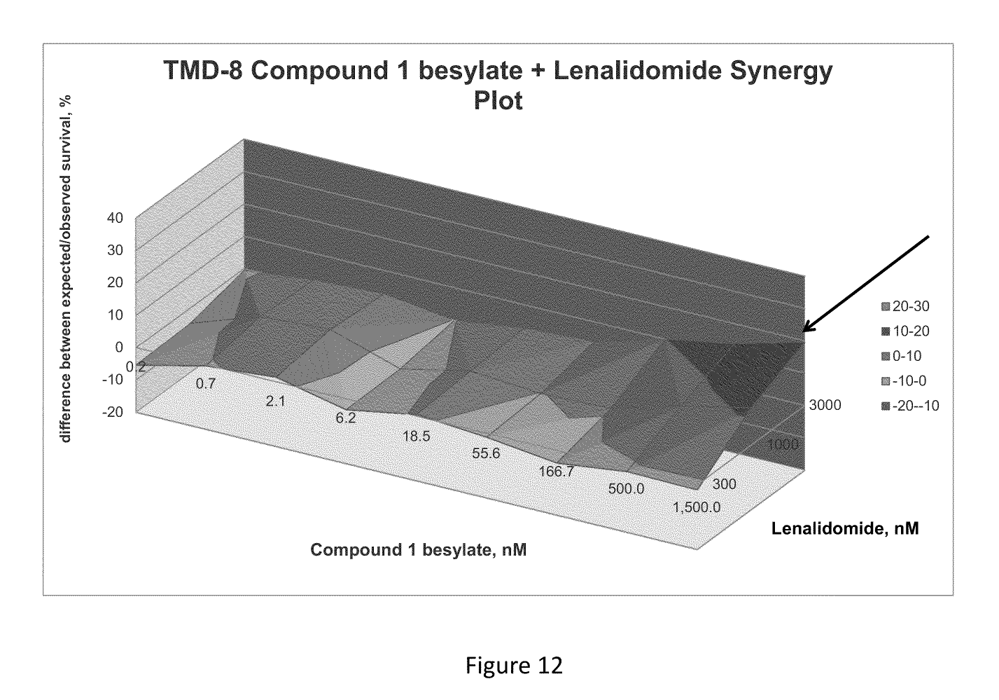

FIG. 11 presents a particular dose response curve for Compound 1 besylate and lenalidomide (3000 nM) in the TMD-8 cell line. Compound 1 besylate and lenalidomide appear synergistic (indicated by the arrow). FIG. 12 presents a “volcano” plot for a particular experiment with Compound 1 besylate and lenalidomide in the TMD-8 cell line at lenalidomide concentrations of 300 nM, 1000 nM and 3000 nM. Compound 1 besylate and lenalidomide in this Figure appear to be synergistic (indicated by the arrow). Lenalidomide was also tested in combination with Compound 1 besylate at concentrations of 100 nM, 300 nM and 1000 nM, and apparent synergy was confirmed (data not shown).

Compound 1 besylate and lenalidomide appear additive in the OCI-LY-10 (FIG. 1 and FIG. 2) and WSU-DLCL2 (FIG. 3 and FIG. 4) cell lines.

Compound 1 besylate and lenalidomide appear synergistic in the Riva cell line at a concentration of 3333 nM of lenalidomide in FIG. 5 and FIG. 6. Compound 1 besylate and lenalidomide in FIGS. 5 and 6 appear to be synergistic (indicated by the arrow). Compound 1 besylate and lenalidomide show some apparent synergy at a concentration of 333 nM of lenalidomide in FIGS. 7 and 8. Compound 1 besylate and lenalidomide appear to be additive at a concentration of 333 nM of lenalidomide in FIGS. 9 and 10. Compound 1 besylate and lenalidomide are confirmed to be synergistic in the TMD-8 cell line in FIG. 11 and FIG. 12. Accordingly, Compound 1 besylate and lenalidomide appear to show synergistic effects in at least the TMD-8 cell line. Some evidence of synergy appears also to be observed in the Riva cell line.

### Example 3

One particular irreversible BTK inhibitor, Compound 2, was screened against 342 kinases to ascertain kinase activity and/or selectivity:

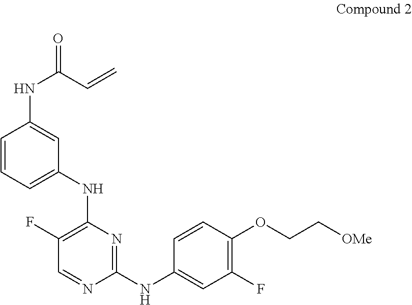 [SMILES: C=CC(=O)Nc1cccc(Nc2nc(Nc3ccc(OCCOC)c(F)c3)ncc2F)c1]

The binding assay system for profiling kinase activity were based upon HotSpot technology (Reaction Biology Corp.; Malvern, Pa., USA) and utilized radio-isotope-based P81 filtration. Compound 2 was dissolved in pure DMSO to make a 10 mM stock solution and serial dilutions were performed to a final 3 μM test concentration. Substrates for the various kinases tested against Compound 2 (substrate information available on the Reaction Biology Corp. website) were prepared fresh daily in Reaction Buffer. Any required cofactors were then added to the substrate solution. The identification and selection of the appropriate cofactor for each kinase is within the ability of a person skilled in the art. See, for example, Handbook of Assay Development in Drug Discovery, Ed. Lisa K. Minor, 2006: CRC Press, Boca Raton, Fla.; Gao et al., “A broad activity screen in support of a chemogenomic map for kinase signalling research and drug discovery,” Biochem J. 2013, 451(2): 313-28; and Eglen et al., “Drug discovery and the human kinome: Recent trends,” Pharmacology & Therapeutics 2011, 130(2): 144-156. The kinase was then added to the substrate solution and gently mixed. Compound 2 (5 nL) was then added to the kinase reaction mixture by acoustical droplet ejection and preincubated for 30 min at room temperature. ³³P-ATP (100 μM) was delivered into the reaction mixture to initiate the reaction. This was followed by incubation at room temperature for 2 h. The reaction was terminated and any unreacted phosphate was washed away using 0.1% phosphoric acid prior to detection utilizing a proprietary technology. The study was performed in duplicate and staurosporine, a non-selective, ATP-competitive kinase inhibitor, was used as the positive control in a 10-dose IC50 mode with 3-fold serial dilutions starting at 1 μM, 50 μM or 100 μM. DMSO was used as the negative control.

Determination of Percent Inhibition.

Percent inhibition of a kinase by a test compound, e.g., Compound 2, was determined according to the following formula: percent inhibition=[(kinase activity of negative control)−(kinase activity in presence of a test compound, e.g., Compound 2)/(kinase activity of negative control)]×100. Percent inhibition was expressed as an average where the assay was performed more than once, e.g., in duplicate.

Table 7 sets forth the average percent inhibition for Compound 2 against various kinases:

TABLE 7
Average Percent Inhibition of Kinases by Compound 2

| | % Enzyme Activity | | |
|---|---|---|---|
| | (relative to DMSO | | |
| | controls) | | Average % |
| Kinase | Run 1 | Run 2 | Inhibition |
| ABL1 | 45.37 | 45.23 | 54.70 |
| ABL2/ARG | 37.26 | 37.07 | 62.84 |
| ACK1 | 22.59 | 22.50 | 77.46 |
| AKT1 | 95.13 | 93.13 | 5.87 |
| AKT2 | 93.65 | 97.89 | 4.23 |
| AKT3 | 110.81 | 102.89 | −6.85 |
| ALK | 82.52 | 83.61 | 16.94 |
| ALK1/ACVRL1 | 124.78 | 123.91 | −24.35 |
| ALK2/ACVR1 | 385.44 | 385.47 | −285.46 |
| ALK3/BMPR1A | 104.30 | 104.51 | −4.41 |
| ALK4/ACVR1B | 140.52 | 143.80 | −42.16 |
| ALK5/TGFBR1 | 106.06 | 110.00 | −8.03 |
| ALK6/BMPR1B | 176.94 | 183.87 | −80.41 |
| ARAF | 102.92 | 100.02 | −1.47 |
| ARK5/NUAK1 | 13.26 | 13.31 | 86.72 |
| ASK1/MAP3K5 | 100.91 | 102.32 | −1.62 |
| Aurora A | 12.26 | 11.03 | 88.35 |
| Aurora B | 13.18 | 12.94 | 86.94 |
| Aurora C | 26.24 | 29.79 | 71.98 |
| AXL | 48.58 | 48.79 | 51.31 |
| BLK | 6.30 | 6.37 | 93.66 |
| BMPR2 | 83.77 | 83.85 | 16.19 |
| BMX/ETK | 0.93 | 1.23 | 98.92 |
| BRAF | 100.83 | 99.10 | 0.04 |
| BRK | 25.10 | 24.97 | 74.96 |
| BRSK1 | 87.10 | 89.40 | 11.75 |
| BRSK2 | 90.99 | 91.69 | 8.66 |
| BTK | 3.69 | 5.40 | 95.45 |
| CAMK1a | 109.10 | 111.65 | −10.38 |
| CAMK1b | 103.91 | 104.95 | −4.43 |
| CAMK1d | 103.60 | 103.13 | −3.36 |
| CAMK1g | 99.95 | 101.49 | −0.72 |
| CAMK2a | 109.14 | 110.06 | −9.60 |
| CAMK2b | 96.40 | 95.81 | 3.89 |
| CAMK2d | 123.75 | 122.81 | −23.28 |
| CAMK2g | 103.51 | 110.85 | −7.18 |
| CAMK4 | 102.16 | 102.35 | −2.26 |
| CAMKK1 | 93.48 | 87.89 | 9.31 |
| CAMKK2 | 74.65 | 71.82 | 26.77 |
| CDC7/DBF4 | 100.26 | 103.78 | −2.02 |
| CDK1/cyclin A | 111.93 | 124.07 | −18.00 |
| CDK1/cyclin B | 87.16 | 87.03 | 12.91 |
| CDK1/cyclin E | 100.38 | 102.23 | −1.30 |
| CDK16/cyclin Y | 104.10 | 103.36 | −3.73 |
| CDK2/cyclin A | 85.83 | 86.86 | 13.66 |
| CDK2/cyclin A1 | 78.25 | 77.74 | 22.00 |
| CDK2/cyclin E | 87.92 | 92.03 | 10.02 |
| CDK3/cyclin E | 78.15 | 76.95 | 22.45 |
| CDK4/cyclin D1 | 95.58 | 96.28 | 4.07 |
| CDK4/cyclin D3 | 95.54 | 94.15 | 5.15 |
| CDK5/p25 | 89.55 | 92.26 | 9.10 |
| CDK5/p35 | 94.97 | 96.06 | 4.49 |
| CDK6/cyclin D1 | 101.41 | 100.42 | −0.92 |
| CDK6/cyclin D3 | 99.55 | 100.13 | 0.16 |
| CDK7/cyclin H | 98.10 | 97.18 | 2.36 |
| CDK9/cyclin K | 81.70 | 82.16 | 18.07 |
| CDK9/cyclin T1 | 87.76 | 91.54 | 10.35 |
| CHK1 | 93.14 | 94.56 | 6.15 |
| CHK2 | 25.85 | 25.28 | 74.43 |
| CK1a1 | 107.76 | 105.57 | −6.67 |
| CK1d | 99.87 | 100.20 | −0.03 |
| CK1epsilon | 101.51 | 102.41 | −1.96 |
| CK1g1 | 88.77 | 90.27 | 10.48 |
| CK1g2 | 89.13 | 85.74 | 12.57 |
| CK1g3 | 84.15 | 85.84 | 15.01 |
| CK2a | 117.05 | 123.24 | −20.15 |
| CK2a2 | 98.25 | 105.38 | −1.81 |
| c-Kit | 77.47 | 79.45 | 21.54 |
| CLK1 | 74.39 | 77.44 | 24.08 |
| CLK2 | 50.21 | 50.65 | 49.57 |
| CLK3 | 90.36 | 95.83 | 6.90 |
| CLK4 | 57.40 | 53.92 | 44.34 |
| c-MER | 66.48 | 66.40 | 33.56 |
| c-MET | 102.62 | 100.36 | −1.49 |
| COT1/MAP3K8 | 101.72 | 100.94 | −1.33 |
| CSK | 81.71 | 81.81 | 18.24 |
| c-Src | 28.17 | 27.88 | 71.97 |
| CTK/MATK | 102.20 | 103.87 | −3.03 |
| DAPK1 | 102.79 | 93.48 | 1.86 |
| DAPK2 | 108.05 | 111.72 | −9.89 |
| DCAMKL1 | 98.43 | 97.52 | 2.02 |
| DCAMKL2 | 100.15 | 99.50 | 0.18 |
| DDR1 | 25.70 | 24.12 | 75.09 |
| DDR2 | 102.90 | 104.85 | −3.87 |
| DLK/MAP3K12 | 74.17 | 80.18 | 22.82 |
| DMPK | 104.46 | 102.05 | −3.25 |
| DMPK2 | 97.36 | 99.56 | 1.54 |
| DRAK1/STK17A | 82.93 | 80.57 | 18.25 |
| DYRK1/DYRK1A | 87.66 | 88.41 | 11.96 |
| DYRK1B | 78.60 | 80.92 | 20.24 |
| DYRK2 | 60.98 | 62.12 | 38.45 |
| DYRK3 | 85.99 | 85.89 | 14.06 |
| DYRK4 | 105.18 | 105.53 | −5.35 |
| EGFR | 19.23 | 20.17 | 80.30 |
| EPHA1 | 99.47 | 101.01 | −0.24 |
| EPHA2 | 84.23 | 84.12 | 15.82 |
| EPHA3 | 96.21 | 100.18 | 1.81 |
| EPHA4 | 92.86 | 88.96 | 9.09 |
| EPHA5 | 89.11 | 93.57 | 8.66 |
| EPHA6 | 95.52 | 102.01 | 1.24 |
| EPHA7 | 60.01 | 64.14 | 37.93 |
| EPHA8 | 94.25 | 93.79 | 5.98 |
| EPHB1 | 79.45 | 79.66 | 20.44 |
| EPHB2 | 104.27 | 106.64 | −5.45 |
| EPHB3 | 99.17 | 98.21 | 1.31 |
| EPHB4 | 81.25 | 81.45 | 18.65 |
| ERBB2/HER2 | 39.81 | 37.50 | 61.34 |
| ERBB4/HER4 | 9.15 | 8.32 | 91.27 |
| ERK1 | 97.44 | 99.93 | 1.32 |
| ERK2/MAPK1 | 105.72 | 102.79 | −4.25 |
| ERK5/MAPK7 | 100.83 | 99.91 | −0.37 |
| ERK7/MAPK15 | 63.77 | 66.05 | 35.09 |
| FAK/PTK2 | 62.84 | 62.86 | 37.15 |
| FER | 88.07 | 88.26 | 11.83 |
| FES/FPS | 63.95 | 65.95 | 35.05 |
| FGFR1 | 41.38 | 39.31 | 59.66 |
| FGFR2 | 37.28 | 35.97 | 63.37 |
| FGFR3 | 32.14 | 31.78 | 68.04 |
| FGFR4 | 66.32 | 63.44 | 35.12 |
| FGR | 39.73 | 40.47 | 59.90 |
| FLT1/VEGFR1 | 83.62 | 79.20 | 18.59 |
| FLT3 | 3.19 | 3.25 | 96.78 |
| FLT4/VEGFR3 | 43.82 | 44.24 | 55.97 |
| FMS | 64.50 | 67.34 | 34.08 |
| FRK/PTK5 | 100.54 | 97.96 | 0.75 |
| FYN | 81.08 | 81.84 | 18.54 |
| GCK/MAP4K2 | 100.27 | 100.02 | −0.14 |
| GLK/MAP4K3 | 102.03 | 108.13 | −5.08 |
| GRK1 | 103.55 | 103.40 | −3.47 |
| GRK2 | 104.15 | 103.91 | −4.03 |
| GRK3 | 99.45 | 100.95 | −0.20 |
| GRK4 | 107.15 | 106.07 | −6.61 |
| GRK5 | 103.03 | 102.15 | −2.59 |
| GRK6 | 102.34 | 103.73 | −3.03 |
| GRK7 | 89.14 | 90.93 | 9.96 |
| GSK3a | 75.97 | 74.98 | 24.53 |
| GSK3b | 121.82 | 122.56 | −22.19 |
| Haspin | 90.56 | 89.91 | 9.77 |
| HCK | 71.99 | 68.97 | 29.52 |
| HGK/MAP4K4 | 98.87 | 98.60 | 1.27 |
| HIPK1 | 87.63 | 91.73 | 10.32 |
| HIPK2 | 95.75 | 98.61 | 2.82 |
| HIPK3 | 112.11 | 118.15 | −15.13 |
| HIPK4 | 93.61 | 93.78 | 6.31 |
| HPK1/MAP4K1 | 85.77 | 88.42 | 12.90 |
| IGF1R | 82.83 | 85.99 | 15.59 |
| IKKa/CHUK | 91.43 | 91.50 | 8.54 |
| IKKb/IKBKB | 95.44 | 97.06 | 3.75 |
| IKKe/IKBKE | 78.26 | 78.18 | 21.78 |
| IR | 84.52 | 84.68 | 15.40 |
| IRAK1 | 76.49 | 74.41 | 24.55 |
| IRAK4 | 88.61 | 86.52 | 12.43 |
| IRR/INSRR | 88.90 | 90.97 | 10.07 |
| ITK | 7.97 | 7.95 | 92.04 |
| JAK1 | 59.70 | 59.03 | 40.63 |
| JAK2 | 108.64 | 114.63 | −11.63 |
| JAK3 | 2.53 | 2.73 | 97.37 |
| JNK1 | 88.14 | 87.66 | 12.10 |
| JNK2 | 92.48 | 95.08 | 6.22 |
| JNK3 | 110.08 | 115.56 | −12.82 |
| KDR/VEGFR2 | 83.35 | 81.24 | 17.70 |
| KHS/MAP4K5 | 94.89 | 90.82 | 7.15 |
| LATS1 | 89.52 | 89.80 | 10.34 |
| LATS2 | 88.40 | 91.16 | 10.22 |
| LCK | 64.67 | 63.36 | 35.99 |
| LCK2/ICK | 100.94 | 95.10 | 1.98 |
| LIMK1 | 60.98 | 61.36 | 38.83 |
| LIMK2 | 101.00 | 100.73 | −0.86 |
| LKB1 | 99.24 | 95.94 | 2.41 |
| LOK/STK10 | 43.10 | 43.01 | 56.94 |
| LRRK2 | 33.02 | 35.12 | 65.93 |
| LYN | 71.67 | 73.24 | 27.54 |
| LYN B | 92.90 | 95.69 | 5.70 |
| MAPKAPK2 | 109.81 | 105.91 | −7.86 |
| MAPKAPK3 | 102.28 | 102.63 | −2.45 |
| MAPKAPK5/PRAK | 109.84 | 113.33 | −11.58 |
| MARK1 | 90.93 | 97.35 | 5.86 |
| MARK2/PAR-1Ba | 99.58 | 97.83 | 1.30 |
| MARK3 | 101.48 | 100.37 | −0.92 |
| MARK4 | 82.17 | 80.87 | 18.48 |
| MEK1 | 109.05 | 111.60 | −10.33 |
| MEK2 | 106.36 | 104.95 | −5.66 |
| MEK3 | 117.30 | 113.34 | −15.32 |
| MEKK1 | 112.92 | 116.55 | −14.74 |
| MEKK2 | 108.13 | 113.65 | −10.89 |
| MEKK3 | 101.68 | 106.39 | −4.03 |
| MELK | 108.80 | 107.34 | −8.07 |
| MINK/MINK1 | 100.72 | 97.49 | 0.90 |
| MKK4 | 116.84 | 116.35 | −16.60 |
| MKK6 | 96.36 | 97.41 | 3.11 |
| MLCK/MYLK | 95.57 | 95.24 | 4.59 |
| MLCK2/MYLK2 | 71.62 | 68.13 | 30.13 |
| MLK1/MAP3K9 | 14.50 | 14.05 | 85.72 |
| MLK2/MAP3K10 | 45.39 | 45.33 | 54.64 |
| MLK3/MAP3K11 | 27.89 | 25.23 | 73.44 |
| MNK1 | 97.78 | 94.26 | 3.98 |
| MNK2 | 83.04 | 83.21 | 16.88 |
| MRCKa/CDC42BPA | 113.51 | 115.15 | −14.33 |
| MRCKb/CDC42BPB | 106.78 | 105.47 | −6.12 |
| MSK1/RPS6KA5 | 94.87 | 99.72 | 2.70 |
| MSK2/RPS6KA4 | 103.35 | 97.38 | −0.36 |
| MSSK1/STK23 | 108.42 | 104.81 | −6.62 |
| MST1/STK4 | 70.20 | 68.65 | 30.58 |
| MST2/STK3 | 91.08 | 88.64 | 10.14 |
| MST3/STK24 | 86.56 | 84.46 | 14.49 |
| MST4 | 97.85 | 104.33 | −1.09 |
| MUSK | 70.16 | 68.05 | 30.89 |
| MYLK3 | 113.48 | 116.49 | −14.98 |
| MYO3b | 103.52 | 101.67 | −2.59 |
| NEK1 | 52.74 | 53.95 | 46.65 |
| NEK11 | 92.53 | 92.84 | 7.32 |
| NEK2 | 102.22 | 106.65 | −4.44 |
| NEK3 | 76.65 | 75.11 | 24.12 |
| NEK4 | 84.64 | 88.33 | 13.52 |
| NEK5 | 60.25 | 60.87 | 39.44 |
| NEK6 | 105.95 | 105.43 | −5.69 |
| NEK7 | 98.29 | 100.34 | 0.68 |
| NEK9 | 83.83 | 84.30 | 15.94 |
| NLK | 99.61 | 102.97 | −1.29 |
| OSR1/OXSR1 | 116.94 | 121.56 | −19.25 |
| P38a/MAPK14 | 105.80 | 107.56 | −6.68 |
| P38b/MAPK11 | 101.64 | 101.17 | −1.41 |
| P38d/MAPK13 | 98.94 | 99.78 | 0.64 |
| P38g | 105.35 | 105.71 | −5.53 |
| P70S6K/RPS6KB1 | 85.89 | 81.02 | 16.54 |
| P70S6Kb/RPS6KB2 | 95.60 | 96.10 | 4.15 |
| PAK1 | 96.27 | 98.58 | 2.58 |
| PAK2 | 95.53 | 94.87 | 4.80 |
| PAK3 | 91.86 | 95.22 | 6.46 |
| PAK4 | 98.70 | 96.40 | 2.45 |
| PAK5 | 122.54 | 132.33 | −27.43 |
| PAK6 | 79.30 | 84.76 | 17.97 |
| PASK | 94.65 | 93.08 | 6.14 |
| PBK/TOPK | 98.58 | 95.27 | 3.07 |
| PDGFRa | 63.93 | 62.78 | 36.65 |
| PDGFRb | 47.74 | 47.41 | 52.42 |
| PDK1/PDPK1 | 106.60 | 105.33 | −5.96 |
| PHKg1 | 85.80 | 85.18 | 14.51 |
| PHKg2 | 117.59 | 111.88 | −14.73 |
| PIM1 | 103.69 | 103.94 | −3.82 |
| PIM2 | 135.59 | 129.41 | −32.50 |
| PIM3 | 103.69 | 99.97 | −1.83 |
| PKA | 85.81 | 85.13 | 14.53 |
| PKAcb | 49.70 | 51.07 | 49.62 |
| PKAcg | 127.92 | 127.73 | −27.82 |
| PKCa | 88.40 | 88.80 | 11.40 |
| PKCb1 | 72.71 | 72.06 | 27.62 |
| PKCb2 | 50.13 | 48.85 | 50.51 |
| PKCd | 100.41 | 96.02 | 1.78 |
| PKCepsilon | 93.22 | 94.21 | 6.29 |
| PKCeta | 108.82 | 116.79 | −12.81 |
| PKCg | 83.88 | 84.16 | 15.98 |
| PKCiota | 105.24 | 105.70 | −5.47 |
| PKCmu/PRKD1 | 74.85 | 75.85 | 24.65 |
| PKCnu/PRKD3 | 80.06 | 79.79 | 20.07 |
| PKCtheta | 83.65 | 84.12 | 16.12 |
| PKCzeta | 99.81 | 95.15 | 2.52 |
| PKD2/PRKD2 | 86.37 | 86.22 | 13.70 |
| PKG1a | 87.99 | 94.55 | 8.73 |
| PKG1b | 85.36 | 87.08 | 13.78 |
| PKG2/PRKG2 | 87.22 | 84.45 | 14.16 |
| PKN1/PRK1 | 93.83 | 92.30 | 6.94 |
| PKN2/PRK2 | 93.30 | 91.42 | 7.64 |
| PKN3/PRK3 | 106.24 | 108.40 | −7.32 |
| PLK1 | 91.11 | 92.77 | 8.06 |
| PLK2 | 86.63 | 86.74 | 13.32 |
| PLK3 | 96.95 | 100.49 | 1.28 |
| PLK4/SAK | 54.75 | 55.16 | 45.04 |
| PRKX | 97.87 | 98.53 | 1.80 |
| PYK2 | 70.59 | 70.13 | 29.64 |
| RAF1 | 83.34 | 83.62 | 16.52 |
| RET | 13.17 | 13.63 | 86.60 |
| RIPK2 | 77.59 | 75.01 | 23.70 |
| RIPK3 | 116.80 | 120.69 | −18.75 |
| RIPK5 | 96.13 | 99.22 | 2.32 |
| ROCK1 | 107.64 | 105.14 | −6.39 |
| ROCK2 | 102.29 | 101.47 | −1.88 |
| RON/MST1R | 103.11 | 101.37 | −2.24 |
| ROS/ROS1 | 13.52 | 13.26 | 86.61 |
| RSK1 | 73.29 | 72.45 | 27.13 |
| RSK2 | 82.57 | 84.26 | 16.59 |
| RSK3 | 85.80 | 85.16 | 14.52 |
| RSK4 | 77.21 | 77.21 | 22.79 |
| SGK1 | 99.71 | 99.55 | 0.37 |
| SGK2 | 71.38 | 76.59 | 26.02 |
| SGK3/SGKL | 99.54 | 105.31 | −2.42 |
| SIK1 | 48.40 | 48.72 | 51.44 |
| SIK2 | 56.26 | 57.07 | 43.34 |
| SIK3 | 91.33 | 92.80 | 7.93 |
| SLK/STK2 | 74.89 | 75.27 | 24.92 |
| SNARK/NUAK2 | 83.70 | 84.92 | 15.69 |
| SRMS | 123.69 | 122.52 | −23.11 |
| SRPK1 | 98.81 | 96.73 | 2.23 |
| SRPK2 | 90.92 | 89.61 | 9.73 |
| SSTK/TSSK6 | 107.37 | 99.84 | −3.60 |
| STK16 | 22.93 | 21.04 | 78.01 |
| STK22D/TSSK1 | 88.04 | 89.72 | 11.12 |
| STK25/YSK1 | 94.47 | 94.10 | 5.72 |
| STK32B/YANK2 | 95.81 | 93.60 | 5.30 |
| STK32C/YANK3 | 104.94 | 107.08 | −6.01 |
| STK33 | 51.19 | 52.54 | 48.14 |
| STK38/NDR1 | 92.71 | 93.70 | 6.79 |
| STK38L/NDR2 | 106.68 | 95.97 | −1.33 |
| STK39/STLK3 | 91.63 | 92.89 | 7.74 |
| SYK | 78.04 | 77.10 | 22.43 |
| TAK1 | 73.39 | 71.98 | 27.32 |
| TAOK1 | 100.50 | 96.25 | 1.63 |
| TAOK2/TAO1 | 98.49 | 94.44 | 3.53 |
| TAOK3/JIK | 94.52 | 90.00 | 7.74 |
| TBK1 | 58.13 | 59.19 | 41.34 |
| TEC | 10.36 | 11.47 | 89.08 |
| TESK1 | 97.22 | 98.52 | 2.13 |
| TGFBR2 | 98.07 | 102.31 | −0.19 |
| TIE2/TEK | 102.88 | 107.55 | −5.21 |
| TLK1 | 102.43 | 103.40 | −2.91 |
| TLK2 | 107.74 | 104.89 | −6.32 |
| TNIK | 66.84 | 67.77 | 32.69 |
| TNK1 | 15.91 | 16.61 | 83.74 |
| TRKA | 117.40 | 117.94 | −17.67 |
| TRKB | 86.53 | 85.00 | 14.24 |
| TRKC | 35.08 | 32.34 | 66.29 |
| TSSK2 | 97.01 | 96.27 | 3.36 |
| TSSK3/STK22C | 132.55 | 132.59 | −32.57 |
| TTBK1 | 101.11 | 102.19 | −1.65 |
| TTBK2 | 102.05 | 99.67 | −0.86 |
| TXK | 0.42 | 0.03 | 99.77 |
| TYK1/LTK | 77.75 | 75.81 | 23.22 |
| TYK2 | 48.20 | 46.63 | 52.58 |
| TYRO3/SKY | 95.11 | 96.84 | 4.03 |
| ULK1 | 100.12 | 101.06 | −0.59 |
| ULK2 | 102.27 | 109.21 | −5.74 |
| ULK3 | 79.58 | 76.98 | 21.72 |
| VRK1 | 84.28 | 89.86 | 12.93 |
| VRK2 | 94.00 | 95.88 | 5.06 |
| WEE1 | 73.06 | 74.89 | 26.03 |
| WNK1 | 112.15 | 112.04 | −12.10 |
| WNK2 | 92.35 | 95.28 | 6.19 |
| WNK3 | 91.35 | 91.97 | 8.34 |
| YES/YES 1 | 18.72 | 18.27 | 81.50 |
| ZAK/MLTK | 83.71 | 85.00 | 15.65 |
| ZAP70 | 110.35 | 108.07 | −9.21 |
| ZIPK/DAPK3 | 109.39 | 113.13 | −11.26 |

### Example 4

### Dose Escalation Study with Rituximab

Capsules comprising N-(3-(5-fluoro-2-(4-(2-methoxyethoxyl)phenylamino)pyrimidin-4-ylamino)phenyl)acrylamide besylate for use in this study correspond to Table 1 in Example 1.

Lenalidomide capsules correspond to those listed in Tables 2 and 3 in Example 1.

Rituximab is provided to the physician/investigator in 10 mg/mL vials comprising 100 mg/10 mL or 500 mg/50 mL. Prior to administration, rituximab is diluted to a dose of 1 mg/mL, 2 mg/mL, 3 mg/mL or 4 mg/mL with either 5% dextrose in water or 0.9% sodium chloride. Rituximab is thereafter administered as a 1 mg/mL to 4 mg/mL infusion according to the dosages set forth in Table 8, below.

Study Design

Subjects with relapsed or refractory CLL or SLL who have at least one prior treatment regimen will be enrolled in a “3+3” dose escalation and expansion study to determine the Not Tolerated Dose (NTD), the Optimal Biologic Effect dose (OBE) and the Maximum Tolerated Dose (MTD) of Compound 1, lenalidomide and rituximab. Approximately 30-42 patients are expected to be enrolled in the study.

Study treatment will be administered in 28-day cycles at specified dose levels as scheduled until disease progression, unacceptable toxicity, or discontinuation for any other reason. Subjects will continue on the starting dose until the preliminary recommended Phase 2 dose (RP2D) is determined, at which point they can be switched to the preliminary RP2D.

N-(3-(5-fluoro-2-(4-(2-methoxyethoxyl)phenylamino)pyrimidin-4-ylamino)phenyl)acrylamide besylate (Compound 1 besylate), lenalidomide and rituximab will be administered according to the cohorts listed in Table 8:

TABLE 8
Study Dosing Schema for Escalating Dose Portion of Study

| | Compound 1 | | |
|---|---|---|---|
| COHORT | besylate | Lenalidomide | Rituximab |
| 1 | 375 mg BID | 5 mg QD on | 6 doses administered as follows: |
| | on days 1-28 | days 1-28 | 375 mg/m² on day 1 for cycle 1 |
| | | starting with | 500 mg/m² on day 1 for cycles |
| | | cycle 2 | 2-6 |
| 2 | 500 mg BID | 5 mg QD on | |
| | on days 1-28 | days 1-28 | |
| | | starting with | |
| | | cycle 2 | |

Within each cohort, subjects will be treated according to the cohorts in Table 8 and will be assessed for safety, tolerability and DLT, as well as pharmacokinetic (“PK”), pharmacodynamic (“PD”), and disease response.

Compound 1 besylate and lenalidomide are administered PO according to Table 8.

Rituximab is administered as a single intravenous (IV) infusion. The initial infusion during cycle 1 will be administered at 375 mg/m²; subsequent infusions during cycles 2 through 6 will be administered at 500 mg/m². Administration of rituximab will occur on day 1 of each 28-day cycle. Following the cycle 6 infusion, rituximab will be discontinued. Subjects may continue on treatment with Compound 1 besylate and/or lenalidomide if appropriate. The first infusion of rituximab will be at a rate of 50 mg/hr. In the absence of infusion toxicity, the infusion rate will be increased by 50 mg/hr increments every 30 minutes, to a maximum of 400 mg/hr. Each subsequent infusion will be initiated at 100 mg/hr. In the absence of infusion toxicity, the infusion rate will be increased by 100 mg/hr increments at 30 minute intervals to a maximum of 400 mg/hr.

If a tolerated and/or desired response is not achieved after 2 cycles on lenalidomide at 5 mg (i.e., after completion of at least 3 total 28-day cycles), the dose of lenalidomide can be escalated to 10 mg QD.

We claim:

1. A method of treating, stabilizing or lessening the severity or progression of one or more diseases and conditions associated with BTK comprising administering to a patient in need thereof an irreversible BTK inhibitor and lenalidomide, wherein the irreversible BTK inhibitor has not more than about 50% inhibition of a kinase selected from c-Kit, PDGFRa, RIPK2, HCK, EPHA6, LYN, CSK, LCK, ZAK/MLTK, LYN B, FRK/PTK5, FYN, BRAF, RIPK3, ARAF and SRMS, or combinations thereof.

2. The method according to claim 1, wherein the irreversible BTK inhibitor has not more than about 30% inhibition of a kinase selected from c-Kit, RIPK2, HCK, EPHA6, LYN, CSK, ZAK/MLTK, LYN B, FRK/PTK5, FYN, BRAF, RIPK3, ARAF and SRMS, or combinations thereof.

3. The method according to claim 1, wherein the irreversible BTK inhibitor has not more than about 10% inhibition of a kinase selected from EPHA6, LYN B, FRK/PTK5, BRAF, RIPK3, ARAF and SRMS, or combinations thereof.

4. The method according to claim 1, wherein the irreversible BTK inhibitor has a percent inhibition of LYN that is not more than about 20-30%.

5. A method of treating, stabilizing or lessening the severity or progression of one or more diseases and conditions associated with BTK comprising administering to a patient in need thereof Compound 1 (N-(3-(5-fluoro-2-(4-(2-methoxyethoxyl)phenylamino)pyrimidin-4-ylamino)phenyl)acrylamide): 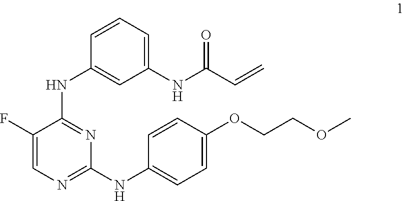 [SMILES: C=CC(=O)Nc1cccc(Nc2nc(Nc3ccc(OCCOC)cc3)ncc2F)c1]
or a pharmaceutically acceptable salt thereof, lenalidomide and an anti-CD20 antibody.

6. The method according to claim 5, wherein the disease or condition associated with BTK is selected from chronic lymphocytic leukemia and small lymphocytic lymphoma.

7. The method according to claim 6, wherein Compound 1 is administered twice a day.

8. The method according to claim 7, wherein Compound 1 is administered on each day of a 28-day cycle.

9. The method according to claim 8, wherein the anti-CD20 antibody is rituximab.

10. The method according to claim 9, wherein rituximab is administered once during a 28-day cycle.

11. The method according to claim 10, wherein rituximab is administered as an intravenous infusion.

12. The method according to claim 11, wherein lenalidomide is administered once a day starting with the second 28-day cycle.

13. The method according to claim 7, wherein Compound 1 is in the form of a benzenesulfonic acid salt.

14. The method of claim 13, wherein each of Compound 1 and lenalidomide is administered as an oral dosage form.

15. A method of preventing, treating, stabilizing or lessening the severity or progression of a disease or disorder selected from the group consisting of chronic lymphocytic leukemia and small lymphocytic lymphoma, the method comprising administering to a patient in need thereof therapeutically effective amounts of each of Compound 1, or a pharmaceutically acceptable salt thereof, lenalidomide and an anti-CD20 antibody, wherein the therapeutically effective amount of Compound 1 is about 750 mg to about 1000 mg per day.

16. The method according to claim 15, wherein the anti-CD20 antibody is rituximab.

17. The method according to claim 15, wherein the therapeutically effective amount of Compound 1 is about 375 mg BID.

18. The method according to claim 15, wherein the therapeutically effective amount Compound 1 is about 500 mg BID.

19. The method according to claim 16, wherein the therapeutically effective amount of rituximab is about 375 mg/m².

20. The method according to claim 16, wherein the therapeutically effective amount of rituximab is about 500 mg/m².

21. The method according to claim 15, wherein lenalidomide is administered starting with the second 28-day cycle.

22. The method according to claim 21, wherein the therapeutically effective amount of lenalidomide is about 5 mg.

23. The method according to claim 21, wherein the therapeutically effective amount of lenalidomide is about 10 mg.

24. A system for treating, stabilizing or lessening the severity of one or more diseases or conditions associated with BTK, the system comprising Compound 1, or a pharmaceutically acceptable salt thereof, lenalidomide and an anti-CD20 antibody.

25. The system according to claim 24, wherein the anti-CD20 antibody is rituximab.
